(19) United States
(12) Patent Application Publication
(10) Pub. No.: US 2003/0105026 A1
(43) Pub. Date: Jun. 5, 2003

(54) Compounds comprising disulfide-containing peptides and nitrogenous bases, and medical uses thereof

(76) Inventors: Leonid A. Kozhemyakin, St. Petersburg (RU); Andrew L. Kozhemyakin, St. Petersburg (RU)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/072,815

(22) Filed: Feb. 7, 2002

Related U.S. Application Data
(63) Continuation-in-part of application No. 09/903,443, filed on Jul. 11, 2001.
Application No. 09/903,443 is a continuation-in-part of application No. 09/887,537, filed on Jun. 22, 2001, now abandoned.

(30) Foreign Application Priority Data
Feb. 8, 2001 (RU) 2001103535/14

(51) Int. Cl. A61K 38/05; A61K 31/522; A61K 31/513; A61K 31/708; A61K 31/7072

(52) U.S. Cl. 514/18; 514/263.31; 514/269; 514/48; 514/51

(57) ABSTRACT

The invention relates to new compositions and medical uses, such as anti-infectious pharmacology. The compositions include salts and compounds of GSSG including at least one counterion comprising a nitrogenous base. Examples of such nitrogenous bases include DNA bases, nucleosides of DNA bases, nucleotides of DNA bases, RNA bases, nucleosides of RNA bases, nucleotides of RNA bases, inosine, nucleotides of inosine, and homologues, analogues and derivatives thereof. The invention is also directed to methods for treatment and prevention of infectious diseases such as viral hepatitis B and C, AIDS and herpes.

### RELATED APPLICATIONS

This application is a continuation-in-part of U.S. patent application Ser. No. 09/903,443, filed Jul. 11, 2001, which is a continuation-in-part of U.S. patent application Ser. No. 09/887,537, filed Jun. 22, 2001. This application also claims priority to Russian application no. 2001103535/14, filed Feb. 8, 2001, the entire contents of which is incorporated herein by reference.

### FIELD OF THE INVENTION

The invention relates to new compositions and medical uses, such as anti-infectious pharmacology. The invention also relates to the development of new therapeutic agents based on active metabolites of peptides, nucleosides and nucleotides that are intended to be used for treatment and prevention of infectious diseases such as viral hepatitis B and C, AIDS and herpes.

### BACKGROUND OF THE INVENTION

Infections caused by hepatitis viruses B (HBV) and C (HCV) are spread at a considerably faster rate than infections caused by HIV. The infection rate by HBV and, especially, HCV are growing with each coming year. In some world regions more than 10% of adult population are infected with hepatitis C. Additionally, HCV has a high potential for progressing to a chronic state and possibly being the cause for the entire range of severe liver diseases. In particular, HCV is responsible for more than 70% of chronic hepatitis cases, 40% of liver cirrhosis and 60% of hepatocellular carcinoma. (J.Hepatol. 1999, 30:956-961.)

Basic mechanisms for the progression to chronic viral hepatitis, particularly, for hepatitis C, include “viral elusion” of immune surveillance as well as an emergence of multiple, simultaneously present, viral variants with an altered but similar genome, known as so-called “quasi-species”. Recent studies show the presence of viral-specific T-lymphocytes in the liver cells, indicating an immune-mediated pathogenesis of hepatitis C. (Naoumov N V., Gastroenterology, 1999, 117: 1012-1014.) Moreover, a dependence of the severity of liver morphology (Knodell's index) on the content of intrahepatic CD4+ cells was found. (Tran A et al., Dig.Dis.Sci. 1997, 42: 2495-2500.) T-cell response is of primary importance, as CD4+, CD8+ and CD16/56+ response on HCV NS3 protein was described as being highly significant for spontaneous hepatitis C recuperation and for a positive outcome of antiviral therapy. (Gerlach J T et al., Gastroenterology, 1999, 117: 933-941; Cramp M E, et al., Gut 1999, 44: 424-429.)

The immune response is known to be diverse depending on dominant participation of the CD4+ clones of T-lymphocytes, i.e. T-helpers 1 (Th1) and T-helpers 2 (Th2) which differ in the cytokines produced and on immune response activation through a cell or humoral pathway. Activation of Th1 producing interferon-gamma (IFN-γ), interleukin-2 (IL-2), tumor necrosis factor alpha and beta (TNF-α and β) results in stimulation of T-lymphocyte and macrophage functions, i.e. cell-mediated immune response, which is crucial for antiviral defense. Activation of Th2 releasing IL-4, IL-5, IL-6, IL-9, IL-10 and IL-13 triggers humoral immunity. Therefore, the altered balance of cytokine production between Th1/Th2 cells is of primary importance for immune pathogenesis of HBV, HCV and HIV. The prevailing participation of Th2 type cytokines is associated with viral persistence and disease progression. Activation of Th1-dependent cytokine production facilitates spontaneous recovery from acute viral infections and/or promotes therapeutic effectiveness of agents for viral elimination and for restoring functional capacities of impaired organs. (Eckels, D. D. et al., Hum. Immunol., 1999, 60: 187-199; Ferrari, C. et al., Cancer Res., 1998, 154: 330-336; Fan, X. G. et al., Mediators Innamm. 1998, 7: 295-297; Rehermann, B. et al. Cur. Top. Microbiol. Immunol. 2000, 242: 299-325.)

The function of pleiotropic cytokine, IL-12 can be a factor for host immune response regulation. By regulating the Th1/Th2 balance, IL-12 modulates macrophage functions, especially for liver resident macrophages (Kuppfer's cells). For chronic hepatitis C or B, IL-12 activates cytokine release in the Th1 cells, inhibiting corresponding Th2 function. Chronic forms and an unfavorable courses of viral hepatitis are accompanied with a decreased IL-12 content. (Schlaak, J. F. et al., J. Med. Virol. 1998, 56: 112-117.) Thus, T-cell immunity relating to the course and outcome of HBV and HCV infections and successful treatment is dependent on endogenous production of appropriate cytokines capable of responding to an antigen stimulus. The host immune response and production of these cytokines, which are in competition with the generation of new “quasi-species”, both can determine the capability for modulating the targeting and intensity of the host immune response.

Antiviral chemotherapeutics for viral hepatitis are related to three major groups: (1) reverse transcriptase inhibitors including nucleoside analogues, nucleotide analogues and non-nucleoside analogues; (2) protease inhibitors; and (3) pyrophosphate analogues. Various studies show that for hepatitis B, the most effective agents in the nucleoside analogue group are Lamivudine (GlaxoSmithKline, Research Triangle Park, N.C.), Azidothymidine (“AZT”, GlaxoSmithKline, Research Triangle Park, N.C.) and Famcyclovir (Novartis Pharmaceuticals, East Hanover, N.J.), and for hepatitis C, the most widely used therapeutics are Ribavirin (Rebetol®, Kenilworth, N.J.) and Azidothymidine (GlaxoSmithKline, Research Triangle Park, N.C.). (Semenenko, T. A., Inf. Bull. No. 1(8), 2000; Bartholomew, M. M. et. al., Lancet, 1997, 349 No. 9044, 20-22; Di Bisceglie, A. M. et. al. Ann. Intern. Med., 1995, 123, 897-903.)

Treatment of chronic hepatitis B with the nucleoside analogue, Lamivudine, appeared promising. (Schiff, E. R., J. Med. Virol., 2000 Jul; 61(3): 386-91; Dienstag, J. L. et. al., N. Engl. J. Med. 1999 Oct 21; 341(17): 1256-63; Yao, G. B. et al., DDW 1999.) Multiple side effects of Lamivudine, however, are well known. Example side effects include nausea, vomiting, diarrhea, hepato- and nephrotoxicity, neutropenia, thrombocytopenia, anemia, and dermatological reactions such as skin rash and alopecia.

Ribavirin applied as a single-agent therapy does not provide a satisfactory therapeutic effect, although a combined administration of high doses of Interferon and Ribavirin for an extended period of time (i.e. not less than 12 months), may be a promising therapy. (Pol S. et. al., Hepatology, 2000 Jun; 31(6): 1338-44.) Ribavirin, however, is not free of serious side effects such as bronchial spasm, pulmonary edema, high blood pressure, anemia, skin rash, and asthenia. The main shortcoming of Ribavirin, however, is its low antiviral activity and absence of hepatoprotective effects which is important for viral hepatitis treatment.

AZT agents were promising drugs for HIV and herpetic infections. (The Medical Management of AIDS, 4^{th} edition. Sande M. et al., Saunders Company, 1995.) The widespread administration of AZT was hindered, however, after rapid development (after 2-3 therapeutic cycles) of severe complications such as hemo- and immunosuppression, allergic dermatosis and mucosa candidiasis.

The unique features of oxidized glutathione (GSSG) has been previously discovered to stimulate endogenous production of cytokines and hemopoietic factors. (Russian Federation Patent No. 2089179, Kozhemyakin, L. A. et al.)

It has been shown that stabilization of the GSSG disulfide bond extends the half-life considerably, of exogenously introduced GSSG oxidized (disulfide) form in biological media than in previously discovered biological and pharmacological applications of GSSG. (Russian Federation Patent No. 2089179, Kozhemyakin, L. A. et al.; Russian Federation Patent No. 2153350, Kozhemyakin, L. A. et al.) In biological media GSSG is metabolized by NADP-H⁺-dependent glutathione-reductase that cleaves the GSSG disulfide bond forming two molecules of reduced glutathione (GSH).

It has been previously disclosed that to prolong the exogenously introduced GSSG half-life in disulfide form in biological media, inosine or inosine-monophosphate (IMEP) was added to protect GSSG against “attacks” of NADP-H⁺-dependent glutathione-reductase. (Russian Federation Patent No. 2153350, Kozhemyakin, L. A. et al.) Russian Federation Patent No. 2153350 also discloses a group of pharmaceutically acceptable substances containing GSSG as an active substance in the form of a composite that is inorganic in nature in that they contain alkaline (Na, Li) or alkaline-earth (Ca, Zn, Mg) metals as cations.

In summary, typical previous treatments for hepatitis B and C involved administration of recombinant interferons and nucleoside analogues. An approved combined therapy program (Interferon, 3 mln IU, 3 times a week for 6 months, +Ribavirin) was considered, until recently, as the “golden standard” treatment, provided the disease was in remission for more than 30% of the patients. Currently, the disease is in remission for only 12-15% of patients. Such negative development of the therapeutic efficacy of conventional antiviral combination is caused (not excluding other mechanisms) by the fact that interferons are high-molecular (large-size) proteins which induce antibody production. Therefore, the higher the dosage and longer such treatment is applied within the human population (taking into account the rate of galloping viral hepatitis epidemic), the lower its efficacy as time progresses and the higher the incidence of complications manifested as immune autoaggression.

### SUMMARY OF THE INVENTION

One aspect of the invention provides low-molecular weight compounds for treating diseases, such as infectious diseases, and related treatment methods. In one embodiment, these compounds can be structural and functional analogues of key cell regulatory factors (active metabolites) possessing simultaneously antiviral, immunomodulating, hepato- and hemoprotective effects to achieve a medically desirable result. Preferably, the compounds of the invention include oxidized glutathione (GSSG), e.g., as an oxidized salt and at least one counterion containing a nitrogenous base.

In another aspect of the invention, the antiviral agents of the 1st group, i.e. Lamivudine, Azidothymidine, Famcyclovir, are chosen as prototypes for an efficacy assessment in comparison with the compositions of the present invention.

In another aspect of the present invention, a therapeutic strategy for viral hepatitis and AIDS includes the provision of immunorehabilitating agents. These agents can simultaneously (1) act as antiviral agents, i.e. regulate endogenous cytokine production (even to reproduce their effects in conditions of helper and cytotoxic activity blocking); and (2) selectively inhibit viral replicative activity.

It is to be understood that although theoretical mechanisms may be proposed as a basis for a therapeutic effect, the inventions disclosed herein are not limited to any particular theory or mechanism.

Another aspect of the invention comprises compositions comprising organic salts of GSSG, obtained through the formation of ion bonds between GSSG and at least one organic counterion. One embodiment provides a composition comprising an oxidized glutathione salt, at least one counterion of the oxidized glutathione comprising a nitrogenous base including one or more of DNA bases, nucleosides of DNA bases, nucleotides of DNA bases, RNA bases, nucleosides of RNA bases, nucleotides of RNA bases, inosine, nucleotides of inosine, and homologues, analogues and derivatives thereof.

In one embodiment, the GSSG salt comprises an anion having a counterion comprising a protonated nitrogen atom of the nitrogenous base. The anion can comprise a carboxylate of a glycine residue of GSSG. In another embodiment, the protonated nitrogen is part of a heterocycle of the nitrogenous base. Exemplary salts include GSSG and inosine-monophosphate (GSSG·IMP); GSSG and uridine-monophosphate (GSSG-UMP).

In one embodiment, the GSSG salt comprises D-forms of GSSG.

In one embodiment, the composite comprises GSSG as a cation having a counterion comprising a nucleotide. In one embodiment, the salt comprises an ionic bond formed. For example, an ionic bond can be formed at pH=5-7 between a protonated amino group (NH₃⁺) of a glutamic acid residue (pH=9.47) of the GSSG cation, and an ionized primary phosphate group of the nucleotide anion (pH=1.0).

Another aspect of the present invention comprises a composition comprising a compound comprising an oxidized glutathione covalently bonded to inosine.

Another aspect of the present invention comprises a composition comprising a compound including a covalent bond between GSSG and nucleotide-monophosphates such as inosine-5¹-phosphate, uridine-5¹-phosphate, cytidine-5¹-phosphate, thymidine-5¹-phosphate, adenosine-5¹-phosphate and guanosine-5¹-phosphate. In another embodiment, the covalent bond comprises a phosphoramide bond formed between an amino group of GSSG and a phosphoric acid group of a nucleotide-monophosphate.

In one embodiment, the composition comprises a covalent bond between salts of D-forms of GSSG and nucleotide-monophosphates (e.g. see FIGS. 22 and 23).

The following terminology is used herein, as accepted in the art.

“Metabolism” is the sum of all biochemical reactions within cells of living organism. (Robert C Bohinski “Modem Concepts in Biochemistry”, 4^{th} edition, 1987.) “Active metabolite”, is intended to mean any biochemical compound produced by metabolism (typically short-chain peptides comprising 2 to 9 amino acids), and their transformations can determine the direction and activity of various metabolic processes.

“Apoptosis” is a form of genetically programmed cell death. (Harrison's Principles of Internal Medicine, 14^{th} edition, p. 511, 1998; Apoptosis: a role in neoplasia, C. D. Gregory, 1996; The Molecular Biology of Apoptosis, D. L. Vaux et al., Proc. Natl. Acad. Sci. USA 93, 1996.) Aged cells can be removed from the body through apoptotic mechanisms. Cell death can be induced during embryogeny as well as death of “waste” activated immune cells. Apoptosis can be a physiologic cell suicide.

Cytokines are intended to mean body regulatory substances, often proteins, and are usually produced by immunocompetent cells. Cytokines are a factor in immune response, hemopoiesis and apoptotic processes. Cytokines include interleukins (IL—e.g. IL-1β, IL-2, IL-4, IL-5, IL-6, IL-9, IL-10, IL-12 and IL-13), interferons (IFN—e.g. IFN-γ), tumor necrosis factors (TNF—e.g. TNF-α and β) and erythropoietins, among others. The rate and content of cytokine production determine the nature of pathogenesis of various diseases including infectious diseases. (Harrison's Principles of Internal Medicine, 14^{th} edition, p. 511, 1998; Cytokines in oncohematology, L. A. Grachyova, Moscow, 1996).

Immunocompetent cells marked as: CD3+; CD4+; CD8+; CD16/56+ (NK-cells), are intended to mean different types (differentia markers) of T-lymphocytes specific for a given cell type, and form the host immune response to an antigen or other pathogenic elements.

Knodell's histologic activity index (HAI) proposed by R. G. Knodell et al. (1981) can indicate the character and intensity of inflammatory and necrotic impairments in liver cells (hepatocytes) as well as the character of morphologic alterations in liver tissue. Knodell's index can also determine the activity rate of liver tissue impairment semi-quantitatively. This index involves a point-based system, for different types of hepatitis, including viral hepatitis B and C. A score of 1-3 points indicates minimal activity; 4-8 indicates low to moderate activity; 9-12 points indicates moderate activity; and 13-18 points indicates severe activity, usually found in cirrhotic liver alterations.

Polymerase chain reaction (PCR) is a technique capable of detecting the presence (+) or absence (−) of replicative activity of DNA and RNA viruses, such as hepatitis B virus (HUBV) or hepatitis C virus (HCV). Quantitative PCR can also be used to estimate the number of virus copies per 1 ml of blood. For example, high replicative activity, i.e. active viral reproduction in the host organism (high viral “load”), would indicate a severe infectious clinical course. As another example, termination of virus replicative activity or its considerable diminution would indicate antiviral activity of the drugs administered for viral diseases.

A non-structural protein of hepatitis C virus (NS3) is a key regulatory viral protein necessary for viral replication, and thus, activity. This protein is a multifunctional enzyme possessing three catalytic features: (1) protease; (2) helicase (“untwisting” the host DNA for virus implantation); and (3) ATP-ase, i.e. capability of ATP cleavage to supply energetically viral replication (e.g. “cap” synthesis). (P. Galinari et al., J.Virol., August 1998, p. 6758-6769, Vol. 72, No.8.)

Other advantages, novel features, and objects of the invention will become apparent from the following detailed description of the invention when considered in conjunction with the accompanying drawings. Some of the accompanying drawings are schematic and are intended to indicate a formalism for the representing a chemical structure, as understood by those of ordinary skill in the art. For purposes of clarity, not every component is labeled in every figure, nor is every component of each embodiment of the invention shown where illustration is not necessary to allow those of ordinary skill in the art to understand the invention.

### BRIEF DESCRIPTION OF THE FIGURES

FIG. 1 is a structural formulation of the organic salt of bis-(γ-L-glutamyl)-L-cysteinyl-bis-glycine (GSSG) with adenine;

FIG. 2 is a structural formulation of the organic salt of GSSG with guanine;

FIG. 3 is a structural formulation of the GSSG organic salt with thymidine;

FIG. 4 is a structural formulation of the GSSG organic salt with uracil;

FIG. 5 is a structural formulation of the GSSG organic salt with cytosine;

FIG. 6 is a structural formulation of the GSSG organic salt with 5-methyl-cytosine;

FIG. 7 is a structural formulation of the GSSG organic salt with 4-thiouracil;

FIG. 8 is a structural formulation of the GSSG organic salt with dihydrouracil;

FIG. 9 is a structural formulation of a compound (organic salt), including ionic species GSSG and inosine (9-β-D-ribofuranosylhypoxanthine);

FIG. 10 is a structural formulation of a compound (organic salt), including ionic species GSSG and thymidine (3-β-D-2-deoxyribofuranosylthymine);

FIG. 11 is a structural formulation of a compound (organic salt), including ionic species GSSG and uridine (3-β-D-ribofuranosyluracil);

FIG. 12 is a structural formulation of a compound (organic salt), including ionic species GSSG and guanosine wherein R is a ribose residue;

FIG. 13 is a structural formulation of a compound (organic salt), including ionic species GSSG and adenosine, wherein R is a ribose residue;

FIG. 14 is a structural formulation of a compound comprising a covalent link between Na₂-GSSG and uridine-5¹-phosphate (UMP);

FIG. 15 is a structural formulation of a compound comprising a covalent link between Na₂-GSSG and cytidine-5¹-phosphate (CMP);

FIG. 16 is a structural formulation of a compound comprising a covalent link between Na₂-GSSG and thymidine-5¹-phosphate(TMP);

FIG. 17 is a structural formulation of a compound comprising a covalent link between Na₂-GSSG and adenosine-5¹-phosphate (AMP);

FIG. 18 is a structural formulation of a compound comprising a covalent link between Na₂-GSSG and guanosine-5¹-phosphate (GMP);

FIG. 19 is a structural formulation of a compound comprising a covalent link between Zn₂-GSSG and thymidine-5¹-phosphate (TMP);

FIG. 20 is a structural formulation of a compound comprising a covalent link between Ag₂-GSSG and uridine-5¹-phosphate (UMP);

FIG. 21 is a structural formulation of a compound comprising a covalent link between Li₂-GSSG and guanosine-5 ¹-phosphate (GMP);

FIG. 22 is a structural formulation of a compound comprising a covalent link between D-form of Na₂-GSSG and cytosine-monophosphate (D-glutamic acid);

FIG. 23 is a structural formulation of a compound comprising a covalent link between D-form of Na₂-GSSG and cytosine-monophosphate (D-cysteine);

FIG. 24 is a structural formulation of a compound (organic salt), including ionic species GSSG D-form and uracil;

FIG. 25 is a structural formulation of a compound (organic salt), including ionic species GSSG D-form and inosine;

FIG. 26 is a structural formulation of a combined organic salt, including ionic species GSSG and nitrogenous bases of purine and pyrimidine origin;

FIG. 27 is a structural formulation of a combined organic salt, including ionic species GSSG and nucleosides of purine and pyrimidine origin;

FIG. 28 is a structural formulation of a combined compound, comprising covalent links among aminogroups of the GSSG salts and phosphamide groups of nucleotides of purine and pyrimidine origin;

FIG. 29 is a structural formulation of disodium (dilithium) salt of 9-β-D-ribofuranosyl-5′-phosphoryl-N-bis-(γ-L-glutamyl)-L-cysteinyl-bis-glycine obtained by formation of a covalent link between Na₂(Li₂)-GSSG and inosine-monophosphate (IMP) (see Example 1);

FIG. 30 shows the development of clinical, laboratory and morphologic indices of K (chronic hepatitis B, Example 7), treated with GSSG·inosine, DNA +/− and RNA +/− means positive or negative PCR for DNA HBV and RNA HCV, respectively;

FIG. 31 shows the development of clinical, laboratory and morphologic indices of Z. (Example 10), treated with GSSG·inosine, DNA +/− and RNA +/− means positive or negative PCR for DNA HBV and RNA HCV, respectively;

FIG. 32 is a thoracic X-ray image (left lung) before treatment (October 1999) (see Example 16);

FIG. 33 is a thoracic X-ray image (left lung) after treatment (January 2000) (see Example 16);

FIG. 34 shows a therapeutic efficacy of studied hepatoprotective agents for experimental dichlorethane hepatitis (Example 17), 1=dichloroethane, 2=dichloroethane+Legalon®, 3=dichloroethane+Essentiale®, 4=dichloroethane+GSSG·inosine;

FIG. 35 shows a therapeutic efficacy of studied hepatoprotective agents for experimental Acetaminophen hepatitis (Example 17), 1=Acetaminophen, 2=Acetaminophen+Legalon®, 3=Acetaminophen+Essentiale®, 4=Acetaminophen+CSSG·inosine;

FIG. 36 shows a therapeutic efficacy of studied hepatoprotective agents for combined (Dichloroethane+Acetaminophen) experimental hepatitis (Example 17), 1=dichloroethane+Acetaminophen, 2=dichloroethane+Acetaminophen+Legalon®, 3=dichloroethane+Acetaminophen+Essentiale®, 4=dichloroethane+Acetaminophen+GSSG·inosine;

FIG. 37 shows changes in cytolytic syndrome indices (ALT, AST) at combined experimental hepatitis (Dichlorethane+Acetaminophen) (Example 17), ROW1=ALT, ROW2=AST, 1=intact animals, 2=combined toxication, 3=treatment with GSSG·inosine, 4=treatment with Legalon®, 5=treatment with Essentiale®;

FIG. 38 shows changes in the blood bilirubin content at combined experimental hepatitis (Dichlorethane+Acetaminophen) (Example 17), ROW1=ALT, ROW2=AST, 1=intact animals, 2=combined toxication, 3=treatment with GSSG·inosine, 4=treatment with Legalon®, 5=treatment with Essentiale®;

FIG. 39 shows the cytokine serum in patients with chronic viral hepatitis C before and after treatment with GSSG·inosine (see Example 11);

FIG. 40 shows the development of immunologic indices in patients with acute viral hepatitis B treated with GSSG·inosine (see Example 5);

FIG. 41 shows the development of immunologic indices in patients with chronic viral hepatitis C before and after treatment with GSSG·inosine (see Example 11); and

FIG. 42 is a scheme outlining the synthesis of inosyl-5′-phosphoryl-GSSG-Na₂, I=inosine-5-monophosphate, HOSu=oxysuccinimide, DCC=dicyclohexylcarbodiimide, II=inoside-5-monophosphate oxysuccinimide activated ether, III=insoine-5-monophosphoryl-N-glutathione (Example 1).

### DETAILED DESCRIPTION

In one aspect, the present invention comprises an organic salt of oxidized glutathione (“GSSG”). Glutathione (GSH) comprises a glutamic acid residue bonded to a cysteine residue bonded to a glycine residue. A “residue” refers to a single unit within a larger molecule which is made up of two or more smaller molecules. The term “residue” accounts for the fact that upon bonding two molecules together to form a larger molecule, often a few atoms or small molecules (such as water) are removed. Oxidized glutathione is formed by oxidizing the —SH group of the cysteine residue in glutathione such that two —S· radicals combine to form a dimer linked by a disulfide unit. “Organic salt” refers to a salt where at least one counterion comprises an organic compound. In this aspect, the composition comprises a salt of GSSG, in which at least one counterion comprises a nitrogenous base. A “nitrogenous base” includes any nitrogen-containing molecule having the chemical properties of a base. In one embodiment, the nitrogenous base includes any derivatives of NH₃ in which the hydrogen atoms are substituted with organic residues.

Interaction of the nitrogenous base with compounds such as minerals or organic acids results in salt formation. Within an ionic pair, the nitrogenous base exists as a cation, due to interaction via a lone pair on the nitrogen atom of the nitrogenous base. One of ordinary skill in the art can screen nitrogenous bases and chemically interact the base with GSSG. Those that form stable organic salts are intended to be encompassed in this aspect of the invention.

Exemplary nitrogenous bases include DNA bases, nucleosides of DNA bases, nucleotides of DNA bases, RNA bases, nucleosides of RNA bases, nucleotides of RNA bases, inosine, nucleotides of inosine and homologues, analogues and derivatives thereof. The DNA bases are purines (adenine, guanine), and pyrimidines (thymine and cytosine). The RNA bases are purines (adenine and guanine), and pyrimidines (uracil and cytosine). A nucleoside useful in this invention comprises a nitrogenous base bonded to a sugar, examples of which are ribose or deoxyribose. Other examples of nucleosides includes the aldo- and ket-trioses, and corresponding tetroses and hexoses, as well as all stereoisomers. For example, aldohexose is known to have eight pairs of enantiomers including mannose, galactose, and glucose. In another embodiment, a suitable sugar in accordance with this aspect of the invention includes any sugar having similar chemical reactivity as glucose. A nucleotide comprises a nitrogenous base, a sugar and one or more phosphate groups. Alternatively, a nucleotide comprises a nucleoside and one or more phosphate groups. In one embodiment, the nucleotide comprises a monophosphate, a diphosphate, or a triphosphate.

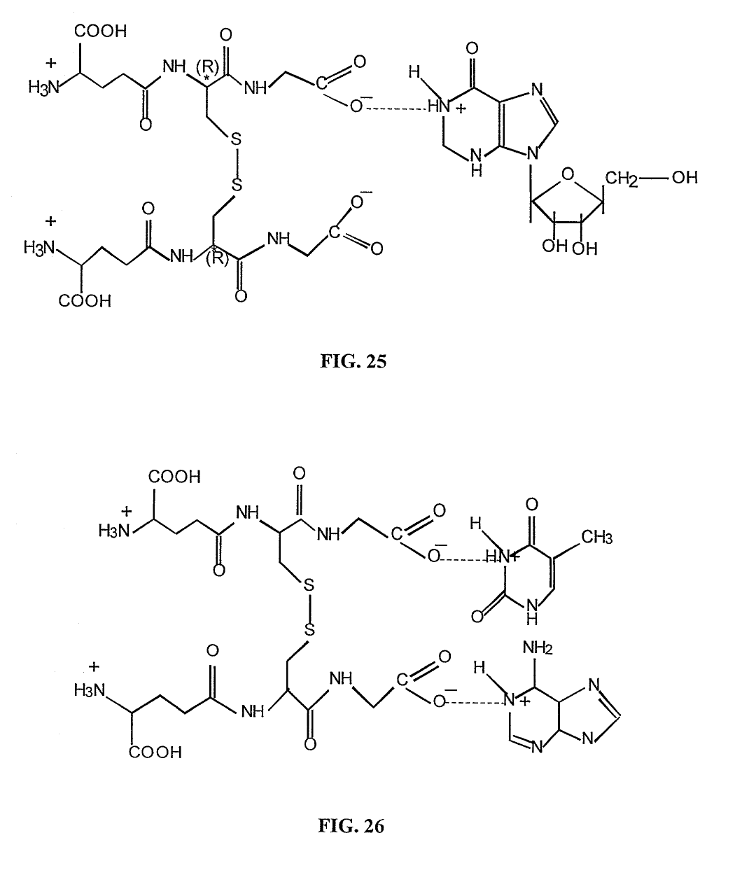

The cysteine or glutamic acid residues in GSSG can exist in either the D- or L-form. In one embodiment, all amino acid residues in GSSG exist in the L-form. In another embodiment, two chemically equivalent amino acids exist in the D-form. For example, both cysteine residues exist in the D-form or both glutamic acid residues exist in the D-form, and the remainder of the amino acids exist in the L-form. A preferred embodiment provides the D-form of GSSG·inosine having two chemically equivalent amino acids (cysteines) in D-form, and other amino acids are in L-form (see FIG. 25). In another embodiment, the D-GSSG-uracyl formulation given in the FIG. 24 provides glutamic acid in the D-form.

The oxidized glutathione has multiple sites that can include a cationic or anionic residue. The charge on one residue may not necessarily dictate the overall charge of the oxidized glutathione, as other sites may augment the charge, neutralize the charge or have no effect at all. For example, the presence of one or more anionic residues may result in a negative overall charge of GSSG, although the presence of one or more cationic residues may result in a neutral or positively charged molecule. In one embodiment, the oxidized glutathione comprises an anionic residue. Preferably, the anion is formed by deprotonation at any site containing a proton. For example, deprotonation of carboxylic acids results in negatively charged carboxylate groups, either on one or more of the glycine residue and/or on one or more of the glutamic acid residues.

An anionic residue requires a positively charged counterion. In one embodiment, the counterion comprises a nitrogenous base comprising a protonated nitrogen atom. In another embodiment, the oxidized glutathione comprises a cationic residue. Preferably, the cation is formed by a protonation reaction. For example, protonation at any of the nitrogen atom sites can result in a positively charged ammonium group.

It is understood that the oxidized glutathione, in addition to the organic counterion, can also have other counterions, including the same or another type of organic counterion or inorganic counterions including ammonium cations (having one or more non-hydrogen groups), alkaline metals, alkaline earth metals and transition metals. Where the oxidized glutathione has more than one organic counterion, in one embodiment one counterion comprises a purine and the other comprises a pyrimidine. In another embodiment, both counterions can be the same or a different type of purine, or both counterions can be the same or a different type of pyrimidine. Alternatively, the counterion can comprise the corresponding nucleosides or nucleotides of the aforementioned purine or pyrimide.

In one embodiment, the nitrogenous base is inosine, i.e. 9-β-D-ribofuranosyl-hypoxanthine (inosine) having a schematic structural formulation as shown in FIG. 9. Preferably, the salt comprises oxidized glutathione and a counterion comprising inosine in a molar ratio of about 1:1. It has been discovered that this ratio provides a particularly enhanced biological and pharmacological effect. Another surprising discovery is that inosine, being only slightly soluble in aqueous solution, becomes easily soluble as a counterion in an organic salt of GSSG (“GSSG·inosine”). Without wishing to be bound by any theory, this change in solubility is evidence for the formation of an ionic bond between GSSG and inosine, i.e. formation of an organic salt.

In one embodiment, the nitrogenous base comprises a homologue of DNA bases, nucleosides of DNA bases, nucleotides of DNA bases, RNA bases, nucleosides of RNA bases, nucleotides of RNA bases, inosine, or a nucleotide of inosine. “Homologue” as used herein refers to a composition belonging to a chemical series whose successive members have a regular difference in composition. For example, homologues can differ from a parent composition by one or more methylene groups. Preferred homologues give a desired therapeutic effect.

In one embodiment, the nitrogenous base comprises an analogue of DNA bases, nucleosides of DNA bases, nucleotides of DNA bases, RNA bases, nucleosides of RNA bases, nucleotides of RNA bases, inosine, or a nucleotide of inosine. “Analogue” as used herein includes a chemical compound that is structurally similar to the nitrogenous base but differs slightly in composition (as in the replacement of one atom by an atom of a different element or in the presence of a particular functional group). An example of an analgue of a nitrogenous base includes 4-thiouracil, in which an oxygen atom of uracil is replaced with a sulfur atom. Preferred analogues give a desired therapeutic effect.

In one embodiment, the nitrogenous base comprises a derivative of DNA bases, nucleosides of DNA bases, nucleotides of DNA bases, RNA bases, nucleosides of RNA bases, nucleotides of RNA bases, inosine, or a nucleotide of inosine. “Derivative” as used herein includes nitrogenous bases derivatized with groups such as alkyls, aryls, amines, thiols, phosphates and amino acids. Example derivatives include 5-methyl-cytosine, and dihydrouracil. One of ordinary skill in the art can readily use methods, such as combinatorial methods, to prepare appropriate derivatives of nitrogenous bases which will form desired salts with oxidized glutathione. In another embodiment, a derivative of a nitrogenous base involves modification at an amide or carboxylate group. Preferred derivatives give a desired therapeutic effect.

In one embodiment, the GSSG can be modified at an amide or carboxyl group.

Another aspect of the present invention provides a composition comprising an oxidized glutathione covalently bonded to inosine.

In one embodiment, the oxidized glutathione is covalently bonded to inosine via a phosphoramide linkage. In one emodiment, a ratio of the oxidized glutathione to inosine is 1:1. The inosine can also be provided as either a nucleoside or nucleotide of inosine.

Another aspect of the invention provides a method of making an organic salt. The method comprises the step of providing a solution comprising glutathione. In one embodiment, the oxidized glutathione is produced by adding glutathione to a basic solution. To obtain an organic salt, it is generally desired that both components (acid and base) are at least partially ionized. In an embodiment, the solution has a pH as shown in the following equations (1) and (2):

pH≧pKₐ−2 (1)

pH≦16−pK_{b} (2)

Equations (1) and (2) take into account the ionization constants of GSSG (pKₐ) and the corresponding nitrogenous base (pK_{b}).

Glutathione can then be oxidized to form the cysteine residue radical that recombines with another like radical to form the disulfide bond. The oxidation can occur either electrochemically or chemically. Chemical oxidizing conditions can involve the addition of an oxidant. Examples of oxidants include hydrogen peroxide, air or oxygen plus a catalyst as known in the art, iodine, nitrogen oxides and sulfur compounds having an active oxygen such as dimethylsulfoxide. “Active oxygen” refers to an oxygen atom capable of being transferred to another compound. Any nitrogenous base, as disclosed herein, can be added to the solution.

Alternatively, an organic salt can be prepared by providing the oxidized glutathione itself in a solution in anionic form. In this embodiment, the glutathione is first oxidized followed by addition to a basic solution. In one embodiment, the solution has a pH as defined by equations (1) and (2).

Another aspect of the invention provides a method of treating an infectious disease disease via an agent comprising a GSSG salt comprising at least one counterion comprising a nitrogenous base. Exemplary classes of infectious diseases include viral infectious diseases, bacterial infectious diseases, anaerobic infections, chlamydia infections, mycoplasma infections, mycoses and protozoa infections. The present compositions are preferably to be used as therapeutics for the treatment of a wide range of infectious diseases and for the prevention of their complications. Preferably, the targeted infectious diseases are those in which the progression of the disease displays:

a) intracellular persistence at the infecting agent life cycle;

b) quasi-variable genome of the infecting agent enabling the latter to escape the host immune surveillance;

c) altered reasonable balance (regarding the host defense) of the cytokine production by the Th1/Th2 groups of lymphocyte-helpers, i.e. altered directivity of the effective host immune surveillance;

d) systemic cytopathic effects, particularly, hepato-, nephro- and hemocytopathic effects.

The method takes into account various strategies including stimulation of T-cell and/or humoral anti-infectious immunity, ensuring cell-protective effects. Apoptosis mechanisms can be induced, including expression of an apoptotic inducer, FAS/APO-1 antigen (CD95⁺), in the virus-infected cells.

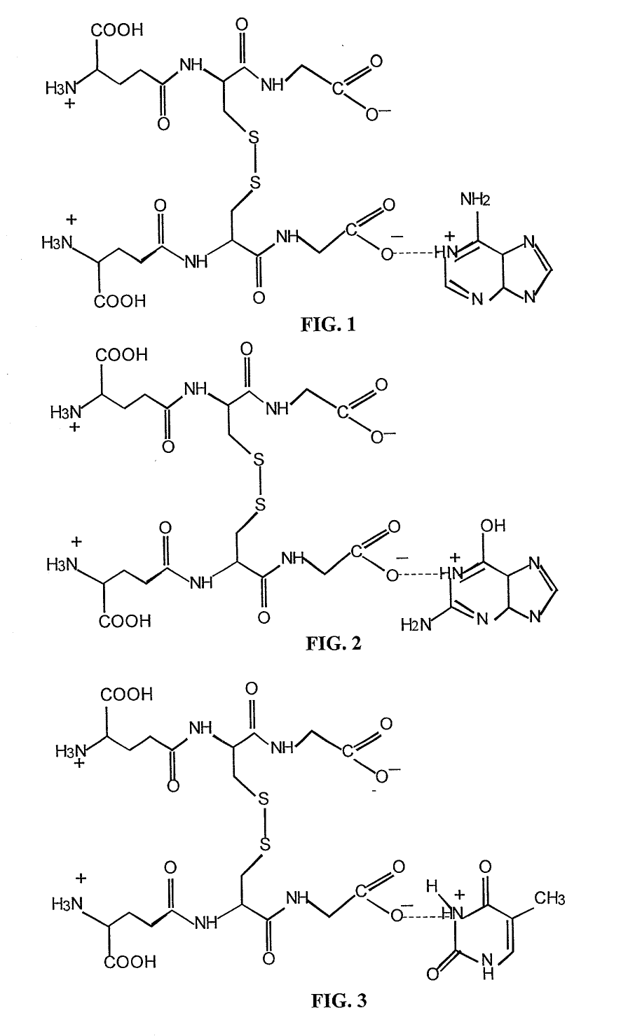

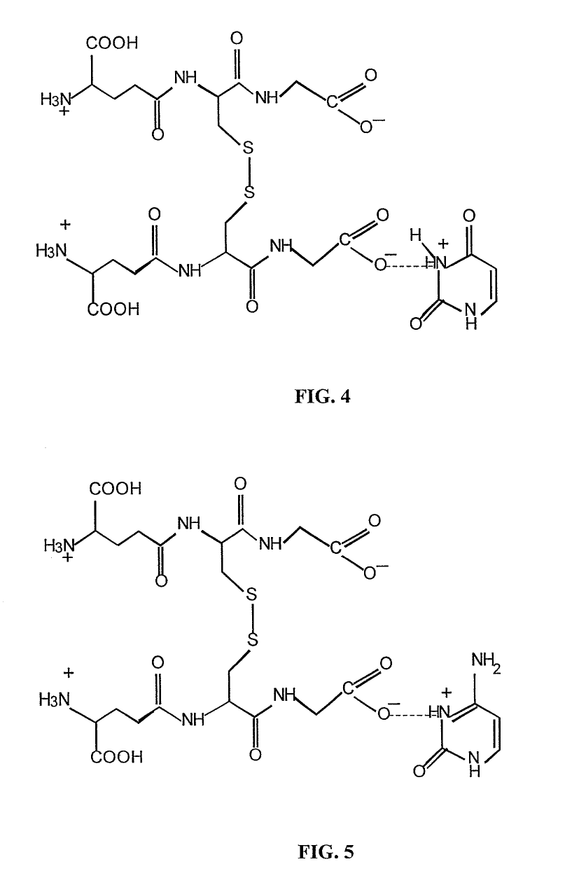

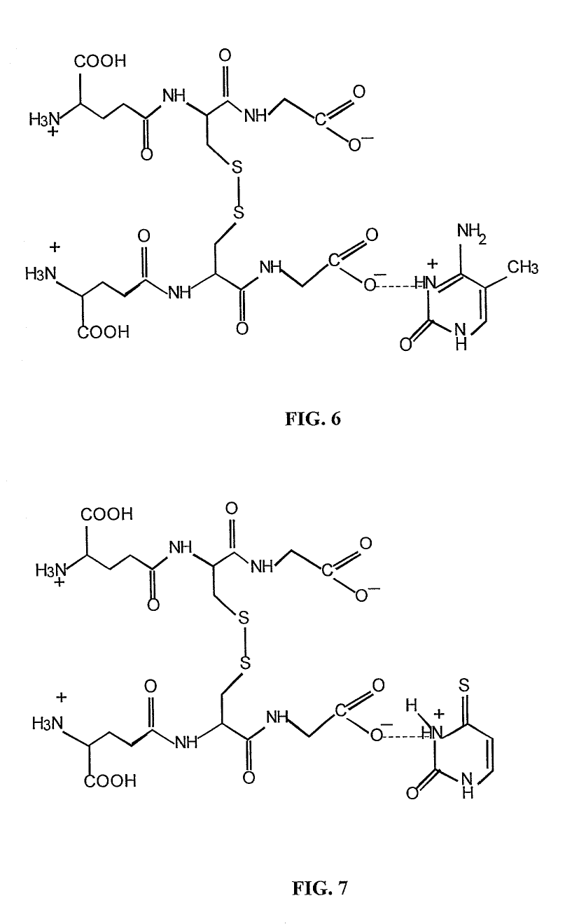

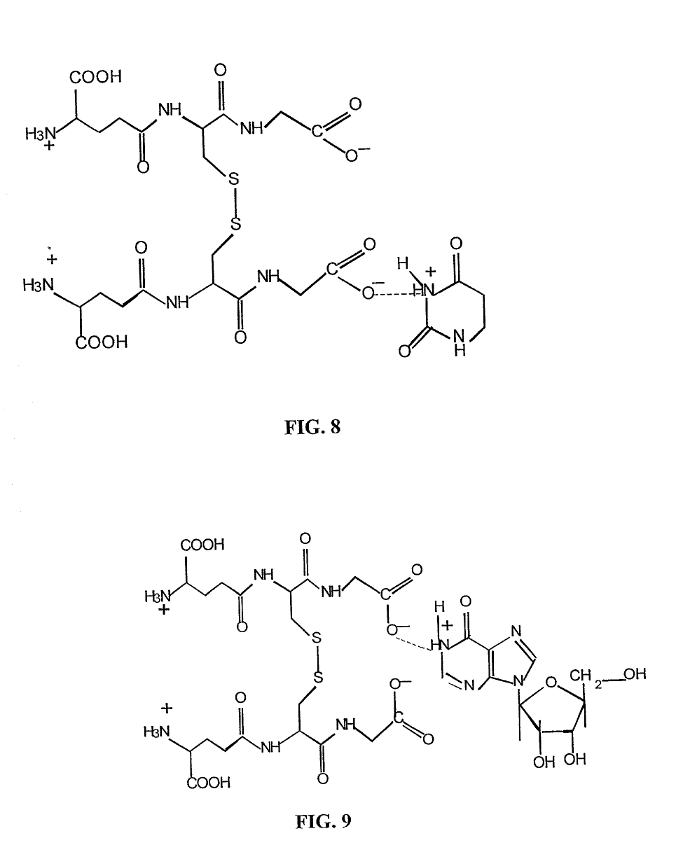

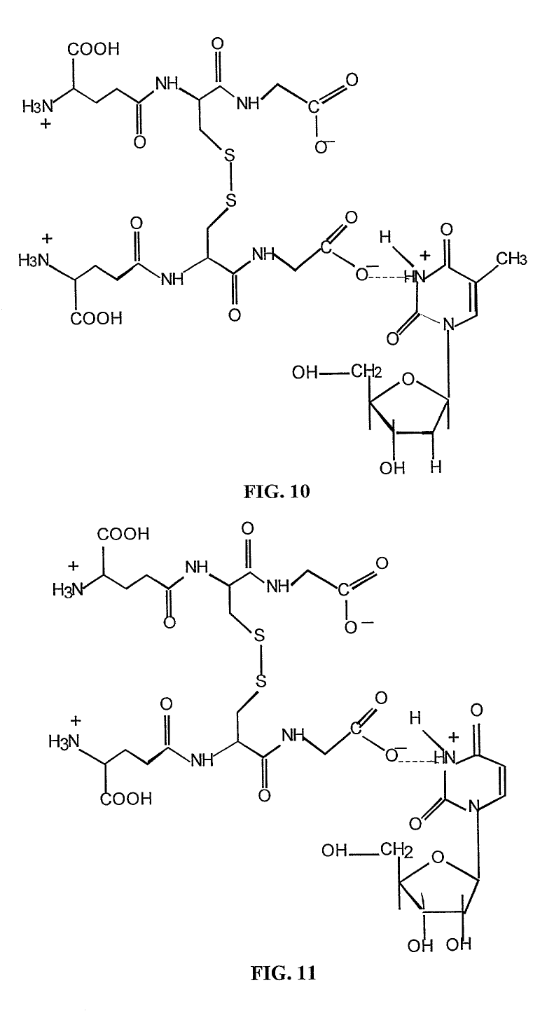

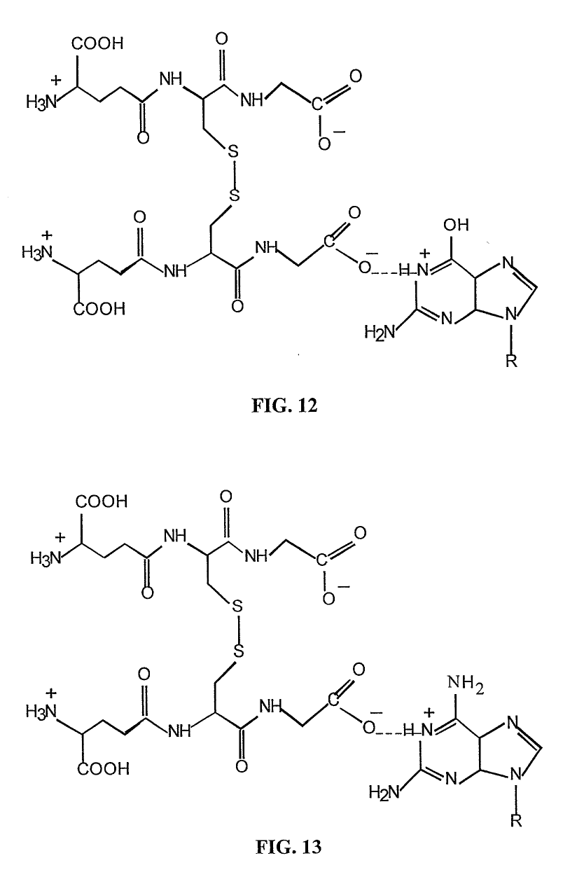

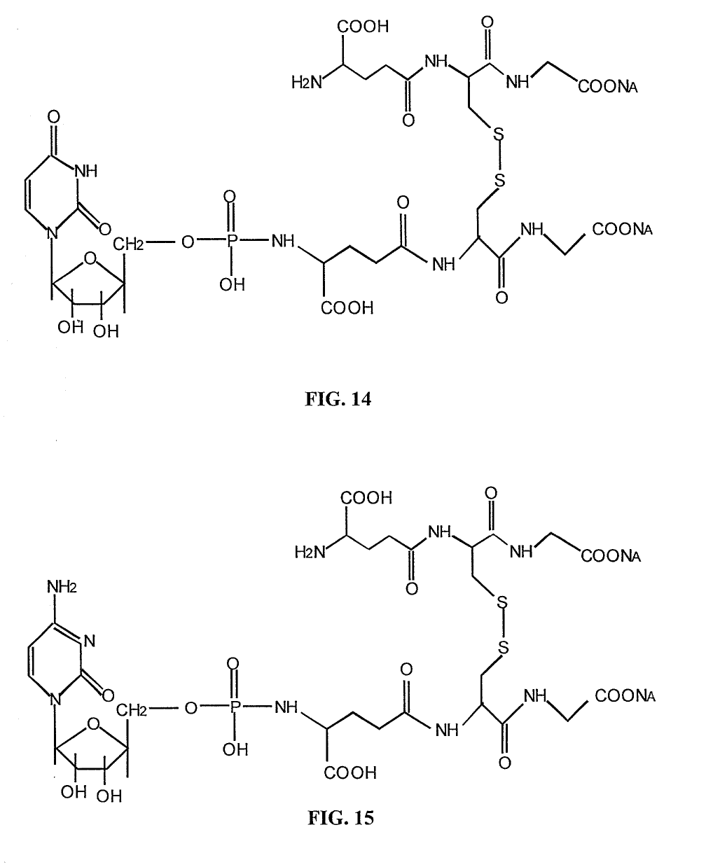

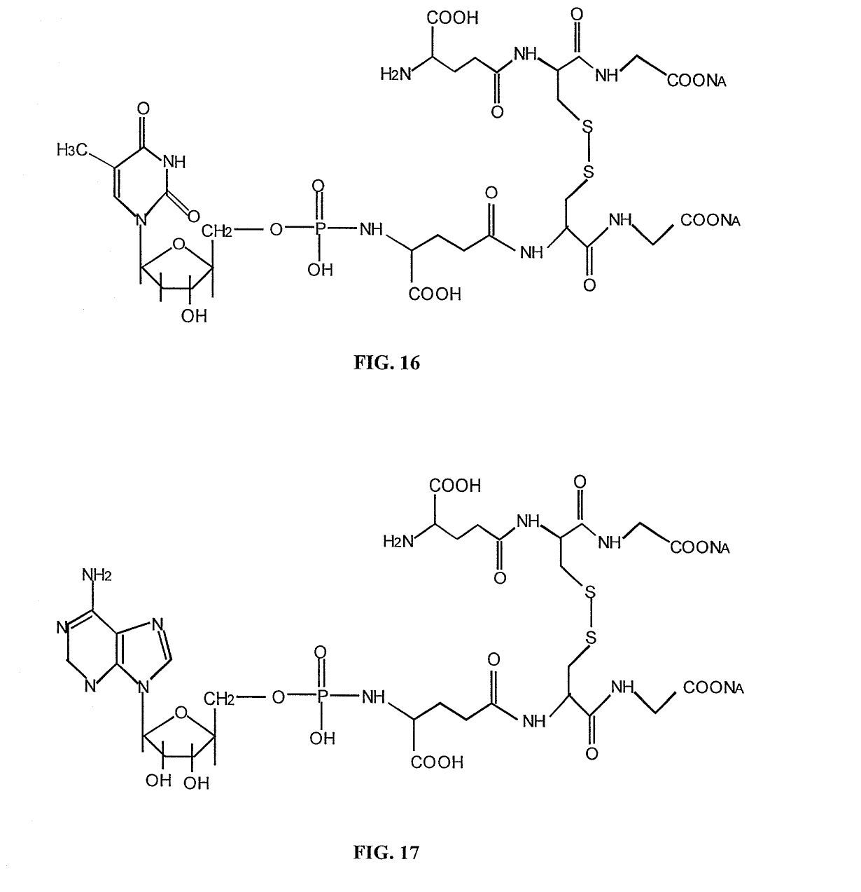

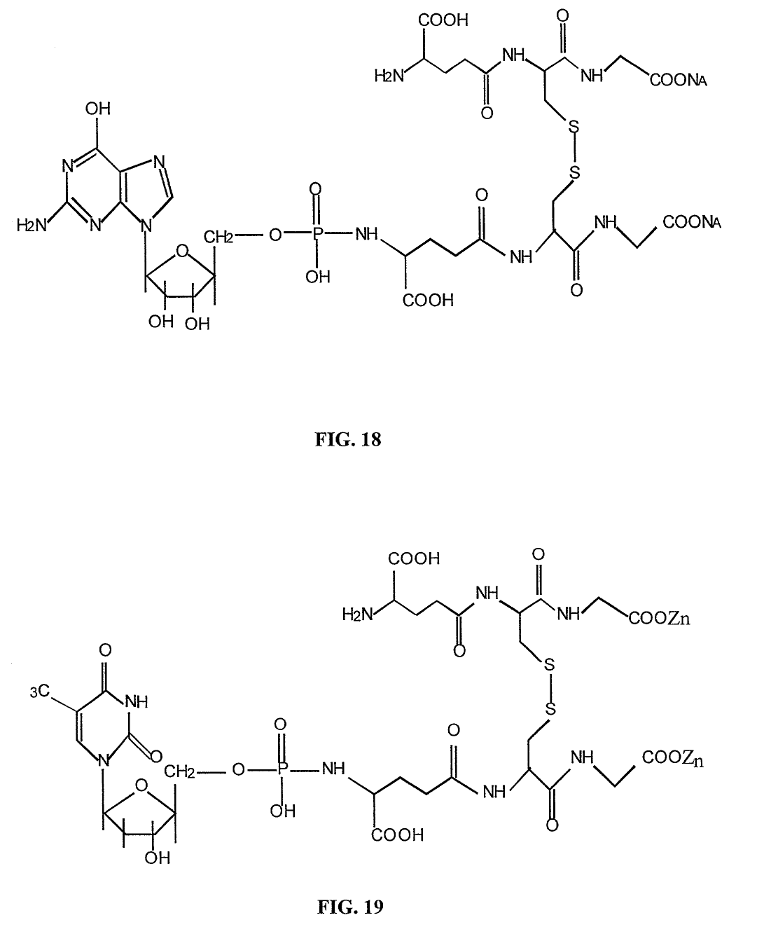

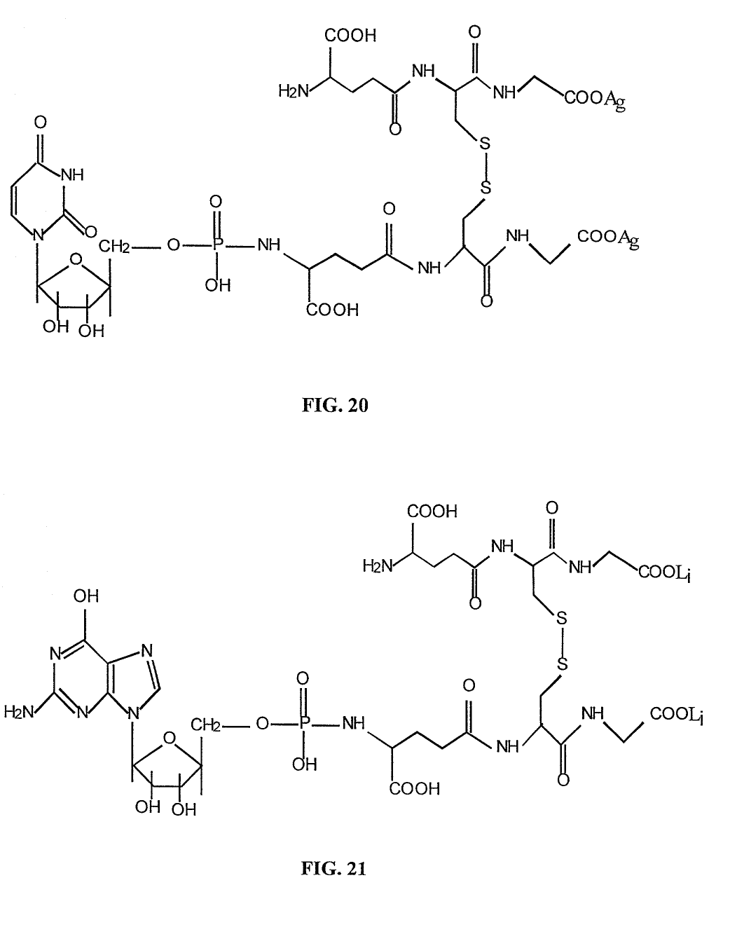

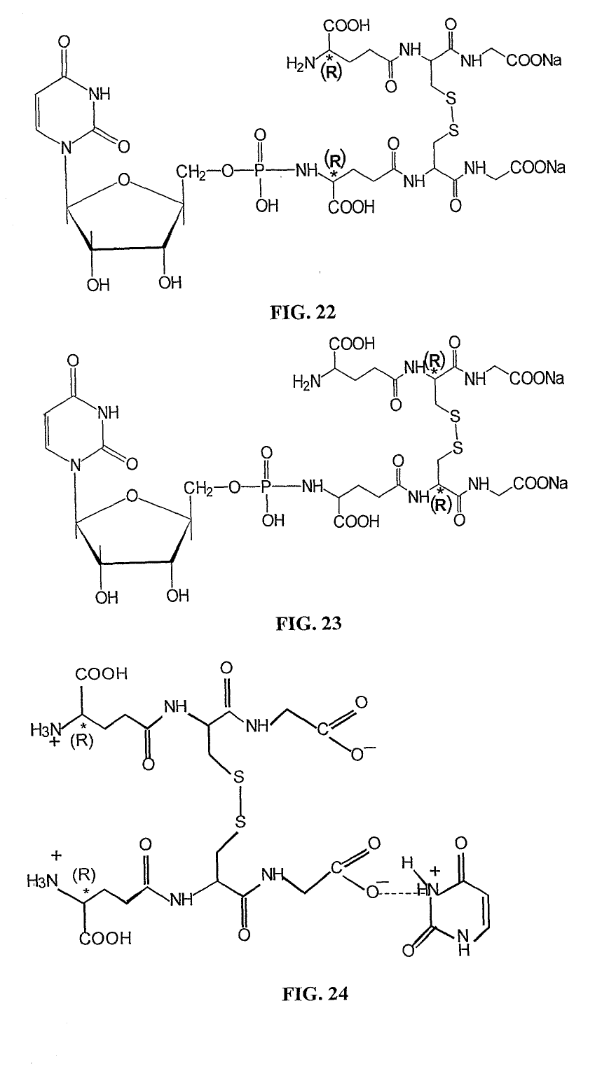

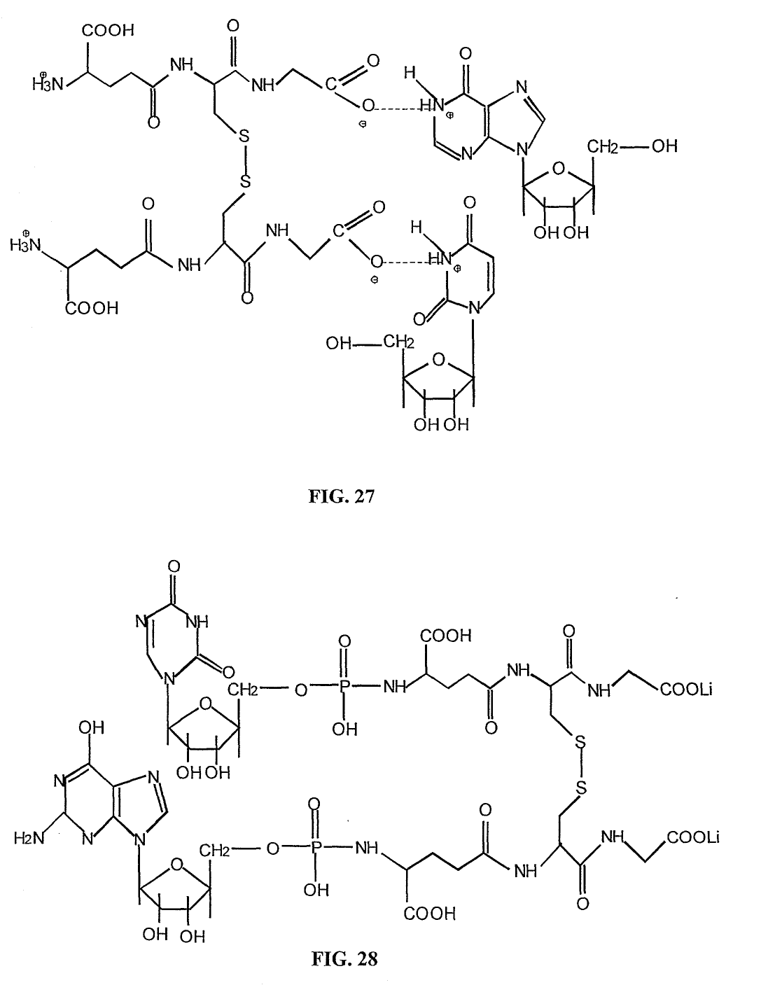

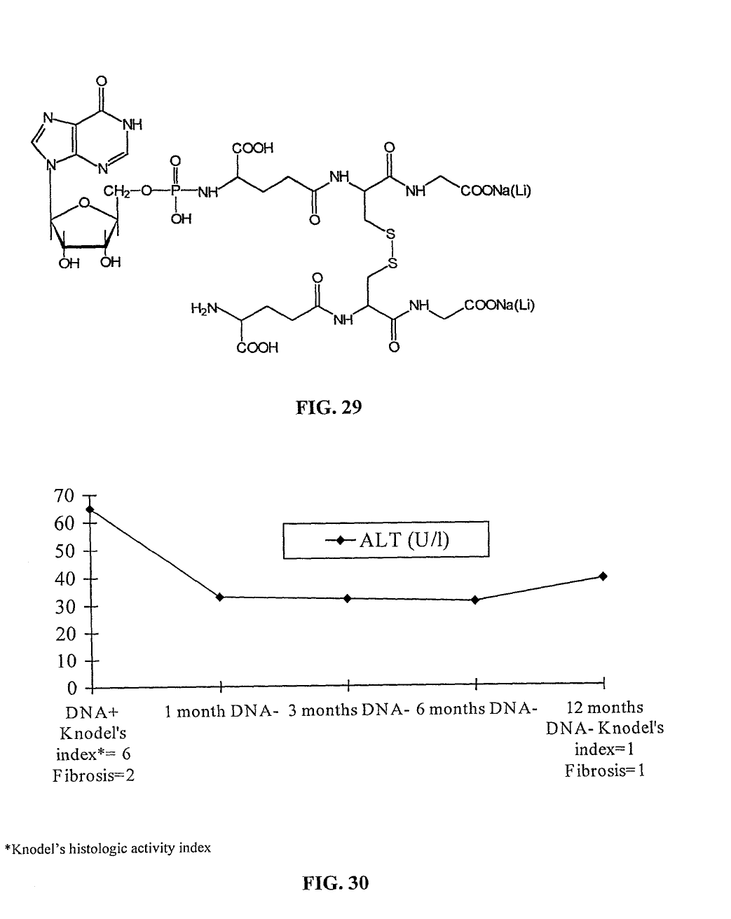

The compounds disclosed herein (see formulations—FIGS. 1-29) possess a unique combination of the following biological and pharmacological effects: a) direct (inhibition of the HCV NS3 ATP-ase/helicase activity) and indirect (through apoptosis induction in the virus-infected cells including expression of an apoptotic inducer, the FAS/APO-1 receptor) antiviral activity; b) immunorehabilitating activity; and c) systemic cell-protective and, particularly, specific hepatoprotective effects capable of cirrhotic liver alteration prevention and even reverse of the liver fibrosis.

It is to be understood that any one or more of the compounds of the invention can be used in combination to treat any one or more of the diseases disclosed herein.

In one embodiment, where the disease involves macrophages containing tuberculosis mycobacteria, or cells infected with mycoplasma, chlamydia, malaria plasmodium and other infecting agents, the preferred treatment agent is an effective amount of a composition selected from the group consisting of GSSG-inosine, GSSG-uracil, GSSG·thymine, GSSG-adenosine, GSSG-guanine, GSSG-inosine-monophosphate, GSSG-uracil-monophosphate, GSSG-thymidine-monophosphate and GSSG-cytosine-monophosphate.

In one embodiment the subject is infected with hepatitis C virus, and the composition comprises at least one compound selected from GSSG·inosine, GSSG-inosine-monophosphate and GSSG-uracil-monophosphate.

In one embodiment, the subject is infected with hepatitis B and/or hepatitis C and the composition is GSSG·inosine and/or GSSG-inosine-monophosphate.

In one embodiment, the subject is infected with acute viral hepatitis B and the composition is GSSG·inosine and/or GSSG-inosine-monophosphate.

In one embodiment, the subject is infected with, chronic hepatitis B, chronic hepatitis C, toxic hepatitis, post-alcoholic liver disease, liver cirrhosis, hepatocellular carcinoma and combinations thereof, and the composition is GSSG·inosine.

In one embodiment, the subject is infected with acute viral hepatitis C, and the composition is any one of GSSG·inosine, GSSG·uracil and GSSG-inosine-monophosphate.

In one embodiment, the subject is infected with chronic viral hepatitis B, and the composition is any one of GSSG·inosine, GSSG·adenosine, GSSG·guanosine, GSSG-inosine-monophosphate and GSSG-thymidine-monophosphate.

In one embodiment, the subject is infected with chronic viral hepatitis C, and the composition is any one of GSSG·inosine, GSSG·uracil, GSSG·cytosine, GSSG·dihydrouracil, GSSG-uracil-monophosphate, GSSG-cytosine-monophosphate and uracil-GSSG-inosine.

In one embodiment, the subject is infected with chronic viral hepatitis in cirrhotic stage, and the composition is any one of GSSG·inosine, GSSG·uridine, GSSG·ethymidine, Li₂-GSSG-inosine-monophosphate and Na₂-GSSG-thymidine-monophosphate.

In one embodiment, the subject is infected with lung tuberculosis, and the composition is any one of GSSG·inosine, GSSG·cytosine, GSSG-5-methylcytosine and Li₂-GSSG-inosine-monophosphate.

In one embodiment, the subject is infected with urogenital tuberculosis, and the composition is any one of GSSG·thymine, Na₂-GSSG-guanosine-monophosphate and uracil-Li₂-GSSG-guanosine-monophosphate.

In one embodiment, the subject is infected with any one or any combination of AIDS, cytomegalovirus infection, infection caused by Epstein-Barr virus and infection caused by pneumocysts, and the composition is any one of GSSG·inosine, GSSG·dihydrouracil, GSSG-4-thiouracil, Zn₂-GSSG-thymidine-monophosphate, Ag₂-GSSG-uracil-monophosphate and uridine·GSSG·inosine.

In one embodiment, the subject is infected with herpetic infection, and the composition is any one of GSSG·inosine, Li₂-GSSG-guanosine-monophosphate, the D-form of Na₂-GSSG-cytosine-monophosphate and the D-form of GSSG·uracil.

In one embodiment, the subject is infected with candidiasis, and the composition is any one of GSSG·uridine, GSSG-4-thiouracil and Ag₂-GSSG-uracil-monophosphate.

In one embodiment, the subject is infected with mycoplasma infection, and the composition is any one of GSSG·inosine, GSSG·adenosine and Na₂-GSSG-adenosine-monophosphate.

In one embodiment, the subject is infected with chlamydia infection, and the composition is any one of GSSG·inosine, GSSG·thymine, GSSG·uridine, GSSG·guanosine and Na₂-GSSG-guanosine-monophosphate.

In one embodiment, the subject is infected with any one of malaria and leishmaniasis, and the composition is any one of GSSG·inosine, GSSG·cytosine and GSSG-5-methylcytosine.

In one embodiment, the subject is infected with an anaerobic infection, and the composition is any one of the D-form of GSSG·inosine (D-cysteine) and the D-form of GSSG·uracil (D-glutamic acid).

In one embodiment, the subject is infected with any one of viral hepatitis A, dysentery and cholera, and the composition is any one of GSSG·inosine, GSSG-inosine-monophosphate and the D-form of GSSG·uracil (D-glutamic acid).

In one embodiment, the subject is infected with infectious meningitis, and the composition any one of GSSG·inosine, Li₂-GSSG-inosine-monophosphate, the D-form of GSSG·uracil (D-glutamic acid), GSSG-5-methylcytosine and Ag₂-GSSG-uracil-monophosphate.

In one embodiment, the subject is infected with any one of the plague, tularemia and anthrax, and the composition is any one of GSSG·inosine, GSSG·adenine, GSSG·thymine, GSSG-5-methylcytosine, GSSG-4-thiouracil, the D-form of GSSG·uracil (D-glutamic acid), the D-form of GSSG·inosine (D-cysteine) and adenine·GSSG·thymine.

In one embodiment, the subject is infected with an infection caused by prions, and the composition is any one of GSSG·inosine, GSSG·uridine, GSSG·dihydrouracil, Ag₂-GSSG-uracil-monophosphate, Ag₂-GSSG-thymidine-monophosphate and uracil-monophosphate-Li₂-GSSG-inosine-monophosphate.

In one embodiment, the subject is infected with any one of the flu and acute respiratory infections, and the composition is any one of GSSG·inosine, GSSG·adenosine, GSSG·uracil and GSSG·thymine.

Another embodiment of the invention provides a therapeutic agent comprising GSSG·inosine. It is a surprising discovery that GSSG·inosine possesses a unique combination of biological and pharmacological effects in the treatment of the following diseases: viral hepatitis B and C and complications thereof, AIDS (see Examples No. 5-8, 12-13); herpetic and urogenital infections (see Examples No. 14-15). According to this aspect of the invention, beneficial therapeutic efficacy of GSSG·inosine, particularly for viral hepatitis, has been found effective for 3 types of activity, including antiviral, immunorehabilitating and hepatoprotective activities.

Each of three GSSG·inosine pharmacological activity types was found to be unique. Without wishing to be bound by any theory, various mechanisms can be contemplated, including antiviral, immunorehabilitating and hepatoprotective activities of GSSG·inosine (the D-forms thereof) and other compounds obtained through disulfide-containing peptides and purines/pyrimidines interaction.

One antiviral mechanism involves inducing apoptotic mechanisms in virus-infected cells, among others, including enhanced expression of apoptotic inducer, FAS/APO-1-receptor (CD95+) (see Example No. 3). This mechanism involves high efficacy of GSSG-inosine and some other compounds (GSSG-IMP, GSSG-UMP, Li₂-GSSG-IMP, Zn₂-GSSG-TMP) for treatment and prevention of diseases caused by DNA and RNA viruses.

It is another discovery that GSSG·inosine antiviral activity was displayed in several types of viral infections such as: Rift valley fever (RVF), generalized herpetic infection; Venezuelan horse encephalomyelitis (VHE); flu (type A virus, H3N2) (see Example No. 2), suggesting that GSSG-inosine is more effective than conventional antiviral agents.

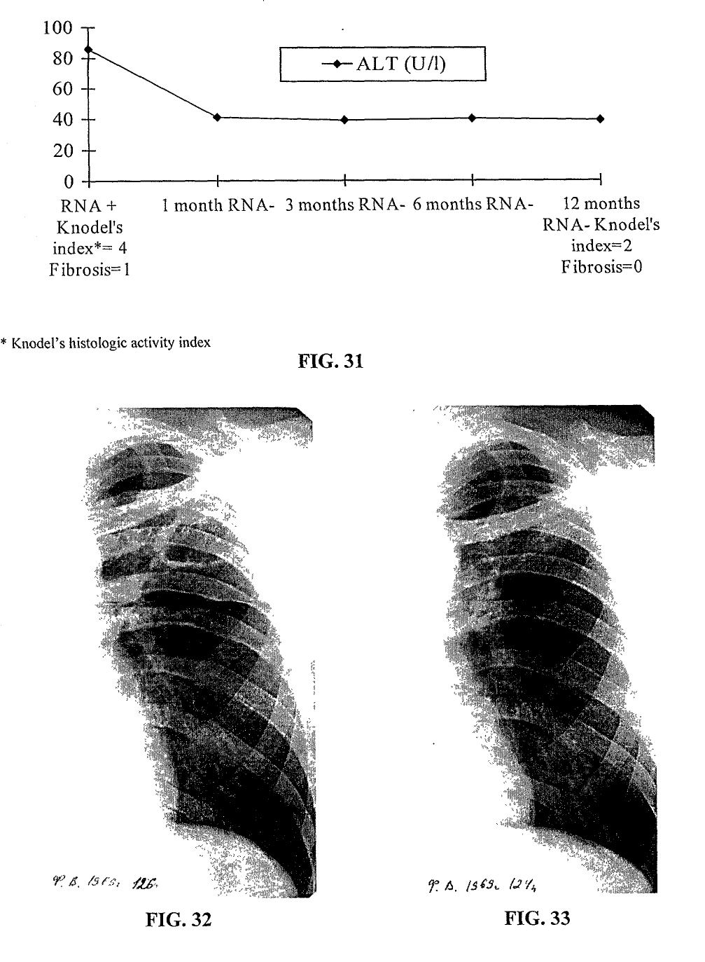

Moreover, specific therapeutic activity of GSSG·inosine and other disclosed compounds was shown at an infectious disease clinic for the treatment of tuberculosis (see Example 16); urogenital infections (see Examples 14-15); AIDS (see Examples 12 and 13); acute and chronic hepatitis B (see Examples 5-8, Tables 12-21, and FIGS. 30 and 31).

Thus, an advantageous feature of GSSG·inosine and other disclosed compounds to induce apoptosis in the virus-infected cells also involves cells infected with tuberculosis mycobacteria, chlamydia, mycoplasma, ureaplasma and other infecting agents.

The therapeutic efficacy of, for instance, GSSG·inosine, for acute and chronic hepatitis B was displayed by restoration of transaminase activity (ALT, AST), bilirubin, prothrombin and HbsAg content, which was shown to be significantly higher than therapeutic efficacy of conventional treatment.

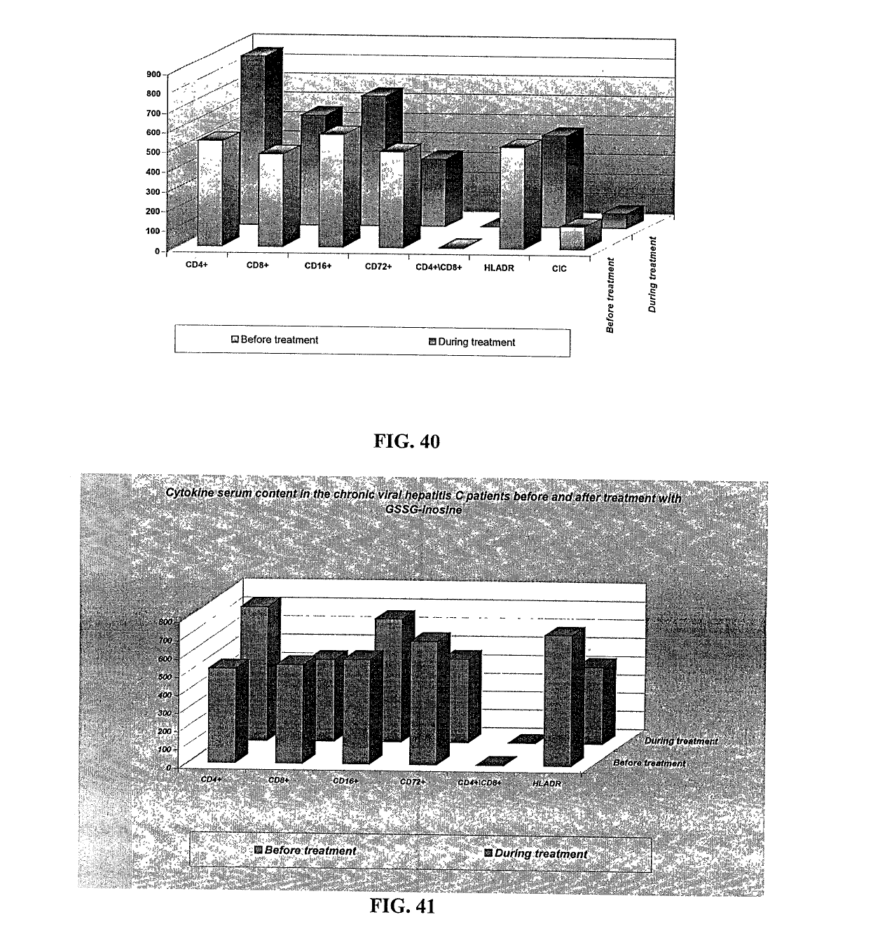

For example, in the case of acute hepatitis B, the conventional treatment monitors positive changes of transaminase activity, bilirubin and HbsAg content via a 50-60 day therapy. By applying GSSG·inosine as a single-agent therapy, the results are attained by 14-17 days (see Examples 5 and 6, Tables 12-17, and FIG. 40).

In the case of chronic hepatitis B, an indication of applied treatment efficacy is transaminase activity (indicating cytolysis rate of hepatocytes) and replicative viral activity determined by PCR assay. Using this index, chronic hepatitis B treatment efficacy with conventional agents (recombinant interferons and nucleotide analogues) provide a percentage decrease of positive PCR on HBV DNA (from 100%) by not more than by 30-40%, i.e. the therapy in 60-70% of the patients is not effective. In contrast, applying GSSG-inosine results in positive (in 100% of the cases) HBV DNA PCR conversion into negative in 75-90% of the patients (see Example No. 7-8+Tables No. 17-21+FIGS. 30 and 31). Restoration of functional liver capacities in case of viral and toxic hepatitis determined through biosynthesizing and detoxicating indices can be achieved by applying GSSG·inosine in a time period reduced by a factor of 2-3 compared to a treatment with, for instance, Heptral® (S-adenosyl-methionine, Knoll GmbH, Ludwigshafen, Germany) or Essentiale® (Aventis Pharmaceuticals Inc., Bridgewater, N.J., see Example No. 11, 14).

Another embodiment features specific antiviral activity of GSSG·inosine, GSSG-IMP and GSSG-UMP as well as the salts thereof in the treatment of hepatitis C virus (HCV RNA), in contrast to the DNA-containing hepatitis B virus (HBV DNA). These preferred agents are capable of inhibiting ATP-ase/helicase activity of regulatory non-structural protein—NS3 of hepatitis C virus (see Example No. 4). Zn₂-GSSG-IMP and other salts of GSSG-UMP such as Ag₂-GSSG-UMP can inhibit activity of the non-structural (regulatory) enzyme-proteins of both HCV and HIV.

Potent therapeutic efficacy of GSSG·inosine for acute and chronic viral hepatitis, herpes and AIDS (see Examples No. 5-14) can be realized with a combination of indirect (apoptosis induction in the virus-infected cells) and direct antiviral properties (inhibition of the HCV NS3ATP-ase/helicase activity), which is a unique feature for preventive and therapeutic actions for viral hepatitis C.

Simultaneously, the GSSG·inosine D-form exhibited beneficial antiviral activity for viral infections, especially viral hepatitis, and also for AIDS, ensuring cessation of the high viral replicative activity and their elimination (see Examples No. 12).

Direct activation of T-cell immunity via the GSSG-inosine immunorehabilitating features was found to be very important in treatment of viral hepatitis and, in particular, hepatitis C, as a second type of the pharmacological activity of GSSG·inosine and generic compounds.

Characteristic features of the GSSG-inosine immunorehabilitating efficacy are the following:

normalization of cytokine production ratio by Th1/Th2 cells with prevailing activation of T-cell immunity;

prevailing production stimulation of IL-2, IL-12 as well as IFN and TNF.

The clinical case Examples No. 9-11 show that the altered cytokine production balance by the Th1/Th2 T-helper groups characteristic for a severe course to chronic infection (unfavorable chronic hepatitis C course) is beneficially restored upon application of GSSG·inosine. Moreover, the prevailing activity of the Th2 cytokine group such as IL-4, IL-10, IL-6 and IL-13 observed in the HCV-infected patients after the GSSG·inosine administration transforms into prevalence of the Th1 cytokine group activity with enhanced release of IL-2, IL-12, IFN-α and γ and TNF-α and β. Simultaneously the T-cell immunity is considerably boosted (CD4+, CD8+, CD16/56+, CD25+ counts are increased, resident macrophages are stirred up) ensuring that HBV and HCV (viruses of the B and C hepatitis) are brought under proper immunologic surveillance and, thereupon, eliminated resulting in favorable therapeutic outcome.

One important mechanism of the GSSG·inosine therapeutic effects is induction of the IL-12 release that facilitates maintenance of the Th1 group activity prevalence and, therefore, active production of endogenous (host own) interferons (see Examples No. 5-7, 12).

Another type of the GSSG·inosine biologic and pharmacological activity as an agent is the hepatoprotective activity that purposefully determines unique anti-cirrhotic effects. Infectious diseases which display this mechanism are viral hepatitis, particularly, hepatitis C.

40% of the liver cirrhosis and 60% if the liver cancer are caused by chronic hepatitis C. Any constructive solution for the chronic hepatitis C treatment matter would likely imply both elimination of viral cytopathic impairment of the liver tissue and prevention of the liver fibrosis-formation processes along with prevention of the liver malignant transformation.

This aspect of the invention discloses rationale of the GSSG·inosine anticirrhotic effects obtained on experimental model of liver cirrhosis and in the patients. Particularly, the model of cirrhosis induced by chronic introduction of dimethylnitrosamine (DMNA) exhibited that the GSSG·inosine application dosing 10 mg/kg 3 times a week for 6 weeks ensured decreased amount of connective tissue by 64% and facilitated restoration of impaired hepatocytes. The comparison agent, Heptral® (S-adenosyl-methionine) showed significantly lower therapeutic effects decreasing the liver fibrosis-formation rate in the experimental animals only by 35% and having no considerable action on restoration of the liver function (protein synthesis, etc.) (see Examples No. 18).

Similar results were observed in the GSSG·inosine-treated patients with diagnosed chronic viral hepatitis B (and/or chronic hepatitis C); cirrhotic stage (PCR HBV+; PCR HCV+), ascites, portal hypertension. Several cycles of GSSG·inosine treatment administered as a single-agent therapy ensured unique therapeutic effect confirmed with positive development of the patients' clinical state and specific tests (see Examples No. 8-9, 11).

Thus, treatment of the patients with viral hepatitis, especially hepatitis C by applying GSSG·inosine ensures not only elimination of the very infectious process but also prevention of the viral hepatitis complications, particularly liver cirrhosis and cancer. Moreover, in the case of developed toxic cirrhosis that are similar to alcoholic cirrhosis, the GSSG·inosine application in experiments and in the patients exhibited therapeutic efficacy that has no match among known hepatoprotective therapeutics.

Further, the experimental and clinical studies (see Examples No. 2-18) showed that GSSG·inosine and other disclosed medicinal agents possess high therapeutic efficacy due to capability to correct aforementioned factors of etiopathogenesis of the infectious processes. Thus, GSSG salts and other therapeutic agents, as described in this application, can be used to provide a beneficial therapeutic effect in the following cases of infectious diseases:

For the treatment of infectious diseases where an etiotropic factor is an intracellular pathogen such as DNA and RNA viruses; chlamydia, mycoplasma and ureaplasma; for the treatment of acute and chronic viral hepatitis, AIDS, mycoplasmic and chlamydia infections.

For the treatment of protozoa infections (malaria, leishmaniasis);

For the treatment of disease caused by fungi (mycoses) and pneumocysts combined with conventional antibacterial treatment in the acute period and as a single-agent therapy for a supportive treatment.

For the treatment of infectious diseases caused by gram-positive and gram-negative bacteria as well as non-spore-forming anaerobes where etiopathogenesis is characterized with intracellular persistence of the infecting agent, for instance, tuberculosis (mycobacteria persistence in macrophages). In this case the apoptosis induction in the infected cells (including the macrophages) by the agents of the GSSG-nucleoside/nucleotide group ensures effective impact of the specific chemotherapy and T-cell immunity on the mycobacteria.

A “subject”, as used herein, refers to any mammal (preferably, a human) that may be susceptible to a condition associated with an infectious disease (such as the conditions described above).

In some aspects, the invention intends to treat subjects who are at risk of being infected with an infectious disease. These subjects may or may not have had a previous event related to an infectious disease. This invention embraces the treatment of subjects prior to the disease event, at a time of the disease event, following a disease event, or who have been diagnosed as having an infectious disease. Thus, as used herein, the “treatment” of a subject is intended to embrace both prophylactic and therapeutic treatment, and can be used both to limit or to eliminate altogether the symptoms or the occurrence of an infectious disease. The disease event also includes disorders or conditions that may arise from an infectious disease.

The method comprises administering to the subject any of the disclosed compositions in an amount effective to lower the risk of, or to prevent, or to reduce, or to inhibit, or treat an infectious disease. An “effective amount” refers to any amount that achieves a medically desirable result.

The effective amount will vary with the particular condition being treated, the age and physical condition of the subject being treated, the severity of the condition, the duration of the treatment, the nature of the concurrent therapy (if any), the specific route of administration and like factors within the knowledge and expertise of the health practitioner. For example, in connection with an infectious disease, an effective amount is that amount which slows or inhibits the growth of factors associated with the infectious disease.

Likewise, an effective amount for treating an infectious would be an amount sufficient to lessen or inhibit altogether so as to slow or halt the development of or the progression of the infectious disease. It is preferred generally that a maximum dose be used, that is, the highest safe dose according to sound medical judgment.

When used therapeutically, the composition of the invention is administered in therapeutically effective amounts. In general, a “therapeutically effective amount” means that amount necessary to delay the onset of, inhibit the progression of, or halt altogether the particular condition being treated. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount typically varies from 0.01 mg/kg to about 1000 mg/kg of the body weight of the subject. It is expected that doses ranging from 0.1-500 mg/kg, and preferably doses ranging from 0.1-100 mg/kg or 0.1-50 mg/kg will be suitable. In one embodiment, these listed dosages are to be applied per day. In other embodiments, dosages may range from about 0.1 mg/kg to about 200 mg/kg, from about 0.2 mg/kg to about 20 mg/kg, or from about 0.1-2 mg/kg. In other embodiments, the dosages applied can range from about 0.01-1000 mg/kg/day, 0.1-1000 mg/kg/day, 0.1-500 mg/kg/day, 0.1-200 mg/kg/day, 0.1-100 mg/kg/day, 0.1-50 mg/kg/day, 0.1-10 mg/kg/day, 0.1-5 mg/kg/day, 0.1-2 mg/kg/day, 1 μg/kg/day to 10 mg/kg/day, 1-200 μg/kg/day, 1-100 μg/kg/day, 1-50 μg/kg/day, or from 1-25 μg/kg/day. These dosages can be applied in one or more dose administrations daily, for one or more days.

Preferably, such agents are used in a dose, formulation and administration schedule which favor the activity of the agent towards infectious diseases.

The agent of the invention should be administered for a length of time sufficient to provide either or both therapeutic and prophylactic benefit to the subject. Generally, the agent is administered for at least one day. In some instances, particularly where (situation), the agent may be administered for the remainder of the subject's life. The rate at which the agent is administered may vary depending upon the needs of the subject and the mode of administration. For example, it may be necessary in some instances to administer higher and more frequent doses of the agent to a subject for example during or immediately following a disease event, provided still that such doses achieve the medically desirable result. On the other hand, it may be desirable to administer lower doses in order to maintain the medically desirable result once it is achieved. In still other embodiments, the same dose of agent may be administered throughout the treatment period which as described herein may extend throughout the lifetime of the subject. The frequency of administration may vary depending upon the characteristics of the subject. The agent may be administered daily, every 2 days, every 3 days, every 4 days, every 5 days, every week, every 10 days, every 2 weeks, every month, or more, or any time therebetween as if such time was explicitly recited herein.

A variety of administration routes are available. The particular mode selected will depend, of course, upon the particular drug selected, the severity of the condition being treated, and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule.

Such a pharmaceutical composition may include any of the disclosed compositions in combination with any standard physiologically and/or pharmaceutically acceptable carriers which are known in the art. The compositions should be sterile and contain a therapeutically effective amount of the composition in a unit of weight or volume suitable for administration to a patient. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Compositions suitable for parenteral administration conveniently comprise a sterile aqueous preparation of the composition, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulations suitable for oral, subcutaneous, intravenous, intramuscular, etc. administrations can be found in Remington's Pharmaceutical Sciences, Mack Publishing Co., Easton, Pa.

Compositions suitable for oral administration may be presented as discrete units, such as capsules, tablets, lozenges, each containing a predetermined amount of the composition. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of any of the compositions described herein, increasing convenience to the subject and the physician. Many types of release delivery systems are available and known to those of ordinary skill in the art. They include the above-described polymeric systems, as well as polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the composition is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

Any of the disclosed compositions may be administered alone or in combination with the above-described drug therapies by any conventional route, including injection or by gradual infusion over time. The administration may, for example, be oral, intravenous, intraperitoneal, intramuscular, intra-cavity, subcutaneous, or transdermal. When using any of the disclosed compositions, direct administration to the vessel injury site, such as by administration in conjunction with a balloon angioplasty catheter, is preferred.

Listed below are preferred dosage ranges for examples of GSSG compounds and/or salts for subjects having a weight ranging from about 3-150 kg, or from about 40-120 kg.

Preferably, GSSG·inosine and GSSG-inosine-monophosphate (GSSG-IMP), Li₂-GSSG-inosine-monophosphate (Li₂-GSSG-IMP) and/or uracyl-GSSG-inosine are applied for treatment of acute viral hepatitis B and C as well as mixed hepatitis. Preferable dose range is 10-30 mg/day as daily intramuscular or intravenous administration for 20-30 days.

Preferably, GSSG·inosine and GSSG-inosine-monophosphate (GSSG-IMP) and/or GSSG-uracil-monophosphate (GSSG-UMP) are applied for treatment of chronic viral hepatitis B and C. Preferable dose range is 30-60 mg/day as daily intravenous administration for 30 days following with intramuscular administration every other day for 3-6 months.

Preferably, GSSG·inosine as well as GSSG·adenosine, GSSG·uridine, GSSG·thymidine and Na₂-GSSG-thymidine (Na₂-GSSG-TMP) depending on morphologic peculiarities of the liver alterations and severity of functional abnormalities are applied for treatment of cirrhotic stage of chronic hepatitis. Preferable dose range is 30-60 mg/day as daily intravenous administration for 30 days following with intramuscular administration every other day for 3-6 months. Additionally once every 3 months the said agents are introduced through a liver artery dosing 120-20 mg daily for 5-6 days.

Preferably, GSSG·inosine as well as GSSG·cytosine and GSSG-5-methylcytosine are applied for treatment of lung tuberculosis. Preferable dose range is 10-30 mg/day as intravenous or intramuscular administration twice a day for 30 days following with intramuscular or subcutaneous administration every other day for 3-6 months.

Preferably, Na₂-GSSG-guanosine-monophosphate (Na₂-GSSG-GMP) and/or uracil-monophosphate-Li₂-GSSG-guanosine-monophosphate (UMP-Li₂-GSSG-GMP) are applied for treatment of urogenital tuberculosis. Preferable dose range is 60-90 mg/day as daily intravenous or intramuscular administration for 30 days. The repeated therapeutic cycles are administered according to the results of a urine test on presence of mycobacterium tuberculosis.

Preferably, GSSG·inosine, GSSG·dihydrouracil as well as Zn₂-GSSG-TMP, Ag₂-GSSG-UMP and uridine∞GSSG·inosine depending on the disease stage, origin of opportunistic infection and presence of Kaposi's sarcoma are applied for treatment of AIDS as well as cytomegalovirus infection, infections caused by Epstein-Barr virus and/or pneumocysts. Preferable dose range is 30-90 mg/day as daily intravenous or intramuscular administration for 30 days following with intramuscular or subcutaneous administration once every three days for 3 months. The repeated therapeutic cycles are administered according to the results of viral load tests. The given therapeutic regimen is to be applied as a single-agent therapy. In case of combined therapy involving specific antiviral agents the given therapeutic regimen is to be applied for the entire duration of the antiviral chemotherapy.

Preferably, Li₂-GSSG-GMP as well as the D-forms of Na₂-GSSG-cytosine-monophosphate (Na₂-GSSG-CMP) and the D-forms of GSSG·uracil are applied for treatment of herpes. Preferable dose range is 60-90 mg/day as daily intravenous or intramuscular administration for 14 days following with intramuscular or subcutaneous administration of 30-60 mg/day around impaired sites once every other day for the next 14 days.

Preferably, GSSG·uridine, GSSG-4-thio-uracil and Ag₂-GSSG-uracil-monophosphate are applied for treatment of mycoses. Preferable dose range is 60-90 mg/day as daily intravenous or intramuscular administration for 30 days following with intramuscular or subcutaneous administration of 30 mg/day around impaired sites every other day for the next 30 days. Repeated therapeutic cycles are administered according to the laboratory test results.

Preferably, GSSG·inosine, GSSG·adenosine as well as Na₂-GSSG-adenosine-monophosphate (Na₂-GSSG-AMP) are applied for treatment of mycoplasma infections. Preferably, GSSG·inosine, GSSG·guanosine, GSSG·thymine as well as Na₂-GSSG-GMP are applied for treatment of chlamydia infections. Preferable dose range is 30-60 mg/day as daily intravenous or intramuscular administration for the entire duration of antibiotic therapy. Single-agent therapy comprises intramuscular or subcutaneous dosing of 10-30 mg/day twice a week for 30-60 days.

Preferably, GSSG·inosine as well as GSSG·cytosine and GSSG·5-methylcytosine depending on the infecting agent type, for instance, malaria plasmodium or leishmania, are applied for treatment of protozoa infections. Preferable dose range is 10-60 mg/day as intravenous or intramuscular administration twice a day (mornings and evenings) for the entire duration of specific chemotherapy following with intramuscular or subcutaneous administration of 20-30 mg/day once a day for 45 days.

Preferably, the D-forms of GSSG·inosine (D-cysteine) and the D-forms of GSSG·uracil (D-glutamic acid) are applied for treatment of anaerobic infections. Preferable dose range is 90-120 mg/day as intravenous or intramuscular administration twice a day for the entire duration of specific antibiotic therapy following with intramuscular or subcutaneous administration of 30-60 mg/day once a day for 21 days.

Preferably, Li₂-GSSG-GMP, GSSG·inosine as well as the D-forms of GSSG·uracil (D-glutamic acid) are applied for treatment of viral hepatitis A and enteric infections (dysentery, cholera). Preferable dose range is 60-90 mg/day as intravenous or intramuscular administration three times a day for the entire duration of specific chemotherapy following with intramuscular or subcutaneous administration of 10-30 mg/day once a day for 30 days (as a single-agent therapy).

Preferably, GSSG·inosine, GSSG·adenosine, GSSG·uracil and GSSG·thymine depending on type of the flu virus and viruses causing acute respiratory infection (ARI) are applied for treatment of flu and ARI. Preferable dose range is 10-30 mg/day as intramuscular or subcutaneous administration once a day for 7-10 days. In case of flu epidemic a repeated therapeutic cycle of the given agents as a single-agent therapy in 7 days after the first treatment cycle is administered dosing 10 mg/day once a day for 10 days.”

The function and advantage of these and other embodiments of the present invention will be more fully understood from the examples below. The following examples are intended to illustrate the benefits of the present invention, but do not exemplify the full scope of the invention, considering their application on the wide range of possible infectious diseases.

### EXAMPLE

### Synthesis of GSSG·inosine

400 g (1.3 M) of reduced glutathione (GSH) is dissolved in 700 mL of distilled water with stirring and maintained at a temperature of 12-15° C. (ice bath). 163 mL (0.65M) of 4N solution of NaOH is then added. After complete dissolution, 295 mL of 3% solution of H₂O₂ is added slowly while maintaining the solution in an ice bath to preserve the temperature at 12-15° C. The solution is stirred for another 3-4 hours, keeping the solution temperature below 20° C. The completeness of the reaction is monitored by HPLC (GSSG target content 98% or more). The pH of the solution is also monitored, and if necessary, 4N solution of NaOH is added to adjust the pH to 5.5.

174.2 g (0.65 M) of inosine (as homogenous dry powder) is then added and stirring is continued at 23-25° C. for 10-12 hours, ensuring that the inosine is completely dissolved.

The resultant solution is passed through a filter of at least 0.7 micron. The filtered solution should be transparent, colorless and without opalescence, pH 5.3+/−0.2. The pH is adjusted to 6.0 with a 1 N to 4 N solution of NaOH. Peaks assigned to GSSG and inosine (Nucleosil C18,MeCN-0.1% TFA) are checked via HPLC.

The resultant solution is lyophilized according to the following recommendations (see also a lyophilization schedule attached):

Cool the shelves up to −20+/−0.2C;

Load the solution onto the shelves;

Freeze the solution up to −35+/−0.2C;

Hold the frozen material for 3+/−0.5 hours at the above temperature;

Switch off the shelves;

Switch on condensator cooling up to −54+/−2C for 15+/−3 min;

Vacuum for 30+/−5 min to reach a level of 4-6 Pa;

Hold the material for 2 hours;

Warm the shelves gradually to achieve constant temperature of the shelves and material

### Example 1

### Synthesis of inosyl-5′-phosphoryl-GSSG-Na₂

(I.) General Drug Characteristics.

1. Name: 9-β-D-ribofuranosyl-5′-phosphoryl-N-bis-(γ-L-glutamyl)-L-cysteinyl-bis-glycine disodium (dilithium) salt.

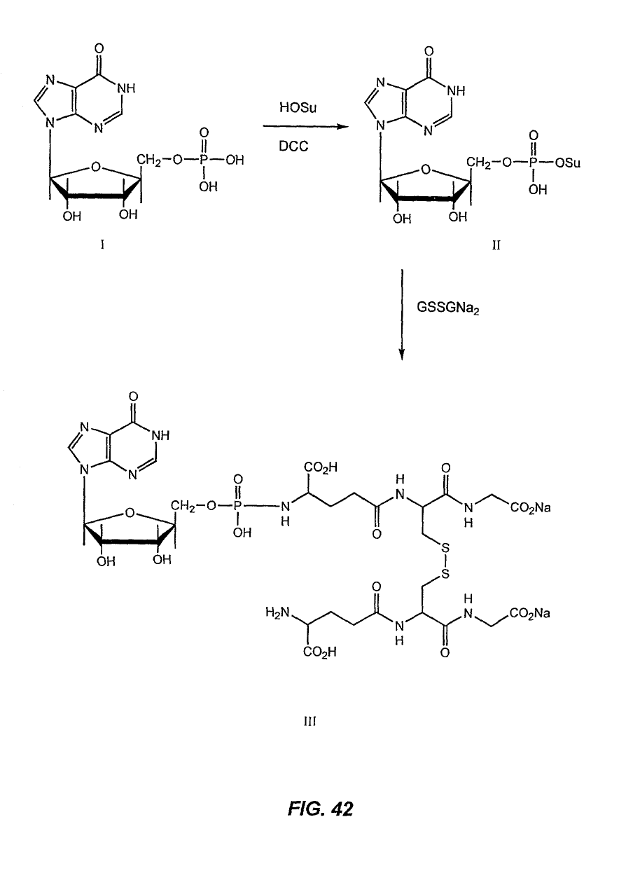

2. Structural formula—see FIG. 42

3. Gross-formula: C₃₆H₅₅N₁₀O₁₉Na₂S₂P

4. Molecular weight: 1072,96 (disodium salt).

5. Appearance: white odorless powder.

6. Solubility: soluble in water, 0.9% isotonic solution of sodium chloride for injections; insoluble in 95% alcohol, chloroform, ether and other organic solvents.

7. Solution transparency and color: 0.05 g of the drug solution in 10 ml of water is transparent and colorless.

8. pH of 0.1% solution: 4.5-5.5 (potentiometry).

9. Authenticity:

a) amino-acid analysis (6 n HCl, 110° C., 20 hrs.), (error margin 20%, for cysteine-35%), in correspondence: glycine-2.00; glutamic acid-2.0; cysteine-2.0.

b) NMR(¹H)-spectroscopy, according to—“BRUKER” AM 500, 500 MHz, D₂O.

| δ | Fragment | Amino-acid |
|---|---|---|
| 4,70 | —C_{α}H— | Cys |
| | \| | |
| 3,75 | —C_{α}H— | Glu |
| | \| | |
| 3,27 | —CH₂— | Gly |
| 2,95 | —CH₂— | Cys |
| 2,52 | —CH₂— | Glu |
| 2,15 | —CH₂— | Glu |

c) HPLC-release time corresponds to the standard.

10. Purity (main substance content):

a) At HPLC: not less than 97%:

Device: BECKMAN “Gold Nouveau Chromatography Data System” Version 1.0, Diode Array Detector Module 126.

Assay—20 μl of 0.1% drug solution, chromatography on the column ULTRASPERE ODS 250×4.6 mm with a converted C₁₈ phase in isocratic mode acetonitrile-0.1% trifluoroacetic acid (2:98); flow rate 1 m/min., detecting at 220 nm, scanning 190-600 nm, PDA functions—Contour Plot, 3D.

b) Amino-acid assay: 85% (the assay as per Item 9a with exact weight);

c) Thin-layer chromatography is homogenous, the test is performed at introduction of 5 μl of the 1% drug solution in the band. Plates—Kieselgel 60_{f} (Merck) 10×5 cm, system: n-butanol—acetic acid—water (4:1:1). Development—as per standard methods—ninhydrine and chlorine/benzidine. R_{f}=0,15;

d) Sodium (Na) content according to the emission spectral method is: 4.8%.

11. Elements detected content, μg/g:

| Silver (Ag) | <1.0 (less than 0.0001%) |
|---|---|
| Aluminum (Al) | 2.0 |
| Arsenic (As) | <1.0 |
| Barium (Ba) | <0.50 |
| Beryllium (Be) | <0.05 |
| Calcium (Ca) | 7.0 |
| Cadmium (Cd) | <0.05 |
| Cobalt (Co) | <0.5 |
| Chromium (Cr) | 1.7 |
| Copper (Cu) | <0.5 |
| Iron (Fe) | <1.0 |
| Potassium (K) | <2.5 |
| Selenium (Se) | <2.0 |
| Magnesium (Mg) | <2.5 |
| Manganese (Mn) | <0.2 |
| Molybdenum (Mo) | <0.2 |
| Nickel (Ni) | <0.5 |
| Lead (Pb) | <0.40 |
| Strontium (Sr) | 1.9 |
| Titanium (Ti) | <0.5 |
| Vanadium (V) | <0.5 |
| Zinc (Zn) | 0.65 |
| Antimony (Sb) | <0.5 |

Determination Method:

The exact assay weight (about 50 mg) is dissolved in 50 ml of double-distilled water and the solution is used for the test.

The platinum content is determined quantifiably by the method of mass spectrometric analysis with inductively bound plasma at a PQe device (VG Elemental, England). The test relative precision is 5%.

Content of other elements is determined quantifiably by atomic-emission spectroscopy with inductive bound plasma on a TRACE 61E (Thermo Jarell Ash, USA). The test relative precision is 5%.

12. Weight loss at drying: 10% at drying till the constant weight at 100° C. in vacuum (1 mm Hg) above CaCl₂ and P₂O₅.

(II.) Synthesis Method Description.

13. Process chemical scheme—see FIG. 42.

I—inosine-5-monophosphate

HOSu—dicyclohexylcarbodiimide

II—oxysuccinimide activated ether of inosine-5-monophosphate

III—inosine-5-monophosphoryl-N-glutathione

14. Method Description

Inosine-5-monophosphate (I) is dissolved in dimethylformamide and along with stirring and cooling to 0-5° C. 1 equivalent of N-oxysuccinimide and 1.2 equivalent of N,N-dicyclohexylcarbodiimide. Then the obtained mixture is stirred cooling for 1 hour, and then at room temperature for 12 hours. Precipitated dicyclocarbamides is filtered and 3 equivalents of disodium salt of oxidized glutathione is added to the residue. The obtained mixture is stirred for 24 hours at room temperature. Then dimethylformamide is evaporated and the product is purified by preparative HPLC as per the above-described mode. Control—by UV spectrum by absorption band of the purine base of 260 nm.

### Example 2

### Antiviral Activity of GSSG·inosine

The Effect of GGSG·inosine on the Course of Rift Valley Fever.

Rift valley fever (RVF) is an acute feverish disease of viral origin affecting domestic animals and humans. The human disease is characterized with acute onset, rapid development of feverish symptoms, pains in joints and extremities, eye affection, hemorrhagic diathesis symptoms. One of the typical RVF signs in humans and animals is viral liver affection causing hemorrhages and massive necrosis of liver parenchymal tissue. The disease is accompanied with leukopenia as well.

Current means of etiotropic and pathogenic therapy for RVF have insufficient activity. Taking into account details of the RVF pathogenesis, particularly, high affinity of the infecting agent to hepatocytes the experimental model of the said infection appeared to be efficient to assess antiviral activity of GSSG·inosine.

1. Study Materials and Methods

1.1. Animals.

In the study there were used adult unbred white male mice weighing 18-21 g obtained from the farm “Rappolovo” of the Russian academy of Medical Sciences.

1.2. Infecting Agent.

For the RVF model there was utilized the viral strain isolated in Uzbekistan in 1987 from the human having the hemorrhagic fever kept in the Viral Culture Museum at the Research Institute of Military Medicine. The brain suspension of new-born mice infected with the said viral strain intracerebrally was used as an inoculate. The virus infecting doses in the present study ranged from 1 to 20 LD₅₀, and the infecting agent was introduced intraperitoneally. The follow-up period lasted for 14 days. 1.3. Tested Articles.

GSSG·inosine—drug form for injections (1% and 3%)—was applied in doses 3, 10 and 30 mg/kg per single injection. The drug was introduced intraperitoneally once a day during 6-7 days. The introduction onset—1-2 days prior to infection, then the drug was introduced in the infecting day (4 hrs. prior to infection) and the introduction continued for 4 days more. Thus, in total, the treatment regimen comprised 6-7 injections of GSSG·inosine.

Ribamidil, the comparison agent (generic of imported agents such as Virazol, Ribavirin), manufactured by Olaine (Latvia), is a drug form for injections. At the moment Ribamidil is one of the most effective antiviral chemotherapeutic agents, particularly, in case of hemorrhagic fevers. The drug was introduced subcutaneously in accordance with optimal, previously developed regimen: a single (daily) dosing was 100 mg/kg, and the introduction terms correspond with the time of GSSG·inosine administration.

1.4. Efficacy Endpoints.

The protective efficacy of the drugs was estimated by the survival rate (%) and median life-span (days) of the experimental and control animals.

2. Study Results

2.1. Effect of a Single-Agent, GSSG·inosine-Based Therapy on RVF Outcome

In the first experiment series GSSG·inosine efficacy was studied for a single-agent therapy therewith. The drug was introduced before infection of the animals with the infecting agent, by 1^{st} regimen 48 hrs. prior to infection, and by two others—4 hrs. prior to infection. Generally the treatment cycles comprised 6-7 intraperitoneal injections of GSSG·inosine in the following single doses—3, 10 and 30 mg/kg. Ribamidil was introduced subcutaneously in the subtherapeutic dose of 100 mg/kg during 5 days.

Incubation in the control group animals lasted for 6 days. However, in the experimental animals the infection signs appeared 2 days later. The final results of the said experiment are given in the Table 1.

Along the data presented the control group morbidity rate infected with the 10-20 LD₅₀ dose amounted 100%. In case of the infecting dose of 1-2 LD₅₀ 67% of the infected mice died. The comparison agent, Ribamidil, taken as a positive control protected from the lethal outcome about one-third of the animals infected with high viral dose and up to 60% of the animals infected with low doses. GSSG·inosine at the given model of the acute generalized infection also exhibited protective effect improving the animal survival rate in 22-59% comparing to the control depending on the therapeutic dose and infection severity.

In the 2^{nd} experimental series protective effectiveness of combined introduction of Ribamidil and GSSG·inosine was assessed. The drugs were applied in the dosages and regimens similar to the 1^{st} series. The said experiment results are presented in the Table 2. The data obtained indicated that the antiviral effect of the combined Ribamidil and GSSG-inosine administration in case of the experimental RVF virus-induced infection is higher than for each agent separately.

At the 3^{rd} experiment series there was studied the GSSG·inosine influence on the RVF course regarding the survival indices and appearance of hemorrhages on the liver surface in the experimental and control mice autopsied during development of the said infection.

For infection the virus was taken in the dose of 4 LD₅₀, and the infection was introduced intraperitoneally.

GSSG·inosine also was introduced intraperitoneally in the two following dosages—3 and 30 mg/kg—during 7 days. The treatment was started immediately after infecting of the animals, and then—every day (once a day). The animals were followed up for 10 days (Table 3).

The given regimen of the GSSG·inosine administration in dose 3 mg/kg was found to possess protective effect regarding the experimental infection in white mice induced by intraperitoneal infection with 4LD₅₀ of the Rift valley fever virus increasing by about two times the animal survival rate (66% vs. 31% in the control). One should note that the general state of the experimental animals was considerably better than the control ones, in particular, the food consumption was much better during the entire monitoring period whilst the control group mice even by the 5^{th} day exhibited anorexia.

The survived mice of the experimental and control groups were autopsied at 5^{th} day (downfall onset) and 10^{th} day (experiment completion). The externally healthy mice were taken for the autopsy. The dead mice were not examined. At the 5^{th} day 1 animal from each group, and at the 10^{th} day—2 animals from the experimental groups and 3 animals from the control one were autopsied. Visual liver inspection indicated that number of the hemorrhagic sites in the experimental animals was less than in the control. In the latter there were counted more than 15-20 sites at each liver while in the experimental animals there were only 1 to 3, moreover, the better index was noted in the animals treated with the higher GSSG·inosine dose, i.e. 30 mg/kg (the sites were not found).

Thus, GSSG·inosine, the new systemic cell-protector and immunomodulator, has antiviral activity and improves resistance of the white mice to the RVF infecting agent that induces lethal generalized infection with liver and other organs affection in the said animals. The intraperitoneal GSSG·inosine introduction with single dosing of 3, 10 or 30 mg/kg for 6 days in case of the experimental RVF in the white mice infected with viral 1-20 LD₅₀ allowed to improve the animal survival rate in 30-60% as well as considerably (by 2 times and more) increase their life-span. Introduction of GSSG·inosine in case of the experimental RVF in the white mice along with the antiviral agent, Ribamidil, improved protective effect of the latter that is indicated with better survival rate (in 20%) and life-span (by 2 times) of the infected mice.

The Effect of GSSG·inosine on the Course of Generalized Herpetic Infection in White Mice.

In the study white unbred male mice weighing 12-14 g obtained from the Rappolovo farm of the Russian Academy of Medical Sciences were used.

For the herpetic infection model there was used the herpes simplex virus, strain L-2, from the museum at the Research Institute of Military Medicine. The animals were infected intraperitoneally with inoculate obtained from brain emulsion of infected and diseased new-born mice.

GSSG·inosine was introduced every day, once a day in the daily dose 30 mg/kg during 5 days; the introduction was started 2 hrs prior to infection.

The herpetic infection proceeds along with immunodeficiency induced with Cyclophosphamide (CPA), group 1; Hydrocortisone (HC)—group 2; radiation (Rad)—group 3.

The comparison agent—Cyclopherone (once a day, 2 hrs prior to infecting in dose 100 mg/kg).

Follow-up—14 days.

Results

The experiment performed exhibited that GSSG·inosine has protective effect regarding DNA viruses that the herpes simplex virus relates to. Even in conditions of lethal infecting dosing GSSG·inosine facilitated survival up to about 30% of the animals while all control animals died (Table 4).

The Effect of GSSG·inosine on the Course of Infection Caused by Horse VEnezuela Encephalomyelitis (HVE) Virus in Animals.

In the study unbred white male mice weighing 18-20 g obtained from the Rappolovo farm of the Russian Academy of Medical Sciences were used, 144 animals in total.

The infecting agent is the HVE virus, strain “TRINIDAD”; infecting doses—1 and 2 LD₅₀ The virus was introduced subcutaneously.

The Tested Articles: GSSG·inosine. The drugs were introduced intraperitoneally dosing 3 and 30 mg/kg; dry samples were dissolved in normal saline to obtain an appropriate single dose in volume of 0.5 ml.

The drug dosing: the drugs were introduced in the given doses as per 2 regimens—preventive (−72 hrs, −48 hrs, −24 hrs, 0) and emergency-preventive (+2, +24, +48, +72, +96, +120 hrs).

The comparison agent (positive control): interferon inducer CYCLOPHERONE (CP) in dose 50 mg/kg introduced 4 hrs prior to infection.

Viral control: the animals were infected with virus-containing material and no treatment was applied.

Combined drug administration: for 2 experimental groups combined preventive administration of GSSG·inosine in dose 3 mg/kg and Cyclopherone in dose 50 mg/kg was applied.

The animals were followed up for 14 days after infecting.

The drug efficacy endpoints: through differences of the survival rate indices (%) and median life-span (T, days) of the experimental and control animals.

The results obtained are presented in the Table 5.

As it follows from the data obtained the infecting VHE virus dose in the given study was found to be even to some extent less than the estimated one—death after the 2 LD₅₀ of the infecting agent occurred in 50% of the control animal group, and after the 1 LD₅₀ one—in 33%.

In the groups No. 1, 4 and 5 at the 3 ^{rd} and 4^{th} days after the infection there was noted non-specific death of 1-2 animals that, usually, is caused by either toxic agents or trauma. The specific VHE-induced mortality in white mice is usually registered from the 5^{th} day after the infection.

The comparison agent, CYCLOPHERONE, applied in the subtherapeutic dose of 50 mg/kg did not exhibit preventive activity after the 2LD₅₀ introduction, and in case of the 1 LD₅₀ introduction the preventive effect equaled to 33%.

GSSG·inosine dosed as 30 mg/kg exhibited antiviral activity. In case of preventive regimen the preventive activity thereof equaled to the Cyclopherone efficacy (improved survival rate in 33%, significant increase of the life-span).

In the experiments with combined prevention one could note clear positive activity of GSSG·inosine regarding both the survival rate index (increased in 17-30%) and values of the T index, i.e. median life-span (improved by 3 times).

Antiviral (Anti-Influenza) Activity of GSSG·inosine.

In the chemotherapy laboratory of the Influenza Institute of the Russian Federation Ministry of Health (Saint-Petersburg) the GSSG·inosine antiviral activity regarding the influenza A virus (H3 No. 2) was studied. The antiviral activity was determined by capacity of the tested Article to depress the influenza virus reproduction on model of surviving fragments of chorion-allantois membrane of chicken embryo (CAM).

The drugs that decreased titre in the experiment comparing to the control (neutralization index) more than on 2.0 lg LD₅₀, were considered to be active; 1.0 to 2.0 lg LD₅₀—moderately active and below 1.0 lg LD₅₀—inactive.

Preliminary studies of the GSSG·inosine toxicity exhibited that even in concentrations 1 mg/0.5 ml they did not cause damaging impact on CAM cells. Thus, the maximal tolerating dose (MTD) was >1000 μg/0.5 ml.

The study results on antiviral activity of GSSG·inosine samples regarding the model influenza virus A/Hong-Kong/1/68(NZ No. 2) are presented in the Table 6.

As one can see from the presented data the GSSG·inosine samples exhibited antiviral activity regarding the influenza A virus.

Thus, the data presented confirmed choice for experimental models of toxic hepatitis to study the GSSG·inosine specific pharmacological activity followed with extrapolation of the results also on the viral liver affections.

### Conclusions

1. GSSG·inosine, new systemic cell-protector and immunomodulator, improves resistance of the white mice to the RVF infecting agent that causes in the said animals lethal generalized infection with liver and other organs affection.

2. The intraperitoneal GSSG·inosine introduction in single doses such as 3, 10, 30 mg/kg for 6 days in case of the experimental RFV in the white mice infected with 1-20 LD₅₀ of the virus increased the survival rate in 30-60% as well as to extend significantly (by 2 times and more) their life-span.

3. The GSSG·inosine application in the white mice having the RVF along with the antiviral agent, Ribamidil, allowed to increase the protecting effect of the latter that was indicated with improved survival rate (in 20%) and life-span (by 2 times) of the infected mice.

4. Dosing 30 mg/kg of GSSG·inosine that was introduced for 5 days increased resistance of the immunocompromised white mice to the DNA-containing virus, i.e. herpes simplex.

5. On the neuroviral infection model caused by the VHE infecting agent the antiviral effect of GSSG·inosine was noted. The preventive regimen with GSSG·inosine was found to be more effective than the emergency-preventive one.

6. The antiviral activity study regarding to the influenza A virus exhibited that the GSSG·inosine samples possessed significant antiviral activity (CTI=5) and diminished the viral infectious activity.

Thus, the unique property of GSSG·inosine to induce apoptotic mechanisms in the virus-infected cells and to activate proliferation and differentiation in the normal cells provides high efficacy of GSSG·inosine regarding to wide range of viral infections.

### Example 3

### Enhanced Apoptosis Inducer Expression—Fas/APOI—Receptor (CD95) in the Virus-Infected Hepatocytes by GSSG·inosine

Fas-receptors called also as antigen CD95+ are initial elements of the receptor-mediated apoptotic cascade. The liver tissue is rich in these receptors and therefore the hepatocyte apoptosis is usually proceeded through Fas-dependent mechanisms. Activation of the Fas-receptors in the defective hepatocytes causes death of genetically altered or virus-infected cells. Benign course of disease caused by hepatitis C virus was shown to be associated with accumulation of Fas-receptors in the virus-infected cells that facilitated their programmed death and, thereupon, elimination of the infected cells. Presence in the extracellular part of the Fas-receptors domains rich in reactive cysteines predetermines its activation in case of sharp changes of SH-group state in the intercellular medium. It could occur after changing of the oxidized and reduced glutathione ratio at treatment of the patients with the Glutoxim family agents. At the present study we explored influence of treatment with GSSG·inosine on the Fas-receptor expression in liver biopsy samples of such patients.

The patient groups for the examination with liver biopsy were formed depending on clinical course of the disease, virus type and administration of GSSG·inosine instead of conventional therapeutics. The biopsy was taken only from the patients with chronic viral hepatitis B or C. The first biopsy was made to assess impairment of the liver tissue before the treatment. The repeated biopsy was made in 3 (hepatitis B) or 6 months (hepatitis C) after the onset of the conventional therapy or treatment cycles with the GSSG·inosine injections.

Totally 84 hepatitis B patients and 63 hepatitis C patients were examined. The biopsy samples were distributed as per morphologic Knodel's gradation. Thus, in the protocol there were enrolled the patients with moderate liver inflammatory process and with moderate signs of fibrosis substitution of the liver parenchyma. At the beginning of the treatment cycle the PCR detected 500000 to 1000000 copies/ml of viral particles. Eventually, there were included 78 hepatitis B patients and 54 hepatitis C ones.

Beside the morphologic assessment of fibrosis process and detection of viral particles the immunomorphological analysis of the cells containing Fas-receptors was made and also the immunochemical test was used to quantitatively determine amount of Fas-receptors in freshly made sample homogenate. Reagents and antibodies manufactured by DAKO (Denmark) were used for the immunomorphology and the immune analysis was made using kit produced by Oncogene (USA).

The fixed tissue samples from the paraffin blocks were prepared as 5-micron sections. Paraffin was removed and then these sections were are dehydrated as described in the DAKO protocol. The monoclonal antibodies to Fas/Apo1-receptors can recognize it on the surface. The antibodies were dissolved according to instruction (1:150) and placed on the slide with the sample. Then it was incubated in a wet camera for 30 minutes or 1 hour according to the enclosed scheme. The unbound antibodies were rinsed twice in 50 ml of PBS-buffer for 2 minutes and then bound primary antibodies were exhibited by secondary antibodies containing 4 biotin mark using the DAKO kit (DAKO LSAB kit k675). The bound biotin was exhibited by streptavidin-peroxidase conjugate in presence of chromogenic substrate, for instance, diaminobenzidine (DAB) as described in the protocol. The corresponding components and reagents are enclosed in the DAKO kit. Assessment of the ration of the cells with exhibited Fas-receptors to other cells was made according to the protocol.

The Fas-antigen immune assay was made with the cell lysate of the fresh biopsy. The enzyme immune assay was made by the “sandwich” method using mice monoclonal antibodies to human Fas-protein immobilized in the wells of the Oncogene 96-well plate (USA). The lysate hourly incubation causes binding of the Fas-antigen and at following rinse the bound Fas-antigen stays in the wells. In the rinsed wells other specific “biotinized” antibodies to Fas-antigen were added and after they bound to the Fas-antigen the wells were rinsed again. Then the conjugate of streptavidin with horseradish peroxidase was added. Streptavidin connected to horseradish peroxidase specifically links to biotin of the secondary antibodies, and after the rinse the entire complex stayed in the wells. Then the chromogenic substrate of tetramethyl benzidine (TMB) was introduced into the wells that being colorless substance under action of the peroxidase transforms into colored bright blue product that can be registered by spectrophotometry. The ready Fas-APO-1 kits of Oncogene™ Research Products (USA).

To the piece of the biopsy a lysating buffer was added with the 10:1 ratio and it was homogenized. Then it was incubated for 30 minutes on ice with periodical stirring. The cell residues were removed by centrifuging for 5 minutes at 12.000 rpm in an Eppendorf centrifuge. The supernatant is used immediately or kept at −80° C.

100 μl of the cell lysate supernatant were introduced in the wells of the well-plate with immobilized antibodies and incubated for 1 hour at room temperature. Then it was thrice (3×) rinsed by the rinsing buffer. Then 100 μl of the detecting antibodies were added into each well and incubated for another hour at room temperature. Then it was thrice (3×) rinsed by the rinsing buffer. Then the streptavidin-peroxidase conjugate was dissolved as 1:400 and it was added by 100 μl into each well. Then it was incubated for another 30 minutes at room temperature. Then the wells were rinsed by the rinsing buffer. Then 100 μl of the chromogenic substrate solution were added for 30 minutes. Then 100 μl of the stop-solution were added (2.5 N sulfuric acid) and the solution absorption was measured in each well at the spectrophotometer or immune assay reader with the wave-length 450/540 nm. The measurement was made not later than 30 minutes after the stop-solution introduction. The results were compared with the control samples enclosed with the kit. The large percentage of the cells of both the hepatitis B and hepatitis C patients were shown to be enriched with the Fas-receptors (Tables 7, 8).

Determination of the Fas-receptor content in the fresh homogenate of part of the biopsies also indicated accumulation of the Fas-receptors in the liver tissue after the GSSG-inosine treatment in case of both hepatitis B and especially hepatitis C (Table 9). For instance, in the supernatant of the hepatocyte lysate-homogenate obtained from the fresh biopsies the Fas content was less than 2 U/mg comparing to the Fas-standards.

The viral infection presence caused significant increase of the Fas-receptor content. At repeated test in 3 (hepatitis B) or 6 (hepatitis C) months in the patients receiving conventional therapy the Fas-receptor content was the same. Contrariwise, in the patients treated with the GSSG-inosine the Fas-receptor content increased significantly.

Discussion.

The Fas-dependent apoptosis is the main pathway of the hepatocyte programmed cell death. Presence of domains rich on reactive cysteines in the Fas-receptor extracellular part suggested that GSSG·inosine facilitates aggregation and, therefore, activation of the Fas-receptors due to wave-like changing of the oxidized and reduced SH-group content ratio in the extracellular glutathione pool. Moreover, the intracellular cascade of caspases performing the Fas-dependent cell death signals being the cysteine proteases also can be activated by GSSG·inosine.

### Conclusion

The Fas-dependent apoptosis activation facilitates elimination of the virus-infected cells. However, due to virus-dependent synthesis of the apoptotic inhibitors the Fas-induced processes cannot be fully performed and then the virus-infected cells escape apoptosis. Increase of the Fas-receptor content and activity by GSSG·inosine provided more complete elimination of the virus-infected cells. These results correspond to the data on decrease or absence of the hepatitis C virus in the blood flow after the GSSG·inosine treatment.

### Example 4

### Study of ATP-ase/Helicase Activity Inhibition of Hepatitis C Virus by GSSG·inosine, GSSG-IMP and GSSG-UMP in Normal and Virus-Infected Cell Cultures

Capacities of compounds of oxidized glutathione with inosine (GSSG-1), inosine-monophosphate (GSSG-IMP) and uridine-monophosphate (GSSG-UMP) to affect ATP-ase/helicase activity of normal and virus-infected (HEP-2) cells. The said substances were incubated with nuclear lysis products of normal lymphocytes obtained from peripheral blood of healthy donors or with homogenate of HEP-2 cells infected with hepatitis C virus.

The venous blood of healthy volunteers was collected into heparinized test-tubes tested in absence of endotoxin. The mononuclear fraction was obtained by centrifuging in ficoll-pack density gradient (Pharmacia). The cell concentration was lifted to 2×10⁶ cells per 1 ml of complete cultural medium (RPMI 1640) comprising 20 mM HEPES, 2 mM of glutamine, 50 μg/ml of gentamycin and 10% fetal embryonic serum. Cell vitality rate was assessed through test with trypanic blue. Then the cell suspension was again centrifuged to remove remaining ficoll with medium. The sediment was suspended in 1 ml of normal saline and lysated with Nonidet 40 to remove cell nuclei as described by Maniatis et al. Then nuclei were lysated and in the lysis products changes of ATP-ase/helicase activity in presence of tested articles were assessed.

To 0.2 ml of nuclear lysate 0.1 ml of DNA of single-chain phage M13 mp10 annealed with P³²-marked complementary oligonucleotide (length—42 nucleotides) was added. Preliminary there were determined phage DNA concentrations that made possible at autoradiography to discern both minimal untwisting and separation of marked oligonucleotide from the phage DNA and virtually complete separation of the oligonucleotide from the DNA of the single-chain form of the phage M13.

The incubation mixture was also introduced with 0.1 ml of Tris-HCl buffer solution comprising ATP and Mg²⁺. Then 0.1 ml of normal saline or 0.1 of normal saline with addition of the tested article were introduced up to final concentration of 10, 50 or 100 μg/ml.

After 30-minute incubation 10 μl of the sample were processed through vertical electrophoresis in 8% polyacrylamide gel. A high-molecular fraction that is a hybrid of P³²-marked oligonucleotide connected to phage DNA, and a low-molecular fraction, that is a oligonucleotide derived under action of a helicase were obtained. Densitometry of the autoradiographs showed changes of the ratio of the marked oligonucleotide and the P³²-marked one released under action of the helicase.

Assessing helicase activity in the lysate of the culture infected by the hepatitis C virus similar technique was applied, however, instead of the nuclei lysate the cell lysate was used to assess contribution of the virus-induced cytoplasm proteins.

The study results are given in the Tables 10 and 11. As one can see from the Table 1 none of the studied substances in concentration 10 μg/ml does not cause the helicase activity in the nuclei lysate of the donor lymphocytes whereas in concentrations 50 and 100 μg/ml all three studied substances significantly suppress the helicase activity. GSSG-UMP inhibits ATP-ase/helicase activity most of all.

In the virus-infected cell lysate the helicase activity is higher because untwisting of 90% of two-chain DNA hybrids of the single-chain domain M13 with the oligonucleotide appeared at the hybrid concentration of 20 pg/ml vs. 5 pg/ml in the nuclei lysate of the donor lymphocytes. Effect of the studied compounds exhibited also at these higher rates of the helicase activity. Significant inhibition of the helicase activity was noted both at 50 and 100 pg/ml, however, at the latter the ATP-ase/helicase activity inhibition was more pronounced. Introduction of GSSG-UMP provided maximal inhibitory effect on the ATP-ase/helicase activity.

Discussion and Conclusion

The high helicase activity in the cells infected with the hepatitis C virus vs. the donor lymphocytes is likely to be associated with not only proliferative activity but also with expression of the 3^{rd} non-structural protein of the hepatitis C that is an enzyme with the ATP-ase/helicase activity. Suppression of the helicase activity not only in the nuclei of the donor lymphocytes but also in the virus-infected cell lysate suggested that the oxidized glutathione compounds with nucleosides (GSSG-I) or nucleotides (GSSG-IMP, GSSG-UMP) brought forth suppression of the helicase activity of the non-structural protein NS3 of the hepatitis C virus that hindered synthesis of the virus-specific RNA in the infected cells.

### Example 5

### Treatment of Acute Viral Hepatitis B with Prolonged Course by GSSG·inosine

Examples 5-16 involve treatment to human subjects weighing from 40 to 120 kg.

| | |
|---|---|
| Patient: | K. V. V. |
| Gender: | male |
| Age: | 32 |
| Case-history | No. 661 |
| Diagnosis: | Acute viral hepatitis B, replication phase (PCR |
| | HBV+), prolonged course, moderate activity rate |
| Complaints on | General weakness, heaviness in the right under the |
| examination: | ribs, nausea, sweating |
| Past history: | The disease started in 01.10.98 when sharp pains |
| | in small hand joints appeared followed with pains, |
| | hyperemia and edema in all joints. From 09.10.98 |
| | he noted darkened urine. The patient was admitted |
| | into the viral infectious hospital. After the |
| | treatment cycle (see below) high cytolytic |
| | syndrome was still present. |
| Previous treat- | The detoxicating, spasmolytic, antibacterial |
| ment: | (Canamycin 0.5 twice a day, IM, N 7), anti- |
| | inflammatory (Indomethacin 1 tab. three times a |
| | day) therapy was administered. |
| Treatment regi- | From 29.10.98 till 21.11.98 |
| men with GSSG · | 0- day |
| inosine | IM GSSG · inosine, 1% - 1 ml |
| | 1-14 days |
| | IV GSSG · inosine, 1% - 1 ml, every day |
| | 15-17-19 days |
| | IV GSSG · inosine, 3% - 1 ml |
| | 20-24 days |
| | IM GSSG · inosine, 1% - 1 ml |

State of Patient upon Completion of Treatment.

Significant improvement was noted exhibited with absence of general weakness, nausea and heaviness in the right subcostal area. Laboratory tests—see Tables 12, 13 and 14. Results of an efficacy study of GSSG·inosine for acute viral hepatitis B (nosology similar to the one presented in the Example) obtained from 39 patients are given in Tables 43-46.

### Conclusion

Treatment course with GSSG·isnosine provided positive development of the following key patient's state's indices:

Restoration of biochemical indices

Ceased HBV replication

Absence of Hbs Ag persistence

Improved general state

The follow-up (in 1 and 3 months after the treatment completion) exhibited stability of the said indices and suggested to call this state as early convalescence.

### Example 6

### Treatment of Acute Viral Hepatitis B with Severe Course by GSSG·inosine

| | |
|---|---|
| Patient: | A. J. V. |
| Gender: | female |
| Age: | 18 |
| Case-history | No. 6006 |
| Diagnosis: | Acute viral hepatitis B, replication phase (PCR |
| | HBV+), severe course |
| Complaints on | General weakness, heaviness in the right subcostal |
| examination: | area, nausea, sweating, easy fatigability |
| Past history: | The disease started in 12.03.99 when sharp pains |
| | in small hand joints appeared followed with pains, |
| | hyperemia and edema in all joints. From 16.09.99 |
| | she noted darkened urine. The patient was |
| | admitted into the 26 department of the hospital |
| | named after S. P. Botkin. After the treatment cycle |
| | (see below) high cytolytic syndrome was still |
| | present. |
| Previous treat- | Massive detoxicating, spasmolytic, antibacterial |
| ment: | (Gentamycin 40 mg, twice a day, IM, N 7), anti- |
| | inflammatory (Indomethacine, 1 tab., 3 times a |
| | day) therapy was administered. |
| Treatment regi- | From 08.04.99 till 02.05.99 |
| men with GSSG · | 0- day |
| inosine | IM GSSG · inosine 1% - 1 ml |
| | 1-14 days |
| | IV GSSG · inosine 1% - 1 ml |
| | 15-17-19 days |
| | IV GSSG · inosine 3% - 1 ml |
| | 20-24 days |
| | IM GSSG · inosine 1% - 1 ml |

State of Patient upon Completion of Treatment.

Significant improvement was noted exhibited with absence of general weakness, nausea and heaviness in the right subcostal area. Laboratory tests—see Tables 15, 16 and 17. Results of an efficacy study of GSSG·inosine for acute viral hepatitis B with severe course obtained from 35 patients are give in Tables 50-53.

### Conclusion

Treatment course with GSSG·inosine provided positive development of the following patient's state's objective indices:

Restoration to norm of biochemical indices

Ceased HBV replication

Absence of Hbs Ag persistence

Improved general state

The follow-up (in 1 month after the treatment completion) exhibited stability of the said indices and suggested to call this state as early convalescence.

### Example 7

### Treatment of Chronic Viral Hepatitis B by GSSG·inosine

| | |
|---|---|
| Patient: | K. M. D. |
| Gender | female |
| Age: | 41 |
| Diagnosis: | Chronic viral hepatitis B (HbsAg+), replication phase |
| | (PCR HBV+), moderate activity rate. Concomitant |
| | diseases: chronic cholecystitis, chronic pancreatitis. |
| | Obesity (Grade II). |
| Liver biopsy | Beam and lobular liver structure is retained. The portal |
| (baseline) | tract is dilated due to growth of the connective tissue |
| | forming portoportal septa. In the periportal connective |
| | tissue there is moderate lympho-macrophagal infiltra- |
| | tion (3 points). The inner border lamina is partially |
| | destroyed, there are stepped necroses (2 points). In the |
| | lobules there are focal necroses (1 point). There is |
| | intralobular and pericentral lymphoid infiltration, |
| | vacuolization of the hepatocyte cytoplasm along with |
| | degeneration and polymorph nuclei; dim-hyaloid |
| | hepatocytes. Conclusion: chronic hepatitis with mild |
| | activity (Knodel's histologic activity index = 6) and |
| | moderate fibrosis. |
| Liver biopsy | Beam and lobular liver structure is retained. The portal |
| (12 months after | tract is slightly dilated due to growth of the connective |
| the treatment | tissue. In the periportal connective tissue there is mild |
| completion) | lympho-macrophagal infiltration (1 point). The inner |
| | border lamina is retained. There is inconsiderable |
| | intralobular and pericentral lymphoid infiltration. |
| | Conclusion: chronic hepatitis with minimal activity |
| | (Knodel's histologic activity index = 1) and mild |
| | fibrosis. |
| Complaints on | General weakness, no possibility to perform usual |
| examination: | activities. |
| Past history: | The patient was at the hospital from 9 till 30.04.97 |
| | with diagnosed chronic viral hepatitis B. The disease |
| | was accompanied with moderate cytolytic syndrome. |
| | The detoxicating therapy was applied. During the |
| | treatment the ALT rate decreased from 15.5 to |
| | 7.7 mmol/hr. 1., and on discharge date bilirubin |
| | content was normal. |
| Previous treat- | Due to viral replicative activity (PCR HBV+) the |
| ment: | Acyclovir course was administered from 25.04.97 for |
| | 21 days, followed with the Cycloferon course from |
| | 26.05.97. During the treatment the Alt rate varied |
| | from 7.4 to 3.4 mmol/hr. 1. The viral activity was |
| | suspended for 1 month, and then it reappeared from |
| | 15.10.97. |
| Treatment regi- | From 15.10.97 |
| men with GSSG · | 0- day |
| inosine | IM GSSG · inosine, 1% - 1 ml |
| | 1-14 days |
| | IV GSSG · inosine, 1% - 1 ml |
| | 15-30 days |
| | IV GSSG · inosine, 3% - 1 ml |
| | 31-90 days |
| | IM GSSG · inosine, 3% - 1 ml 3 times a week |

State of Patient upon Completion of Treatment.

For the first time during last 7 months the biochemical indices restored to normal values (ALT—33 U/l, bilirubin—9.0 μmole/l). The patient is in good state, there is no weakness and she can normally tolerate usual physical activity. Laboratory tests—see Tables 18, 19 and 20 and FIG. 30. Results of an efficacy study of GSSG·inosine for chronic viral hepatitis B obtained from 62 patients are given in Tables 47-49.

### Conclusion

Treatment with GSSG·inosine provided the following:

Ceased HBV replication

Normalization of biochemical indices

Normalization of immune indices and cytokine status

Considerable improvement of morphologic indices (decreased Knodel's index and fibrosis)

Thus, GSSG-inosine is an effective agent for chronic viral hepatitis B.

### Example 8

### Treatment of Chronic Viral Hepatitis B, Cirrhotic Stage, by GSSG·inosine

| | |
|---|---|
| Patient: | A. V. I. |
| Gender: | male |
| Age: | 59 |
| Diagnosis: | Chronic viral hepatitis B, cirrhotic stage (PCR HBV+), |
| | ascites, portal hypertension |
| Complaints on | General weakness, constant ascites, dizziness |
| Examination: | |
| Past history: | For the first time the patient was in the hospital with |
| | diagnosis “Chronic HBV, cirrhotic stage, ascites” in |
| | 1995. Later the patient was again treated in |
| | April 1997. The present worsening started in October: |
| | increased dyspnea and pulling pains in the left under |
| | the ribs and epigastrium. The patient takes Furosemid |
| | (Lasix) 1 tablet twice a week. On admission: medium |
| | severe state, pallid skin, bright and moist tongue, |
| | heart rate - 110 bpm, blood pressure - 140/90, |
| | respiratory rate - 20/min. At the left lower the |
| | scapular angle there is blunted percussion sound |
| | without breath conduction. The stomach is enlarged |
| | (up to 113 cm in diameter) due to ascites. There is |
| | no shin edema. The patient was treated with detoxi- |
| | cating, symptomatic and antibiotic therapy. Also |
| | infusion therapy with diuretics and proteins was used. |
| Treatment cycle | 1 cycle from 27.11.97 to 20.12.97 1%, IM, 3 times a |
| with GSSG · | week |
| inosine | 2 cycle from 12.01.98 to 06.02.98 1%, IM, 3 times a |
| | week |
| | 3 cycle from 11.02.98 to 14.05.98 1%, IM, 3 times a |
| | week |
| | 4 cycle from 06.06.98 to 10.07.98 1%, IM, 3 times a |
| | week |

State of Patient After the 1^{st} Cycle of Completion.

The patient's state is satisfactory. He noted considerable improvement, active and can walk. The ascites diminished (stomach diameter 89 cm), adequate diuresis and no pains in the left subcostal area. The clinical and laboratory data are given in the table. The patient insisted to take repeated cycles with GSSG·inosine as per regimen. The cycles of the said therapy allowed to support stable good state as well as stable clinical and laboratory indices (Table 21).

### Conclusion

Treatment with GSSG·inosine provided the following:

Positive correction of lymphocyto- and thrombocytopenia

Cessation of HBV replication

Absence of cytolytic syndrome (normalization of bilirubin content and ALT activity)

Decreased ascites

Better quality of life: improved and stable state during 8 follow-up months with enhanced activity, euphrasy and desire to continue this treatment

### Example 9

### Treatment of Chronic Viral Hepatitis B, Cirrhotic Stage, by GSSG·inosine

| | |
|---|---|
| Patient: | T. I. N. |
| Gender: | male |
| Age: | 48 |
| Diagnosis: | Main: Chronic viral hepatitis B (HbsAg+), integration |
| | phase (PCR HBV−), cirrhotic stage |
| | Concomitant: |
| | State after cholecystectomy |
| Complaints on | General weakness, insomnia |
| Examination: | |
| Past history: | The chronic hepatitis was first diagnosed in 1999 at |
| | examination before cholecystectomy. After the surgery |
| | (cholecystectomy) performed at the beginning of |
| | January 2000 the treatment with GSSG · inosine was |
| | started at April 26, 2000. |
| Treatment regi- | 1% solution, 3 times a week for 12 weeks |
| men with GSSG · | |
| inosine | |
| Ultrasound | Baseline of 18.04.00 No. 1344 The liver has normal |
| diagnostics | size, its structure is homogenous, small-grained, the |
| | distinct vessels can be seen at the peripheral area; |
| | the structure is dense. Portal vein - 13 mm, |
| | v. mesenterica superior 14 mm, common bile duct - |
| | 7 mm, spleen - normal size, hypoechogeneic structure |
| | with smooth and distinct shape. The gall-bladder is |
| | removed. The pancreas: head - 22 mm, body - 16 mm, |
| | tail - 20 mm, smooth shape, homogenous, small- |
| | grained structure, echogeneity similar to the liver. |
| | The kidneys are of normal size, bean-like shape, with- |
| | out concretions. Conclusion: diffuse liver alterations. |

State of Patient upon Completion of Treatment.

The patient state is satisfactory. He noted significant improvement of his state and ability for normal professional activity. The treatment improved clinical and laboratory indices and quality of life (see Tables 22, 23 and 24).

### Conclusion

Treatment with GSSG·inosine provided the following:

Thrombocytopenia correction

Cytolytic syndrome cessation (normalized bilirubin content and ALT activity)

Cell immunity stimulation (increased CD3, CD4, CD16/56 counts)

considerable improvement of portal blood flow according to the dopplerography

### Example 10

### Treatment of the Patient with Chronic Viral Hepatitis C and Chronic Viral Hepatitis B by GSSG·inosine

| | |
|---|---|
| Patient: | M. V. V. |
| Gender: | male |
| Age: | 18 |
| Case-history: | No. 1043 |
| Diagnosis: | Chronic viral hepatitis C, replication phase (PCR |
| | HCV+), moderate activity; Chronic viral |
| | hepatitis B, integration phase (PCR HBV−), |
| | narcotic intoxication, drug addict. |
| Liver biopsy | Beam and lobular liver structure is retained. The |
| (baseline) | portal tract is slightly dilated due to growth of the |
| | connective tissue. In the periportal connective tissue |
| | there is moderate lympho-macrophagal infiltration |
| | (3 points). The inner border lamina is retained. In |
| | some lobules there are Kaunsilmen's bodies |
| | (1 point). There is intralobular and pericentral |
| | lymphoid infiltration, vacuolization of the |
| | hepatocyte cytoplasm and nuclei of some |
| | hepatocytes. Conclusion: chronic hepatitis with |
| | minimal activity (Knodel's histologic activity |
| | index = 4) and mild fibrosis. |
| Liver biopsy | Beam and lobular liver structure is retained. The |
| (12 months after | portal tract are not altered. In the periportal |
| the treatment | connective tissue there is mild lympho-macrophagal |
| completion) | infiltration (1 point). The inner border lamina is |
| | retained. In some lobules there are Kaunsilmen's |
| | bodies (1 point). There is intralobular and |
| | pericentral lymphoid infiltration, vacuolization of |
| | the hepatocyte cytoplasm and nuclei of some |
| | hepatocytes. Conclusion: chronic hepatitis with |
| | minimal activity (Knodel's histologic activity index = |
| | 2). |
| Complaints on | General weakness, strong pains in the left subcostal |
| examination: | area, knee joints, spine and hand joints |
| Past history: | The patient noted pains in the knee joints and the |
| | spine at the beginning of August 1997. The blood |
| | tests exhibited increased bilirubin to 34.0 μmole/ |
| | l and ALT - 2.1 mmol/hr. 1. During the hospital |
| | examination at 15.08.97 anti HCV IgG and |
| | replicative hepatitis C virus activity were found. |
| Life history: | The patient started to take drugs at his 14, and |
| | intravenously - at 17. At the examination date the |
| | patients takes 2 g of heroin daily and suffers |
| | narcotic abstinence. |
| Previous treat- | The patient was not previously treated. |
| ment: | |
| Treatment regi- | From 15.08.97 |
| men with GSSG · | 0- day |
| inosine | IM GSSG · inosine, 3% - 1 ml |
| | 1-14 days |
| | IV GSSG · inosine, 3% - 1 ml |
| | 15-30 days |
| | IV GSSG · inosine, 3% - 1 ml |
| | The treatment was administered for 3 months. |

State of Patient upon Completion of Treatment.

The patient state is satisfactory. He noted considerable diminution of weakness, no pains in the right subcostal area and the joints. Also the patient said that the period of the drug abstinence was almost painless and shorter. Biochemical indices and ceased replicative viral activity were noted (see Tables 25, 26 and 27and FIG. 31).

### Conclusion

Treatment with GSSG·inosine provided positive development of the disease exhibited with normalization of biochemical, serologic indices and ceased HCV replication. The immune and cytokine status indices correlate with control of the infectious process and absence of the viral replication. Studying the patient's lymphocytes by flow cytometry using monoclonal antibodies to FasAg (CD95+) after the treatment increase of the CD95+-cells was found indicating activation of the programmed cell death in the virus-infected cells. The follow-up in 1 and 3 months after the treatment completion showed stability of the said condition.

The concomitant drug abuse and abstinence at the GSSG·inosine application were controlled earlier and the patient less suffered.

Thus, the treatment cycle with GSSG·inosine for chronic hepatitis C with replicative activity and concomitant drug abuse allowed to obtain the following results:

Restoration of biochemical indices

Restoration of hematologic indices

No replicative activity of the hepatitis C virus

Improved morphologic parameters

Restoration of immune indices and cytokine status

Apoptosis induction in virus-infected cells of peripheral blood

Rapid alleviation of the drug abstinence

Stable therapeutic effect

### Example 11

### Treatment of the Patient with Chronic Viral Hepatitis C, Cirrhotic Stage, by GSSG·inosine

| Patient: | G. N. V. |
|---|---|
| Gender: | female |
| Age: | 48 |

Diagnosis: Chronic viral hepatitis C, cirrhosis (Child C), ascetic syndrome, varicous dilation of the esophageal veins and gastric cardia, Stage II.

Complaints prior to the treatment: Weakness, insomnia, arthralgia, skin itching, gingival hemorrhages.

Disease anamnesis: The disease first appeared in 1993 when chronic viral hepatitis C, cirrhotic stage was diagnosed. Ascites increased starting from 1999 until treatment with the GSSG·inosine agents started.

Treatment course with GSSG-inosine: 1% solution 3 times a weeks for 12 weeks.

Patient's state after the treatment course completion:

The patient's state is satisfactory. He noted significant improvement of the state, and he was fully capable. The treatment applied ensured better clinical and laboratory indices and quality of life of the patient (see Tables 28, 29 and 30). Results of an efficacy study of GSSG-inosine for chronic viral hepatitis C (nosology similar to the one presented in the Example) obtained from 74 patients are given in Tables 54-55.

| | Baseline | After the treatment |
|---|---|---|
| Asthenic syndrome | +++ | + |
| arthralgia | +++ | − |
| skin itching | ++ | + |
| hair condition | − | − |
| gingival hemorrhages | ++ | − |

### Conclusion

The treatment course with GSSG·inosine ensured the following:

Minimal manifestation of ascetic syndrome.

Improvement of portal blood flow according to dopplerographic data.

No dopplerographic signs of portal hypertension (no spleen-renal anastomoses).

Normalization to about normal counts of lymphocytes and platelets.

Improved quality of life.

### Example 12

### Therapeutic Effects of GSSG·inosine in AIDS Patient

Patient: M.V.M.

Sex: male.

Age: 50.

Diagnosis: AIDS, C3 (CDC Atlanta gradation).

Concomitant diseases:Kaposi's sarcoma. Tuberculosis of intrasternal lymph nodes, cytomegalovirus infection, mycotic stomatitis, herpes simplex, eczema, neurosyphilis.

HIV was diagnosed in 1994. Positive immunoblotting—p 24, p 17, p 25, p 18, p 55, p 40, p 68, p 58, p 34, gp 120, gp 160.

Initial patient condition: Severe, temperature to 38.5° C. for more than a month, Karnovsky score—50, progressive worsening of immune indices. Sharp leukopenia (1.8×10¹²) and lymphopenia (570) excluded possibility for application of AZT group agents and other antiviral medicines.

Treatment course with GSSG·inosine (D-form): GSSG·inosine—3%, 1 ml, 3 times a week, intramuscularly, for 12 weeks.

Therapeutic effect after a 1-month treatment:

temperature—37.5° C.;

improved quality of life—Karnovsky score 70;

immune indices trended to restore (lymphocyte count—increased from 570 to 832, CD4⁺/CD8⁺ ratio increased from 0.71 to 0.84);

viral load—89000 copies/ml.

Therapeutic effect after a 2-month treatment:

temperature—36.9° C.;

improved quality of life—Karnovsky score 75;

immune indices still trended to restore (lymphocyte count—736, CD4⁺/CD8⁺ ratio—0.87; CD4⁺-199; CD8⁺-228)

viral load—56000 copies/ml.

Therapeutic effect after treatment completion:

temperature—36.6° C.;

improved quality of life—Karnovsky score 90;

immune indices still trended to restore (lymphocyte count—1350, CD4⁺/CD8⁺ ratio—0,87; CD4⁺-527; CD8⁺-608)

viral load—10000 copies/ml.

Laboratory indices—see Tables 31 and 32.

### Example 13

### Therapeutic Effects of Zn₂-GSSG-TMP¹-AIDS

| Patient: | J. O. I. |
|---|---|
| Sex: | female |
| Age: | 40 |

Diagnosis: HIV-infection (AIDS stage); C2 (CDC Atlanta gradation). Chronic B relapsing herpes simplex. Encephalopathy. Chronic viral hepatitis B (HbsAg+), integration stage. ¹Note: Zn₂-GSSG-TMP-zinc salt of GSSG, which is covalently bound with inosine-monophosphate

On admission on 12.03.96 in the Hospital of Infectious Diseases (AIDS department) the patient complained on general weakness, fatigue, sweating, especially at night, weight loss, memory disturbances, tenderness in wrist joints and left popliteal fossa.

Objective examination: The patient's condition is satisfactory. There are weight loss signs, pallid skin integument and oral cavity mucosa. Neck and axillary lymph nodes enlarged up to 1 cm in diameter. Percussion sound above lungs is not muffled. At auscultation—there are coarse breath sounds, dry disseminated rales. The stomach area is soft, a little tender in epigastric area and there is a small consolidation in the right iliac area. Hands are acrocyanic. Lip scars are caused with multiple herpetic eruptions. Karnovsky score—75.

Treatment course with Zn₂-GSSG-TMP: from 26.03.96 GSSG·inosine, 3%-1 ml, 3 times a week, intramuscularly for 8 weeks.

Therapeutic effect after a 1-month treatment with Zn₂-GSSG-TMP: The patient exhibited diminished weakness. and the night sweating disappeared. She gained 1.8 kg. The lymphocyte count 1276, CD4⁺-319, CD8⁺-370. Karnovsky score—80.

Therapeutic effect after completion of treatment with Zn₂-GSSG-TMP: No complaints were present. The patient's condition was satisfactory: no sweating was reported and the patient became more active. Skin integument became less pallid. She gained 2.5 kg.

The lymphocyte count—2295, CD4⁺-574, CD8⁺-597. Karnovsky score—90.

Specific development of immune and blood indices is given in the Tables 33 and 34.

### Conclusion

Treatment with Zn₂-GSSG-TMP as a single-agent therapy provided:

Improved quality of life

Improved immune indices

Stabilization of hematologic indices

Diminished viral load

### Example 14

### Therapeutic Efficacy of GSSG·inosine for Chlamydia Infection

Patient: S-n I.M., age—29.

Diagnosis: Urogenital chlamydiosis with systemic manifestations. Chronic vesiculoprostatistis. Right joint chronic synovitis, chronic blepharoconjunctivitis.

Anamnesis: Chronic prostatitis was diagnosed about 8.5 years ago. The patient was treated 4 times using modern antibiotic agents such as macrolides, fluoroquinolons and Doxycycline. Last two treatment cycles were administered along with immunotherapy (Cyclopheron, Viferon). Last time, in 1999, the patient was treated in Urology Department of Saint-Petersburg Medical University, however, relapse developed in 3 months manifested as chronic prostatitis exacerbation, knee joint synovitis, smarting eyes, photophobia, eye discharge after night sleep. Therefore, the patient was treated by an ophthalmologist (tetracycline and erythromycin ointment), however, without any positive issues. From March 1992 the patient was administered with the following therapeutics: Bicillin, Canamycin, Doxycycline, Tetracycline, Trichopol, Timidazol, Methacycline, Summamed, Cyfran, Zanozin, Abactal, Rulid, Cyclopheron, Viferon.

In September 2000 physicians of the Department on Military Traumatology and Orthopedy of the Military Medical Academy the patient was sent for consulting to the Department on Infectious Diseases of the Military Medical Academy where the patient was thoroughly examined and administered with complex treatment including introduction of GSSG·inosine and antibiotics. Etiotropic therapy was applied after test of antibiotics sensitivity of chlamydia extracted on cell culture obtained from ejaculate and joint synovial fluid.

On admission: the patient's state is satisfactory. Complaints on general fatigue, weakness, prolong (over 1.5 months) subfebrile temperature, periodic pains in perineum and in the right knee region as well as soft tissue edema and skin hyperemia of the knee region.

In the prostate gland secretion—chronic prostatitis signs. The right knee joint puncture—8 ml of turbid synovial fluid were obtained. After the puncture the joint edema diminished slightly.

Direct immunofluorescent assay (DIFA) revealed significant number of reticular and elementary bodies of C. trachomatis in the prostate secretion and synovial fluid. The serum antibody titer to C. trachomatis (by IFA—indirect immunofluorescent assay) amounted 1:128, and in synovial fluid—1:64. The pure infectious agent culture was obtained in the cell culture from ejaculate and joint synovial fluid, and antibiotics sensitivity was determined.

Development of major laboratory indices is given in the Table 35.

### Conclusion

GSSG·inosine was applied as per the following regimen: 1 ml of 3% solution once every other day for 10 days (5 shots) prior to antibiotic treatment and in the similar manner—during it (8 days—Maxavin and 8 days—Vilprafen). It provided stable clinical and bacteriologic effects. Elimination of chlamydias was proved by main parameters immediately after the treatment, and by PCR—in 3 months after the treatment. GSSG·inosine facilitated shortening of terms for restoration of major immune indices (counts of T- and B-lymphocytes, IL-1, IL-2, IL-8, IFN-α and γ).

### Example 15

Therapeutic Efficacy of GSSG·inosine for Mixed-Infections: Herpes and Chlamydia.

Patient: G.M.M.

Diagnosis: Chlamydiosis. Genital herpes.

Concomitant diagnosis: Chronic pyelonephritis.

Anamnesis: chronic pyelonephritis and chlamydial-herpetic infection were diagnosed about 5 years ago. The patient was treated with all known antibiotic agents (fluoroquinolons, macrolides, tetracyclines) including supportive treatment with Roferon and Cyclopheron. Administration of Cyclopheron was complicated with fever (temperature up to 40° C.), alopecia, skin rash. In 1996 the patient was treated in the Urology department of Central Medical and Sanitary Hospital No. 122 due to pyelonephritis exacerbation.

During 5 years of continuous treatment the antibody titer to Chlamydia trachomatis t decreased to 64 points. But in a month after each treatment course it went up to 150.

From December 1993 till August 1997 the patient was administered with the following agents:

Trichopol, Timidazol, Bicillin, Timogen, Summamed, Metacycline, Dalacin C, Marveron, Doxycycline, Cyfran, Zanocin, Macropen, Clotrimazol, Reoferon, Abactal, Rovamycin, Tarivid, Rulid.

In April 1998 the patient came for treatment to the Urology and Andrology Department of Medical Academy of Post-Graduation Education where she was treated with ark new therapeutics, GSSG-uracyl, combined with antibiotic therapy.

State on admission: satisfactory. Complaints on general weakness, periodic pains in knee joints and pelvic area, vaginal discharge as leukorrhea.

Slight leukocyturia. In blood—autoantibodies to renal tissue, titer—1:4 (Complement-fixation test of V.I Ioffe and K.M. Rosental).

Changes of blood indices are given in the Table 36.

Treatment protocol:

2 antibiotic therapy cycles (Doxycycline—0.1 g, twice a day, for 10 days at 3 to 12 treatment days; Clacid—0.25 g, twice a day, for 10 days at 21 to 30 treatment days).

GSSG-uracyl as per regimen (treatment days—1 to 30).

After monthly treatment:

The patient's state is satisfactory. No complaints are presented. Blood and urea indices are normal.

Conclusion:

Administration of GSSG-uracyl provided the following:

Clinical improvement;

No laboratory signs of chlamydial and herpetic infection (bacterial and serologic tests, PCR);

No autoantibodies to renal tissue, restoration to normal of urea indices.

### Example 16

### Effects of Li₂-GSSG-IMP in Pulmonary Tuberculosis Patient

| Patient: | N. F. V. |
|---|---|
| Gender: | male. |
| Age: | 30. |

Diagnosis: Infiltrative tuberculosis of left lung upper lobe, stage of destruction and seeding, bacterial discharge (+).

Concomitant diagnosis: Chronic bronchitis, exacerbation stage.

Anamnesis: Pulmonary tuberculosis was diagnosed in penitentiary in May 1998. The patient was treated from May till August 1998 in the penitentiary hospital without evident effect, and the disease progressed. On discharge in August 1998 the patient was further treated in the tuberculous hospital in Saint-Petersburg. However, no improvement was obtained, and in October 1998 there was still present massive bacterial discharge and lung destruction cavity enlarged. Also mycobacterial resistance to Streptomycin and Isoniazid was found.

State on admission: Moderately severe, evening temperature—37.5° C., night sweating, productive coughing with pus, Karnovsky score—50, progressing diminution of immune indices. Leukocytopenia, lymphocytopenia, monocytosis, stab shift in neutrophils. In sputum—bacterial discharge (+) by microscopy—up to 100 in the field of vision. X-ray picture: in the upper lobe of the left lung—infiltration with destruction cavity, size 4×3 cm, and seeding sites into the left lung lower lobe.

Treatment cycle by Li₂-GSSG-IMP as supportive treatment for antibacterial therapy (Isoniazid, Rifampicin, Pirasinamid, Amicacin). Li₂-GSSG-IMP—3%, 1 ml, intramuscularly, once a day, 5 times a week, course duration—3 months.

Therapeutic effectiveness in a month:

Temperature normalization;

No night sweating;

improved quality of life: Karnovsky score—70;

The patient gained 4 kg;

Improved hematologic indices, trend towards increase of IL-12 content, restoration of the Th₁/Th₂ ratio to norm;

Diminished bacterial discharge (bacterial discharge (+)—2-4 in the vision field by microscopy), sputum became serous.

Therapeutic effectiveness in 2 months:

Temperature—36.7° C.;

improved quality of life: Karnovsky score—75;

trend to restoration of immune indices;

Bacterial discharge ceased (bacterial discharge (−) by microscopy).

Therapeutic effectiveness on treatment completion:

Temperature—36.6° C.;

improved quality of life: Karnovsky score—90;

restoration of immune indices;

Bacterial discharge ceased (bacterial discharge (−) by flotation—three sequential tests);

X-ray: destruction cavity in the left lung upper lobe healed, outcome—linear scar (see FIGS. 31 and 32).

Laboratory indices—see Table 37.

Conclusion:

Administration of Li₂-GSSG-IMP within combined tuberculosis treatment for 3.5 months provided the following:

Bacterial discharge cessation;

Healing of the lung destruction cavity;

Overcoming of mycobacteria drug resistance

The said effects were not obtained for previous 6 months of conventional antituberculous chemotherapy.

### Example 17

### Hepatoprotective Efficacy of GSSG·inosine Experimental Study of GSSG·inosine Hepatoprotective Activity

The GSSG·inosine hepatoprotective activity was studied on available and reproducible toxic hepatitis models because hepatotoxicity mechanisms such as inflammation, cytolysis and cholestasis are well known to be universal and non-specific and not dependent on the agent inducing the liver impairment. For comparison the known hepatoprotectors Essentiale® (Aventis Pharmaceuticals, Bridgewater, N.J.) and Legalon® (Madaus A G, Koln, Germany) were used.

To model toxic hepatitis dichlorethane and acetaminophen were used. Both compounds were introduced in stomach of the male rats with standard weight (160-170 g) through metal atraumatic tube dosing 500 and 400 mg/kg, respectively, once a day during 4 days mixing with olive oil (1:1). Besides, we used combined introduction of dichlorethane and acetaminophen dosing 300 and 250 mg/kg, respectively. In 10 days all experimental animals exhibited toxic hepatitis as per morphologic liver picture. Starting from that day for 10 days the animals were introduced once a day with GSSG·inosine (5 mg/kg, IM) and comparison agents, i.e. Essentiale® (ampoules) (1 mg/kg IV through tail vein) and Legalon® (Silimarin) (100 mg/kg into a stomach).

The treatment efficacy was assessed through clinical condition, weight changes, relative liver weight, transaminase activity, content of bilirubin, phosphatases, ceruloplasmin, total protein, blood lipids, glycogen, glutathione, SH-groups, cytochromes in the liver, loading tests (hexenal test, bromine-sulfoaleine test) and liver histology.

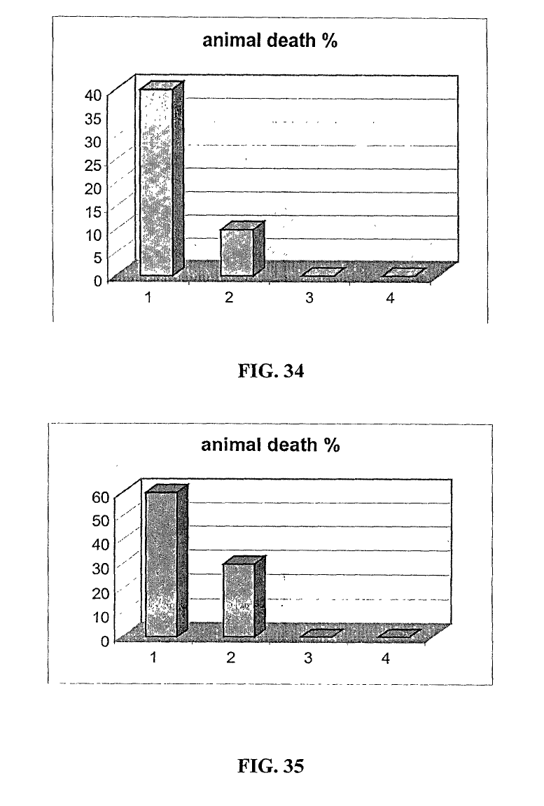

It was found that depending on the toxicant type the animal death at the 20^{th} Day of the Study varied from 40% (in the dichlorethane-poisoned group) to 80% (in the group treated with dichlorethane and acetaminophen) (FIGS. 34, 35 and 36). The most considerable liver impairment was noted after combined introduction of two aforementioned agents that was confirmed with changes of biochemical indices of functional hepatocyte state: increased activity of transaminases, phosphatase, lactate-dehydrogenase, decreased content of total protein, lipids, reserve of blood sulfhydryl group, increased content of bilirubin and ceruloplasmin, significant inhibition of bromine-sulfaleine excretion.

Presence of the toxic hepatitis was confirmed with liver morphology.

The animals died or euthanized in 1 day after peroral dichlorethane introduction exhibited signs of the acute toxic hepatitis. After the microscopy it was found: hepatocyte cytoplasm was densely filled with large fat drops. The liver fat dystrophy was diffuse. Also vacuolization of the hepatocyte cytoplasm and cell body swelling was noted.

Similar changes were found after the acetaminophen treatment. In case of the combined impairment the liver biopsy morphology was more notable and total. Beside the aforementioned changes of the liver parenchyma there were also sites of lobular colliquative necrosis.

Using the combined toxication model the glycogen, reduced glutathione and detoxicating cytochromes content were significantly diminished. The liver impairment was characterized with lowered functional activity expressed by decreased rate of hexenal destruction and bromine-sulfaleine. The GSSG∞inosine application was more effective vs. introduction of Legalon® and Essentiale®.

The GSSG·inosine administration considerably improved the animals' state, prevented their death and had positive influence on morphometric, biochemical and functional indices of the liver tissue.

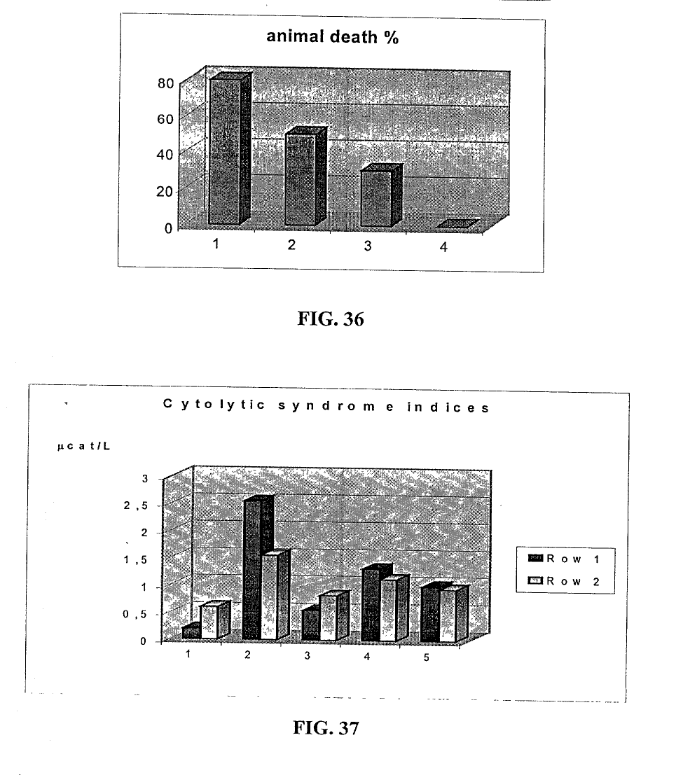

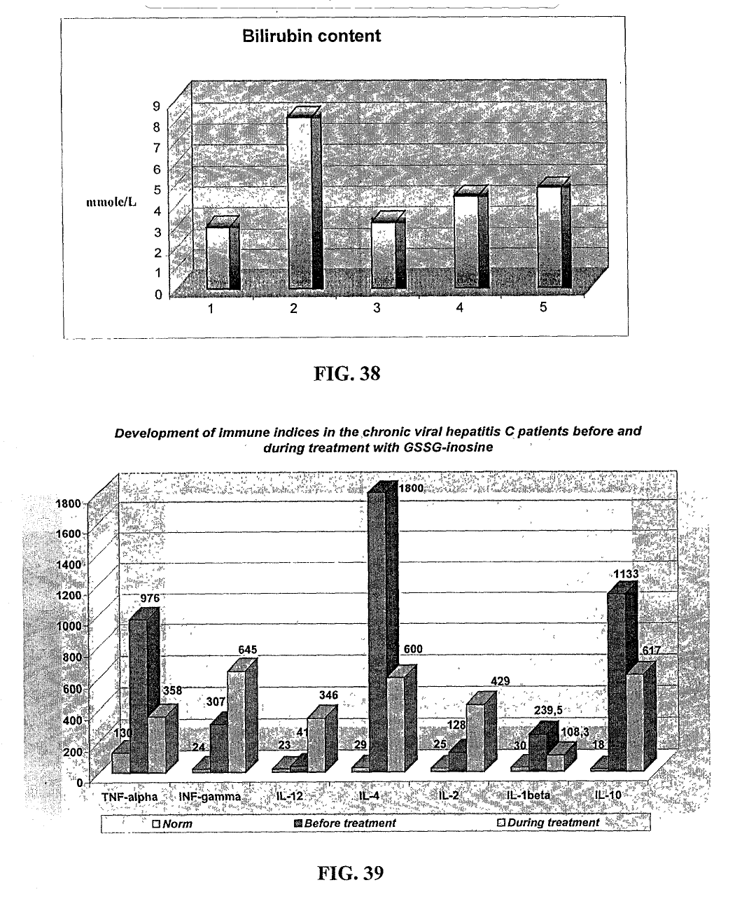

The GSSG·inosine application provided 100% survival rate in the experimental group vs. 80% mortality rate in the control; 45% mortality rate in the Legalon®-treated group and 30% mortality rate in the Essentiale®-treated one (FIG. 36).

Studying the biochemical indices there were evident significant signs of the liver impairment: disturbed protein-synthesizing and lipid-synthesizing functions, activated cytolytic syndrome and cholestasis in conditions of forming of the toxic hepatitis after combined introduction of two aforementioned toxicants, i.e. Acetaminophen and Dichlorethane. Therapy with GSSG·inosine facilitated restoration of main liver functions and main parameters featuring the toxic liver impairment (Tables 38, 39).

The indices that directly characterizes the liver metabolism (glycogen supply, glutathione content, activity of microsome enzymes, relative liver weight) in the animals treated with GSSG·inosine was actually similar to baseline values (Table 40).

Thus, the presented results suggested that GSSG-inosine is an effective hepatoprotective agent. After a 10-day cycle in the rats dosing 5 mg/kg the drug had a considerable therapeutic effect at combined liver impairments induced with Dichlorethane and Acetaminophen restoring negative changes of biochemical status and morphology. The GSSG·inosine therapeutic application provided 100% survival rate in the experimental group vs. 80% mortality rate in the control; 45% mortality rate in the Legalon®-treated group and 30% mortality rate in the Essentiale®-treated one (see Tables 38, 39 and 40).

### Example 18

### Therapeutic Efficacy of GSSG·inosine for Experimental Toxic Liver Cirrhosis

White unbred male rats weighing 180-240 g were used in the study obtained from Rappolovo farm of the Russian Federation Academy of Sciences; in total—60 animals.

The method of Jezequel, A. M. et al. was used to induce liver cirrhosis in the rats. (Dimethylnitrosamine-induced cirrhosis. Evidence for an immunological mechanism. J. Hepatol., 8, 42-52(1989).) As per the said method the animals were introduced intraperitoneally (i/p) introduced for 3 consecutive days with 1% solution of dimethylnitrosoamine (DMNA) dosing about 1 ml/kg, and after the 4-day break the injections were repeated. DMNA was dissolved by normal saline. To obtain considerable cirrhosis 4 such injection cycles were sufficient.

For morphologic examination after narcotized euthanasia samples of liver tissue from central and right side lobes were taken. The liver samples were fixated by mixture of formalin, alcohol and acetic acid. Then paraffin 4-μm-wide sections were made and stained by hematoxylin-eosin or by van-Gison method.

For objectification of influence of the tested articles on connective tissue development in case of experimental DMNA-induced toxic alteration a square occupied by connective tissue was measured at 6 liver sections within 5-7 liver lobules. The van-Gison stained samples where collagen fibers had red color were used for the study. By computer processing the scanned and 200×-magnified image of the liver section was binarized by color amplifying the red-colored fibers. By number of pixels forming the selected objects a relative square occupied by developed collagen (connective tissue) in the liver tissue was calculated.

The measurement results were statistically processed. The difference significance between mean samples using the Student criterion with significance level of p=0.05.

The morphological alterations were assessed on the samples with blind marking reducing risk of aggravation of the study results.

The pathologic process and agent efficacy were assessed by clinical signs (weight, appearance, ascites, motional activity, death) as well as morphologic ones after animal necropsy with following morphologic liver sample examination.

After development of pathologic process, i.e. in 4 weeks after the DMNA introduction onset, all animals were randomized in 4 groups of 12 animals in each. Group No. 1—no treatment, group No. 2—treatment with normal saline, group No. 3—treatment with GSSG·inosine, group No. 4—treatment with comparison agent, Heptral. Group No. 1 included 12 intact animals of the similar population used for morphometry.

Each group was divided in two sub-groups: one was introduced with the tested article for 3 weeks followed with morphologic examination; and the animals of the second sub-group were treated for 6 weeks. The agents were introduced every other day intramuscularly, in femur muscle.

The obtained results indicated the following:

The first experimental group (12 rats) included animals not treated after the DMNA introduction completion. These animals had retarded weight gain (Table 41), for 3-4 weeks they exhibited low motional activity, ate worse; hair were dull and ragged. Morphologic and histologic examination in 3 weeks after the DMNA introduction completion (6 animals) revealed ascites and portal hypertension signs. Examination of the van Gison-stained samples showed the connective tissue amount in the liver septa that resulted in regular structure of the latter. The central vein lumina in the septa nodes were often occluded and substituted with connective tissue. Collateral blood flow went through dilated sinusoids constrained within regular fiber septa frame. Liver cells exhibited dystrophic alterations. The hematoxylin-eosin-stained samples revealed a great deal of cells with sings of marked protein-hydropic dystrophy. Hepatocyte nuclei were in some cases pycnotic and in other—lysated.

Comparing with the aforementioned abnormalities corresponding to a 3-week follow up after the toxic agent introduction the 6-week follow up after the other 6 animals revealed no simultaneous regression of the found alterations. For instance, the van Gison staining in the animals in 6 weeks after the DMNA-introduction completion showed thickened connective tissue septa with dilated venous collateral vessels filled with blood. The hematoxylin-eosin-staining revealed similar severe dystrophic liver alterations as grain and protein-hydropic dystrophy in the hepatocytes.

The 2^{nd} control group (12 rats) consisted of the animals treated with normal saline.

The daily follow-up showed that they similar to the animals of the 1^{st} control group for a long time (3-4 weeks) after the DMNA exposition completion ate badly, gained weight worse, their hair were ragged. The animals had low motional activity and looked drowsy. The morphologic and histologic examination revealed that the 3-week introduction of normal saline did not influence formation of pseudo-lobules. Despite the normal saline application there appeared hemorrhages from dilated microvessels causing alteration of lobule structure, dystrophy and death of the hepatocytes along with cell nuclei lysis. Even after the 6-week normal saline introduction the central connective tissue septa were distinct bordering the central lobules. Dilated venous collateral vessels were visible within the septa and in place of destroyed central veins. The van Gison staining revealed marked protein-hydropic dystrophy with activated fibrogenesis.

The 3^{rd} group animals were treated with GSSG·inosine that resulted in fast improvement of their state. The daily follow-up even in 7 days after onset of the treatment with the said agent exhibited enhanced physical activity, better food consumption, improved appearance and increased weight in all the rats of the said group. The histologic liver samples of the rats treated with GSSG·inosine for 3 weeks showed lowered number of the connective tissue septa, collagen fibers were distributed mainly perisinusoidally as thin threads and in lobular periportal zone. The hematoxylin-eosin staining exhibited considerable reduction in number of the cells with protein-hydropic dystrophy. Macrophagal elements were notably activated. Introduction of GSSG·inosine for 6 weeks was accompanied with significant reduction of the connective tissue amount. There were no sings of intraorganic collateral blood flow development. The given dosing at the hematoxylin-eosin stained samples was manifested with significant decrease in number of dystrophic alterations in the hepatocytes and cells with lysated nuclei. Dystrophic alterations were mainly presented as grain and hyaline-drop dystrophy in separate hepatocytes. Resorption of connective tissue septa by activated phagocytes was presented in the samples. The number of lymphocytes in the connective tissue was increased.

Observation of the animals of the 4^{th} group (12 rats) treated with Heptral indicated that for a long time (3 weeks after the treatment onset)their state did not improve: they ate badly, did not gain weight, looked badly and were sedentary. Morphologic and histologic examination revealed that after the 3-week treatment severity of acute-phase alterations was similar to the control (normal saline treatment). Pseudolobules confined by the connective tissue septa were clearly presented in the samples, they contained small collateral vessels of central veins, which anastomosed and formed links with vessels of a portal tract. The hepatocytes had severe dystrophic alterations, and protein-hydropic dystrophy was mostly present. Only 6-week dosing of Heptral resulted in slight positive effect, though less prominent than caused by the GSSG·inosine application: collagen percentage in the liver decreased, inflammatory signs alleviated and siderophages appeared in the connective tissue. Reviewing the hepatocyte status even after the 6-week dosing with Heptral there was present a large amount of cells with hydropic alteration. The aforesaid indicated insufficient efficacy of the experimental cirrhosis treatment with Heptral.

For objective estimation regarding the connective tissue content in liver of the rats of experimental and control groups fuchsinephilous (i.e. connective-tissue, collagen) fibers in the liver section were morphometrically examined. The obtained data is given in the Table 42. Treatment of toxic cirrhosis with GSSG-inosine for 3 weeks was found to be accompanied with collagen amount lowered in 1.6 times comparing to the normal saline control; at the same time, the Heptral treatment was accompanied with collagen amount decrease in the liver only in 1.1 times. After the 6-week dosing the difference was even more significant: GSSG·inosine lowered the collagen amount in 3.27 times, while Heptral·1.7 times. The aforesaid suggests that treatment with GSSG·inosine facilitates the connective tissue involution in case of liver cirrhosis. The aforesaid suggests that application of GSSG·inosine facilitates involution of the connective tissue in case of the liver cirrhosis.

Thus, high therapeutic effect of GSSG·inosine in the experimental dimethylnitrosoamine-induced liver cirrhosis model was found after daily observation after the animals confirmed with histologic examination. The liver samples of the control group (DMNA without treatment) the hepatocyte death sites, inflammation in small central vein walls and connective tissue septa formation were clearly visible. The hepatocyte exhibited dystrophic alteration, and protein dystrophy was mostly present. The normal saline administration did not have any positive outcome regarding the liver alteration: the histologic samples had mature connective tissue septa and dystrophic alterations in the hepatocytes of formed pseudolobules. The Heptral treatment brought forth slight decrease of the connective tissue amount and severity of acute-phase inflammatory processes in the portal tracts as well as dystrophic hepatocyte alterations; at that, positive effects of the Heptral administration appeared only in 6 weeks. Contrariwise, the animals treated with GSSG·inosine even in 3 weeks exhibited significant diminution of the connective tissue amount, that was more expressed after the 6-week dosing. Histologic examination of the liver samples obtained from these animals showed only solitary small connective-tissue septa containing microvessels, and any considerable protein-hydropic changes in the hepatocytes were not found indicating quite a potent hepatoprotective activity of the agent.

Conclusion. Application of GSSG·inosine dosing 10 mg/kg 3 times a week for 6 weeks for experimental dimethylnitrosamine-induced liver cirrhosis in the white rats directly correlated with the connective tissue involution in the organ and also facilitated restoration of the altered hepatocytes. The comparison agent, Heptral, exhibited considerably lower therapeutic efficacy.

TABLE 1
Effect of GSSG·inosine on outcome
of experimental Rift valley fever in white mice

| | Viral | | | | Median | |
|---|---|---|---|---|---|---|
| Drug dose | dose | | Dead/ | Survival | life-span, | Protective |
| (mg/kg) | (LD₅₀) | N | total | (%) | days | effect (%) |
| GSSG·inosine | 1-2 | 12 | 4/12 | 67 | 35.7 | 34 |
| 3 mg/kg | 10-20 | 12 | 12/12 | 0 | 7.6 | 0 |
| GSSG·inosine | 1-2 | 9 | 4/9 | 55 | 10.4 | 22 |
| 10 mg/kg | 10-20 | 9 | 7/9 | 22 | 9.0 | 22 |
| GSSG·inosine | 1-2 | 12 | 1/12 | 92 | 46.3 | 59 |
| 30 mg/kg | 10-20 | 12 | 12/12 | 0 | 7.9 | 0 |
| Ribamidil | 1-2 | 20 | 2/20 | 90 | 44.5 | 57 |
| (100 mg/kg) | 10-20 | 21 | 6/21 | 29 | 15.2 | 29 |
| Virus | 1-2 | 21 | 14/21 | 33 | 27.7 | |
| control | 10-20 | 21 | 21/21 | 0 | 8.1 | |

TABLE 2
Protective efficacy of combined administration of
GSSG·inosine and Ribamidil at experimental
Rift valley fever in white mice

| | Viral | | | | Median | |
|---|---|---|---|---|---|---|
| Drug dose | dose | | Dead/ | Survival | life-span, | Protective |
| (mg/kg) | (LD₅₀) | N | total | (%) | days | effect (%) |
| Ribamidil | 1 | 12 | 6/12 | 50 | 8.0 | 0 |
| 100 mg + | | | | | | |
| GSSG·inosine | 10 | 10 | 6/10 | 40 | 20 | 40 |
| 3 mg/kg | | | | | | |
| Ribamidil | 1 | 9 | 2/9 | 77 | 41 | 10 |
| 100 mg + | | | | | | |
| GSSG·inosine | 10 | 9 | 2/9 | 77 | 41 | 77 |
| 10 mg/kg | | | | | | |
| Ribamidil | 1 | 8 | 2/8 | 75 | 50.0 | 25 |
| 100 mg + | | | | | | |
| GSSG·inosine | 10 | 6 | 0/6 | 100 | ˜ | 100 |
| 30 mg/kg | | | | | | |
| Ribamidil | 1 | 11 | 4/11 | 54 | 16.5 | 4 |
| 100 mg/kg | 2 | 9 | 2/9 | 77 | 42.4 | 44 |
| | 10 | 12 | 2/12 | 83 | 45.2 | 83 |
| | 20 | 9 | 4/9 | 55 | 15.2 | 55 |
| GSSG·inosine, | 1 | 6 | 2/6 | 67 | 42.8 | 17 |
| 3 mg/kg | 10 | 6 | 6/6 | 0 | 8.6 | 0 |
| GSSG·inosine, | 1 | 8 | 3/8 | 62 | 40.1 | 12 |
| 10 mg/kg | 10 | 8 | 8/8 | 0 | 7.9 | 0 |
| GSSG·inosine, | 1 | 8 | 1/8 | 87 | 44.2 | 37 |
| 30 mg/kg | 10 | 8 | 7/8 | 12 | 9.7 | 12 |
| Viral | 1 | 12 | 6/12 | 50 | 27.7 | |
| Control | 2 | 9 | 6/9 | 33 | 14.8 | |
| | 10 | 12 | 12/12 | 0 | 9.0 | |
| | 20 | 9 | 9/9 | 0 | 7.8 | |

TABLE 3
Course of the Rift valley fever virus-induced infection
in white mice treated with GSSG·inosine

| | Drug | | | | | | | | | | | | | | |
|---|---|---|---|---|---|---|---|---|---|---|---|---|---|---|---|
| Gr. | dose | Cycle | | Follow-up days | | | | | | | | | | Dead/ | Survival, |
| No. | (mg/kg) | (days) | n | 1 | 2 | 3 | 4 | 5 | 6 | 7 | 8 | 9 | 10 | Total | (%) |
| 1 | 3 | 0+1+2+3+ | 12 | | | | | 1 | 1 | 1 | 1 | | | 4/12 | 66 |
| | | 4+5+6 | | | | | | | | | | | | | |
| 2 | 30 | 0+1+2+3+ | 12 | | | | 1 | 3 | 2 | 2 | 1 | | | 9/12 | 25 |
| | | 4+5+6 | | | | | | | | | | | | | |
| 3 | — | — | 16 | | | | 1 | 3 | 3 | 3 | 1 | | | 11/16 | 31 |

TABLE 4
Effect of GSSG·inosine on the white mice resistance
to the herpes simplex infecting agent

| | Immuno- | | | | |
|---|---|---|---|---|---|
| | deficiency | Virus | Animals | Dead/ | Survival |
| Group No. | type | dose, LD₅₀ | per group | Total | rate (%) |
| 1. GSSG·inosine | CPA | 30 | 7 | 7/7 | 0 |
| | | 3 | 7 | 6/7 | 14 |
| 2. GSSG·inosine | HC | 30 | 7 | 7/7 | 0 |
| | | 3 | 7 | 5/7 | 29 |
| 3. GSSG·inosine | Rad | 30 | 7 | 7/7 | 0 |
| | | 3 | 7 | 6/7 | 14 |
| 4. Cyclopherone | CPA | 30 | 7 | 7/7 | 0 |
| | | 3 | 7 | 5/7 | 29 |
| 5. Viral control | — | 30 | 7 | 7/7 | 0 |
| | | 3 | 7 | 7/7 | 0 |

TABLE 5
Effect of the GSSG·inosines on course of the
experimental horse encephalomyelitis (VHE) in white mice

| | | Agent | | Virus | | | | | |
|---|---|---|---|---|---|---|---|---|---|
| Gr. | | dose, | Regimen, | dose, | Animal | Dead/ | Mortality | Protection | T, |
| No. | Agent | mg/kg | route | LD₅₀ | number | total | rate (%) | (%) | days |
| 1 | GSSG· | 3 | −72−48−24.0 | 2 | 4* | 4/4 | 100 | 0 | 10.5 |
| | inosine | | 24.0 | | | | | | |
| | | | i/p | 1 | 5* | 2/5 | 40 | 0 | 27.7 |
| 2 | GSSG· | 3 | +2+24+48+ | 2 | 6 | 5/6 | 83 | 0 | 8.6 |
| | inosine | | 96+120 | | | | | | |
| | | | i/p | 1 | 6 | 6/6 | 100 | 0 | 7.5 |
| 3 | GSSG· | 30 | −72−48−24.0 | 2 | 6 | 5/6 | 83 | 0 | 10.2 |
| | inosine | | | | | | | | |
| | | | i/p | 1 | 6 | 0/6 | 0 | 33 | & |
| 4 | GSSG· | 30 | +2+24+48+ | 2 | 6 | 5/6 | 83 | 0 | 9.4 |
| | inosine | | 96+120 | | | | | | |
| | | | i/p | 1 | 5* | 5/5 | 100 | 0 | 7.6 |
| 5 | GSSG· | 3 | −72−48−24.0; | 2 | 5* | 1/5 | 20 | 30 | 62.5 |
| | inosine | | i/p | | | | | | |
| | +CP | 50 | −4, s/c | 1 | 6 | 1/6 | 16 | 17 | 76.9 |
| 6 | CP | 50 | −4, s/c | 2 | 6 | 3/6 | 50 | 0 | 19.6 |
| | | | | 1 | 6 | 0/6 | 0 | 33 | & |
| 7 | Control | | | 2 | 6 | 3/6 | 50 | | 14.2 |
| | | | | 1 | 6 | 2/6 | 33 | | 27.7 |

Note:
*there is marked non-specific animal death (1-2 animals per group) at first 3 days after infecting; at the infecting day there were 6 animals in all the groups.
& infinity

TABLE 6
Results on antiviral activity study of GSSG·inosine
samples on influenza virus A (H3 No. 2) in studies
on chorion-allantois membrane

| | Agent | Viral | | | | |
|---|---|---|---|---|---|---|
| | dose, | infectious titre | Neutralization | MTD¹ | MED² | |
| Agent | μg/0.5 ml | in experiment | index, Lg LD₅₀ | μg/0.5 ml | μg/0.5 ml | CTI³ |
| GSSG·inosine | 400 | 1.5 | 4.25 | 1000 | 200 | 5 |
| | 200 | 1.75 | 4.0 | | | |
| | 100 | 5.24 | 0.5 | | | |
| | 50 | 5.5 | 0.25 | | | |
| | 25 | 5.75 | 0 | | | |

¹MTD—maximal tolerant (non-toxic) dose on the CAM;
²MED—minimal effective dose;
³CTI—chemotherapeutic index (MTD to MED ratio).

TABLE 7
Increase of the Fas-receptor-enriched cell number in the liver biopsy
of the hepatitis B patients after the GSSG · inosine treatment vs.
the standard therapy

| | Stained cell distribution on 100 cells | | |
|---|---|---|---|
| Patients | 4-5 | 6-8 | 9 and more |
| n = 78 | clods per cell | clods per cell | clods per cell |
| Before treatment | 11.2 ± 0.57 | 7.1 ± 0.38 | 3.9 ± 0.21 |
| In 3 months after the | 9.7 ± 0.71 | 6.8 ± 0.36 | 4.0 ± 0.23 |
| standard treatment | | | |
| (recombinant Inter- | | | |
| feron + Lamivudine) | | | |
| n = 31 | | | |
| In 3 months after the | 10.70 ± 0.73 | 9.50 ± 0.41* | 7.3 ± 0.3* |
| GSSG · inosine | | | |
| treatment | | | |
| n = 23 | | | |

TABLE 8
Increase of the Fas-receptor-enriched cell number in the liver biopsy
of the hepatitis C patients after the GSSG · inosine treatment vs.
the standard therapy

| | Stained cell distribution on 100 cells | | |
|---|---|---|---|
| Patients | 4-5 | 6-8 | 9 and more |
| n = 54 | clods per cell | clods per cell | clods per cell |
| Before treatment | 12.4 ± 0.61 | 8.8 ± 0.47 | 5.1 ± 0.30 |
| In 6 months after the | 10.7 ± 0.60 | 9.3 ± 0.58 | 4.00 ± 0.23 |
| standard treatment | | | |
| (recombinant Inter- | | | |
| feron + Ribavirin) | | | |
| n = 21 | | | |
| In 6 months after the | 11.8 ± 0.71 | 13.9 ± 0.46* | 9.2 ± 0.43* |
| GSSG · inosine | | | |
| treatment | | | |
| n = 17 | | | |

*P < 0.05

TABLE 9
Content of Fas/APO-1/CD-95 in the cells (U/mg) of the liver biopsy
from the hepatitis B or C patients after the GSSG · inosine
treatment.

| | | In 3 (HVB) or 6 (HVC) months after |
|---|---|---|
| | | the conventional therapy/after the |
| Patients | Before treatment | GSSG · inosine treatment |
| Control (n = 6) | 1.64 ± 0.021 | |
| Hepatitis B | 1.87 ± 0.012* | 1.90 ± 0.022* |
| | (n = 16) | (n = 6)/19.6 ± 0.03* |
| | | (n = 7) |
| Hepatitis C | 1.90 ± 0.014* | 1.88 ± 0.025* |
| | (n = 24) | (n = 10)/2.28 ± 0.03* |
| | | (n = 10) |

*p < 0.05
**Control—patients where PCR did not reveal presence of the hepatitis B or C virus in blood and there is no notable fibrosis in the biopsy

TABLE 10
Effect of the oxidized glutathione composites
with nucleosides/nucleotides on the ATP-ase/helicase
activity in the lysate of the donor lymphocyte nuclei

| Concentration | | | | | |
|---|---|---|---|---|---|
| of the | | | | | |
| tested | Distribution of radioactive fractions (high-molecular/low-molecular) | | | | |
| article, | depending on amount of M13 phage DNA in the sample (pg) | | | | |
| μg/ml | 200 pg | 100 pg | 50 pg | 20 pg | 5 pg |
| — | 92 ± 7.5/ | 70 ± 4.5/ | 43 ± 2/ | 18 ± 2.2/ | 9.5 ± 0.5/91.5 ± 4.5 |
| | 8 ± 0.5 | 30 ± 2 | 57 ± 3 | 81 ± 7 | |

GSSG-I

| | | | | | |
|---|---|---|---|---|---|
| 10 | 89 ± 7/ | 72 ± 4/ | 44 ± 2/ | 19 ± 2.0/ | 10 ± 0.5/90 ± 4.5 |
| | 11 ± 2 | 28 ± 3 | 56 ± 4 | 81 ± 4 | |
| 50 | 92.5 ± 6/ | 78 ± 5/ | 51 ± 4/ | 30 ± 2.5*/ | 27 ± 1.5*/73 ± 5 |
| | 7.5 ± 1 | 22 ± 3.5 | 49 ± 5 | 70 ± 6 | |
| 100 | 92 ± 8/ | 81 ± 6/ | 53 ± 3*/ | 37 ± 3*/ | 32 ± 2*/68 ± 4 |
| | 8 ± 1 | 19 ± 1.5 | 47 ± 4 | 63 ± 6 | |

GSSG-IMP

| | | | | | |
|---|---|---|---|---|---|
| 10 | 90 ± 6/ | 74 ± 4/ | 42 ± 2.5/ | 18.5 ± 2.5/ | 10.5 ± 1/89.5 ± 6.5 |
| | 10 ± 1 | 26 ± 2 | 58 ± 4.5 | 81.5 ± 7 | |
| 50 | 91 ± 6/ | 77.5 ± 6/ | 51.5 ± 4.5/ | 31 ± 2*/ | 26 ± 2*/74 ± 5 |
| | 9 ± 1 | 22.5 ± 3 | 48.5 ± 4.5 | 69 ± 6 | |
| 100 | 92.5 ± 8/ | 81.5 ± 6/ | 54 ± 3*/ | 39.5 ± 3.5*/ | 32 ± 2*/68 ± 4 |
| | 7.5 ± 0.5 | 18.5 ± 1.5 | 46 ± 3 | 60.5 ± 6.5 | |

GSSG-UMP

| | | | | | |
|---|---|---|---|---|---|
| 10 | 92 ± 7/ | 72 ± 4.5/ | 41 ± 32/ | 20 ± 2/ | 16 ± 1/84 ± 5 |
| | 8 ± 2 | 28 ± 2.5 | 59 ± 5 | 80 ± 5 | |
| 50 | 90.5 ± 6/ | 80 ± 6/ | 59 ± 5*/ | 37.5 ± 2.5*/ | 32.5 ± 1.5*/67.5 ± 5.5 |
| | 9.5 ± 1.5 | 20 ± 3.5 | 41 ± 4 | 62.5 ± 5.5 | |
| 100 | 92.5 ± 8/ | 84 ± 6.5/ | 67 ± 5*/ | 49.5 ± 4*/ | 42 ± 3*/58 ± 4 |
| | 7.5 ± 2 | 16 ± 1.5* | 33 ± 3.5 | 50.5 ± 5 | |

*p < 0.05
GSSG-I—the oxidized glutathione compound with inosine,
GSSG-IMP—the oxidized glutathione compound with inosine-monophosphate,
GSSG-UMP—the oxidized glutathione compound with uridine-monophosphate,

GSSG-I—the oxidized glutathione compound with inosine, GSSG-IMP—the oxidized glutathione compound with inosine-monophosphate, GSSG-UMP—the oxidized glutathione compound with uridine-monophosphate

TABLE 11
Effect of the oxidized glutathione composites with
nucleosides/nucleotides on the ATP-ase/helicase activity
in the lysate of the HEP-2 cells infected with the hepatitis C virus

| | Distribution of radioactive fractions (high-molecular/low- | | | | |
|---|---|---|---|---|---|
| Concentration | molecular) depending on amount of M13 phage DNA in the sample | | | | |
| of the tested | (pg) | | | | |
| article, μg/ml | 800 | 500 | 200 | 50 | 20 |
| — | 92 ± 6/ | 75 ± 4/ | 41 ± 2.5/ | 12 ± 1/ | 10.5 ± 1/ |
| | 8 ± 0.5 | 25 ± 1.5 | 59 ± 4.5 | 88 ± 6.5 | 89.5 ± 6 |

GSSG-I

| | | | | | |
|---|---|---|---|---|---|
| 10 | 91.5 ± 5.5/ | 73.5 ± 4.5/ | 41 ± 2/ | 12.5 ± 1/ | 12 ± 1/ |
| | 8.5 ± 0.5 | 27.5 ± 1.5 | 59 ± 4 | 87.5 ± 7 | 88 ± 6 |
| 50 | 92.5 ± 6.5/ | 74 ± 5/ | 49.5 ± 3.5/ | 28 ± 2*/ | 28 ± 2.5*/ |
| | 7.5 ± 1 | 26 ± 2 | 50.5 ± 5 | 72 ± 6.5 | 72 ± 7 |
| 100 | 92.5 ± 6.5/ | 71.5 ± 5.5/ | 53.5 ± 7.5*/ | 32 ± 2.5*/ | 31.5 ± 2.5*/ |
| | 7.5 ± 1 | 28.5 ± 3 | 46.5 ± 3 | 68 ± 5.5 | 68.5 ± 6.5 |

GSSG-IMP

| | | | | | |
|---|---|---|---|---|---|
| 10 | 91 ± 6/ | 72 ± 4.5/ | 39.5 ± 3.5/ | 13.5 ± 0.5/ | 12.5 ± 1.5/ |
| | 9 ± 1 | 28 ± 1.5 | 59.5 ± 5.5 | 86.5 ± 6.5 | 87.5 ± 6.5 |
| 50 | 92 ± 7/ | 74 ± 6/ | 49.5 ± 3.5/ | 30.5 ± 2.5*/ | 29 ± 2.5*/ |
| | 8 ± 1 | 26 ± 2 | 50.5 ± 5 | 69.5 ± 6.5 | 71 ± 6.5 |
| 100 | 92.5 ± 6.5/ | 75.5 ± 7.5/ | 53.5 ± 7.5*/ | 32 ± 2*/ | 30 ± 2*/ |
| | 7.5 ± 0.5 | 25.5 ± 3.5 | 46.5 ± 3 | 68 ± 5 | 70 ± 6 |

GSSG-UMP

| | | | | | |
|---|---|---|---|---|---|
| 10 | 92.0 ± 5/ | 73.5 ± 5.5/ | 45 ± 4/ | 17.5 ± 1.5/ | 16 ± 1/ |
| | 8 ± 1 | 27.5 ± 1.5 | 55 ± 5 | 82.5 ± 8.5 | 84 ± 5 |
| 50 | 92.5 ± 6.5/ | 74 ± 5/ | 51 ± 4/ | 34 ± 3*/ | 32 ± 2.5*/ |
| | 7.5 ± 1 | 26 ± 2 | 49 ± 5 | 66 ± 7 | 78 ± 7 |
| 100 | 92.5 ± 6.5/ | 75.5 ± 6.5/ | 57.5 ± 7.5*/ | 41 ± 4*/ | 36 ± 2*/ |
| | 7.5 ± 1 | 24.5 ± 3.5 | 42.5 ± 3.5 | 59 ± 6 | 64 ± 5 |

*p < 0.05

TABLE 12
Changes of hematologic, serologic and biochemical indices before and
after the treatment with GSSG · inosine (Example 5)

| | | | | |
|---|---|---|---|---|
| | | | 1 month | 3 months |
| | Before | After | after | after |
| Indices | treatment | treatment | treatment | treatment |

Hematology

| | | | | |
|---|---|---|---|---|
| Erythrocytes (×10¹²/l) | 4.6 | 5.0 | 5.0 | 5.1 |
| Hemoglobin (g/l) | 140 | 163 | 165 | 154 |
| Leukocytes (×10⁹/l) | 6.7 | 6.5 | 5.9 | 5.2 |
| Lymphocytes, % | 28 | 32 | 24 | 24 |
| Stab neutrophils, % | 7 | 5 | 5 | 4 |
| Segmented neutrophils, | 50 | 58 | 69 | 68 |
| % | | | | |
| Monocytes, % | 10 | 5 | 3 | 4 |
| Platelets (thousand × | 160 | 252 | 197 | 206 |
| 10⁹/l) | | | | |
| Eosinophils | 5 | 0 | 1 | 0 |

Serology

| | | | | |
|---|---|---|---|---|
| Hbs Ag (ng/ml) | 180 | − | − | − |
| Anti Hbcor IgG | + | +++ | +++ | +++ |
| Anti Hbcor IgM | ++ | +++ | − | − |
| PCR HBV | + | − | − | − |
| Anti Hbs Ag | − | − | − | <10 U/ml |

Biochemistry

| | | | | |
|---|---|---|---|---|
| Bilirubin (μmole/l) | 156 | 19, 4 | 11, 3 | 12, 2 |
| ALT (U/l) | 1167 | 850 | 434 | 36 |

TABLE 13
Immune status before and after treatment with GSSG · inosine
(Example 5)

| Index | Before treatment | After treatment |
|---|---|---|
| CD4⁺ | 610 | 780 |
| CD8⁺ | 305 | 535 |
| CD4⁺/CD8⁺ | 2 | 1, 3 |
| CD16⁺ | 414 | 624 |
| CD72⁺ | 392 | 780 |
| CIC² | 168 | 119 |
| HLADR | 545 | 825 |

²circulated immune complexes

TABLE 14
Changes of the cytokine status at treatment with GSSG · inosine
(Example 5)

| Index | Before treatment (pg/ml) | After treatment (pg/ml) |
|---|---|---|
| IL-1β | 342 | 231 |
| IL-2 | 112 | 235 |
| IL-12 | 129 | 410 |
| IL-10 | 1245 | 764 |
| IFN-γ | 264 | 352 |
| IL-4 | 1751 | 531 |
| TNF-α | 761 | 431 |

TABLE 15
Changes of hematologic, serologic and biochemical indices before and
after the treatment with GSSG · inosine (Example 6)

| | | | | |
|---|---|---|---|---|
| | | | | 1 month |
| | Before | | After | after |
| Indices | treatment | In 2 weeks | treatment | treatment |

Hematology

| | | | |
|---|---|---|---|
| Erythrocytes (×10¹²/l) | 4.0 | 4.6 | 4.2 |
| Hemoglobin (g/l) | 132 | 139 | 128 |
| Leukocytes (×10⁹/l) | 5.5 | 4.8 | 4.2 |
| Lymphocytes, % | 48 | 36 | 25 |
| Stab neutrophils, % | 3 | 5 | 4 |
| Segmented neutrophils, | 42 | 69 | 68 |
| % | | | |
| Monocytes, % | 6 | 4 | 2 |
| Platelets (thousand × | 260 | 277 | 258 |
| 10⁹/l) | | | |
| Eosinophils | 1 | 1 | 2 |

Serology

| | | | | |
|---|---|---|---|---|
| Hbs Ag (ng/ml) | 180 | 168 | − | − |
| Anti Hbcor IgG | + | + | +++ | +++ |
| Anti Hbcor IgM | ++ | +++ | − | − |
| PCR HBV | + | + | − | − |
| Anti Hbs Ag (U/l) | − | − | <10 | <10 |
| PCR HBV (copies/ml) | 160000 | | <1000 | |

Biochemistry

| | | | | |
|---|---|---|---|---|
| Bilirubin (μmole/l) | 333, 0 | 52, 0 | 23, 0 | 15, 0 |
| ALT (U/l) | 2258 | 168 | 57 | 25 |
| Prothrombin index % | 60 | 80 | 86 | 84 |
| Alkaline phosphatase | 89 | | 63 | 64 |
| (U/l) | | | | |

TABLE 16
Immune status before and after treatment with GSSG · inosine
(Example 6)

| Index | Before treatment | After treatment |
|---|---|---|
| CD4⁺ | 1200 | 260 |
| CD8⁺ | 912 | 484 |
| CD4⁺/CD8⁺ | 1, 3 | 0, 53 |
| CD16⁺ | 1152 | 624 |
| CD72⁺ | 792 | 211 |
| CIC (units) | 155 | 124 |

TABLE 17
Changes of the cytokine status at treatment with GSSG · inosine
(Example 6)

| Index | Before treatment (pg/ml) | After treatment (pg/ml) |
|---|---|---|
| IL-1β | 421 | 97 |
| IL-2 | 34 | 241 |
| IL-12 | 28 | 145 |
| IL-10 | 1231 | 754 |
| IFN-γ | 213 | 561 |
| IL-4 | 1935 | 351 |
| TNF-α | 3248 | 216 |

TABLE 18
Changes of hematologic, serologic and biochemical indices before and
after the treatment with GSSG · inosine (Example 7)

| | | | | |
|---|---|---|---|---|
| | | | 1 month | 3 months |
| | Before | After | after | after |
| Indices | treatment | treatment | treatment | treatment |

Hematology

| | | | | |
|---|---|---|---|---|
| Erythrocytes (×10¹²/l) | 4.1 | 4.2 | 4.6 | 4.3 |
| Hemoglobin (g/l) | 126 | 124 | 118 | 117 |
| Leukocytes (×10⁹/l) | 4.3 | 4.6 | 4.9 | 5.1 |
| Lymphocytes, % | 25 | 29 | 31 | 28 |
| Stab neutrophils, % | 2 | 3 | 4 | 3 |
| Segmented neutrophils, | 65 | 64 | 63 | 66 |
| % | | | | |
| Monocytes, % | 3 | 4 | 2 | 1 |
| Platelets | 162 | 201 | 225 | 257 |

Serology

| | | | | |
|---|---|---|---|---|
| Hbs Ag (ng/ml) | 168 | 175 | 185 | 174 |
| Anti Hbcor IgG | +++ | +++ | +++ | +++ |
| Anti Hbcor IgM | − | − | − | − |
| PCR HBV | + | − | − | − |
| Anti Hbs Ag | <10 U/ml | <10 U/ml | <10 U/ml | <10 U/ml |

Biochemistry

| | | | | |
|---|---|---|---|---|
| Bilirubin (μmole/l) | 25.0 | 9, 0 | 13, 0 | 7, 0 |
| ALT (U/l) | 65 | 33 | 32 | 31 |

TABLE 19
Immune status before and after treatment with GSSG · inosine
(Example 7)

| Index | Before treatment | After treatment |
|---|---|---|
| CD4⁺ | 918 | 728 |
| CD8⁺ | 290 | 756 |
| CD4⁺/CD8⁺ | 3 | 1 |
| CD16⁺ | 340 | 700 |
| CD72⁺ | 476 | 344 |
| CIC³ | 180, 7 | 92 |
| HLADR | 493 | 560 |

³circulated immune complexes

TABLE 20
Changes of the cytokine status at treatment with GSSG · inosine
(Example 7)

| | | After treatment |
|---|---|---|
| Index | Before treatment (pg/ml) | (pg/ml) |
| IL-1β | 339, 5 | 108, 3 |
| IL-2 | 111 | 213 |
| IL-12 | 134 | 432 |
| IL-10 | 1250 | 577 |
| IFN-γ | 217 | 435 |
| IL-4 | 1756 | 452 |
| TNF-α | 856 | 307 |

TABLE 21
Changes of hematologic, serologic and
biochemical indices before and after the treatment with
GSSG·inosine (Example 8)

| | | | | | | | |
|---|---|---|---|---|---|---|---|
| | | 23.12. | 02.02. | 10.03. | 10.04. | 12.05. | 10.07. |
| Indices | Base-line | 97 | 98 | 98 | 98 | 98 | 98 |

Hematology

| | | | | | | | |
|---|---|---|---|---|---|---|---|
| Erythrocytes | 3.6 | 4.0 | 3.8 | 4.1 | 4.1 | 4,2 | 3,8 |
| (× 10¹²/l) | | | | | | | |
| Hemoglobin | 120 | 120 | 113 | 122 | 130 | 130 | 128 |
| (g/l) | | | | | | | |
| Leukocytes | 5.5 | 7.0 | 5.8 | 5.6 | 7.9 | 4.9 | 6.6 |
| (× 10⁹/l) | | | | | | | |
| Lymphocytes, % | 20 | 22 | 15 | 29 | 19 | 30 | 36 |
| Stab neutrophils, % | 5 | 6 | 2 | 2 | 7 | 4 | 4 |
| Segmented | 62 | 56 | 78 | 56 | 68 | 53 | 52 |
| neutrophils, % | | | | | | | |
| Monocytes, % | 8 | 10 | 4 | 10 | 4 | 8 | 3 |
| Platelets | 90 | 112 | 273 | 225 | 190 | 210 | 230 |
| (thousand × | | | | | | | |
| 10⁹/l) | | | | | | | |
| Eosinophils | 5 | 5 | — | 3 | 5 | 5 | 5 |

Serology

| | | | | | | | |
|---|---|---|---|---|---|---|---|
| Hbs Ag (ng/ml) | 163 | 180 | 148 | 115 | 120 | 169 | 140 |
| Anti Hbcor IgG | +++ | +++ | +++ | +++ | +++ | +++ | +++ |
| Anti Hbcor IgM | − | − | − | − | − | − | − |
| PCR HBV | + | + | + | + | + | − | − |
| Anti Hbs Ag | <10 | <10 | <10 | <10 | <10 | <10 | <10 |
| | U/ml | U/ml | U/ml | U/ml | U/ml | U/ml | U/ml |

Biochemistry

| | | | | | | | |
|---|---|---|---|---|---|---|---|
| Bilirubin | 46 | 19.0 | 32.0 | 36.0 | 32.0 | 27.4 | 19.7 |
| (μmole/l) | | | | | | | |
| ALT | 0.16 | 0.1 | 0.18 | 0.6 | 0.8 | 0.92 | 0.6 |
| (mmol/hr · 1) | | | | | | | |

TABLE 22
Changes of hematologic, serologic and biochemical indices before and
after the treatment with GSSG · inosine (Example 9)

| | | |
|---|---|---|
| | | Treatment |
| Indices | Baseline | completion |

Hematology

| | | |
|---|---|---|
| Erythrocytes (×10¹²/l) | 3.6 | 4.2 |
| Hemoglobin (g/l) | 120 | 126 |
| Leukocytes (×10⁹/l) | 5.5 | 4.5 |
| Lymphocytes, % | 20 | 32 |
| Stab neutrophils, % | 5 | 3 |
| Segmented neutrophils, % | 62 | 61 |
| Monocytes, % | 8 | 3 |
| Platelets (thousand × 10⁹/l) | 190 | 260 |
| Eosinophils, % | 5 | 1 |

Serology

| | | |
|---|---|---|
| Hbs Ag (ng/ml) | 163 | 161 |
| Anti Hbcor IgG | +++ | +++ |
| Anti Hbcor IgM | − | − |
| PCR HBV | − | − |
| Anti Hbs Ag | <10 U/ml | <10 U/ml |

Biochemistry

| | | |
|---|---|---|
| Bilirubin (μmol/l) | 26 | 17 |
| ALT (mmol/hr. 1) | 65 | 42 |

TABLE 23
Changes in main dopplerography parameters
before and after treatment with GSSG·inosine (Example 9)

| | Diameter (cm) | | Linear rate | | Volume rate | |
|---|---|---|---|---|---|---|
| | Before | After | Before | After | Before | After |
| | treat- | treat- | treat- | treat- | treat- | treat- |
| | ment | ment | ment | ment | tment | tment |
| Vena portae | 15 | 15 | 11.3 | 13.8 | 1137 | 1443 |
| Vena lienalis | 10 | 10 | 16.5 | 14.2 | 704 | 737 |
| Vena | 9 | 7 | 12.6 | 24.9 | 478 | 547 |
| mesenterica | | | | | | |
| superior | | | | | | |

TABLE 24
Immune status before and after treatment with GSSG · inosine
(Example 9)

| | | Before | After | |
|---|---|---|---|---|
| Index | | treatment | treatment | Norm |
| CD3+ | % | 41 | 73 | 67-76 |
| CD3+ | 10⁶/l | 610 | 1280 | 1100-1700 |
| | | | | 0.8-1.22 |
| CD4+ | % | 27 | 59 | 38-46 |
| CD4+ | 10⁶/l | 400 | 1040 | 700-1100 |
| | | | | 0.5-0.9 |
| CD8+ | % | 27 | 37 | 31-40 |
| CD8+ | 10⁶/l | 400 | 650 | 500-900 |
| | | | | 0.42-0.64 |
| CD16+/56+ | % | 9 | 14 | 10-19 |
| CD16+/56+ | 10⁶/l | 130 | 321 | 200-400 |
| CD25+ | % | 11 | 29 | 13-24 |
| CD25+ | 10⁶/l | 160 | 650 | 208-576 |
| | | | | 0.21-0.58 |
| CD20+ | % | 12 | 17 | 11-16 |
| CD20+ | 10⁶/l | 180 | 300 | 200-400 |
| | | | | 0.20-0.40 |
| HLA-DR | % | 26 | 45 | 19-30 |
| HLA-DR | 10⁶/l | 380 | 790 | 304-720 |
| | | | | 0.34-0.72 |

TABLE 25
Changes of hematologic, serologic and biochemical indices before and
after the treatment with GSSG · inosine (Example 10)

| | | | | |
|---|---|---|---|---|
| | | | 1 month | 3 months |
| | Before | After | after | after |
| Indices | treatment | treatment | treatment | treatment |

Hematology

| | | | | |
|---|---|---|---|---|
| Erythrocytes (×10¹²/l) | 4.1 | 4.0 | 4.4 | 4.6 |
| Hemoglobin (g/l) | 120 | 140 | 136 | 148 |
| Leukocytes (×10⁹/l) | 8.9 | 6.7 | 5.7 | 5.8 |
| Lymphocytes, % | 30 | 47 | 46 | 17 |
| Stab neutrophils, % | 5 | 2 | 1 | 2 |
| Segmented | 52 | 41 | 38 | 68 |
| neutrophils, % | | | | |
| Monocytes, % | 10 | 8 | 11 | 12 |
| Platelets (thousand × | 240 | 280 | 215 | 265 |
| 10⁹/l) | | | | |
| Eosinophils | 3 | 2 | 4 | 1 |

Serology

| | | | | |
|---|---|---|---|---|
| Hbs Ag (ng/ml) | − | − | − | − |
| Anti Hbcor IgG | +++ | +++ | +++ | +++ |
| Anti Hbcor IgM | − | − | − | − |
| PCR HCV | + | − | − | − |
| Anti Hbs Ag | 10 U/ml | 10 U/ml | 75 U/ml | 75 U/ml |
| Anti HCV IgG | +++cor | +++cor | ++cor++ns | ++cor++ns |
| Bilirubin (μmole/l) | 34.0 | 22.0 | 24.0 | 18.0 |
| ALT (U/l) | 86 | 41 | 39 | 40 |

TABLE 26
Immune status before and after treatment with GSSG · inosine
(Example 10)

| Index | Before treatment | After treatment |
|---|---|---|
| CD4⁺ | 519 | 679 |
| CD8⁺ | 541 | 450 |
| CD4⁺/CD8⁺ | 1 | 1.23 |
| CD4⁺ CD8⁺ | 363 | 256 |
| CD16⁺ | 573.7 | 358 |
| CD72⁺ | 676 | 459 |
| CIC⁴ | 180.7 | 92 |
| HLADR | 715 | 424 |
| CD95 + (FasAg) | 5 | 45 |

⁴circulated immune complexes

TABLE 27
Changes of the cytokine status at treatment with GSSG · inosine
(Example 10)

| | Before treatment | After treatment |
|---|---|---|
| Index | (pg/ml) | (pg/ml) |
| IL-1β | 239.5 | 108.3 |
| IL-2 | 128 | 156 |
| IL-12 | 156 | 250 |
| IL-10 | 1133 | 887 |
| IFN-γ | 307.9 | 389 |
| IL-4 | 1800 | 600 |
| TNF-α | 976 | 358 |

TABLE 28
Changes of hematological, serologic and biochemical indices prior
to and after treatment with GSSG · inosine (Example 11)

| | | |
|---|---|---|
| Indices | Baseline | Treatment completion |

Hematology

| | | |
|---|---|---|
| Erythrocytes (10⁶/mm³) | 4, 6 | 3, 95 |
| Hemoglobin (G/l) | 125 | 13, 3 |
| Leukocytes (10³/mm³) | 9, 1 | 4, 8 |
| Lymphocytes - % | 6 | 29 |
| Stab neutrophils - % | 6 | 3 |
| Segmented neutrophils - % | 78 | 59 |
| Monocytes - % | 3 | 7 |
| Platelets (10³/mm³) | 141 | 211 |
| Eosinophils - % | 0 | 2 |

Serology

| | | |
|---|---|---|
| HBsAg (ng/ml) | − | − |
| anti-HCV-IgG | + | + |
| HCV-RNA | − | − |

Biochemistry

| | | |
|---|---|---|
| Bilirubin (μm/l) | 31 | 17 |
| ALT (mmol/hr. 1.) | 0, 44 | 0, 34 |
| Alkaline phosphatase (U/l) | 380 | 261 |
| Creatinine (μm/l) | 260 | 154 |
| Lactate dehydrogenase (U/l) | 420 | 210 |
| Prothrombin - % | 72 | 86 |

TABLE 29
Development of major dopplerographic indices
prior to and after the treatment with
GSSG·inosine (Example 11)

| | | | | | Volume rate | |
|---|---|---|---|---|---|---|
| | | | Linear rate (cm/s) | | (ml/min) | |
| | Diameter(cm) | | Before | After | Before | After |
| | Before | After | treat- | treat- | treat- | treat- |
| | treatment | treatment | ment | ment | ment | ment |
| Vena portae | 12 | 12 | 9.7 | 14.8 | 633 | 1029 |
| Vena lienalis | 9 | 9 | 6.6 | 13.8 | 212 | 500 |
| Vena | 11 | 11 | 5.4 | 8.0 | 297 | 450 |
| mesenterica | | | | | | |
| superior | | | | | | |
| Splenic-renal | Grade 1 | Not | | | | |
| anastomoses | | found | | | | |
| Spleen (cm) | 12.6 × 7.0 | 12.5 × 3.0 | | | | |

TABLE 30
General status valuation - Karnovsky scale (Example 11)

| Before the | | | | |
|---|---|---|---|---|
| treatment | 1 weeks | 2 weeks | 3 weeks | 4 weeks |
| onset | later | later | later | later |
| 30% | 35% | 35% | 40% | 60% |
| 2 months | 3 months | 6 months | 9 months | 12 months |
| later | later | later | later | later |
| 55% | 60% | | | |

TABLE 31
Blood and immune indices (Example 12)

| IMMUNE | | | 14.04. | 15.05. | |
|---|---|---|---|---|---|
| BLOOD TEST | 28.02.96 | 20.03.96 | 96 | 96 | Norm |
| Lymphocytes | 570 | 832 | 736 | 1350 | 1200-3000 |
| CD20+ | 0 | 150 | 125 | 419 | 200-400 |
| CD4+ | 182 | 175 | 199 | 527 | 700-1100 |
| CD8+ | 257 | 208 | 228 | 608 | 500-900 |
| CD4/CD8 | 0.71 | 0.84 | 0.87 | 0.87 | 1.0-1.5 |
| CD25+ | 0 | 191 | 109 | 513 | 208-576 |
| HLA-DR | 0 | 241 | 346 | 756 | 304-720 |
| CD16 | 0 | 175 | 309 | 540 | 200-400 |
| IgA | 5.4 | 3.4 | 3.7 | 4.2 | 0.8-5.2 |
| IgM | 3.2 | 2 | 3.4 | 2.6 | 0.6-3.8 |
| IgG | 23 | 18 | 22 | 26 | 6.0-18.0 |
| CIC⁵ | 0.28 | 0.27 | 0.16 | 0.17 | 0.03-0.08 |
| GENERAL | | | 03.04. | 12.04. | |
| BLOOD TEST | 28.02.96 | 20.03.96 | 96 | 96 | Norm |
| Erythrocytes | 3.7 | 3.4 | 3.2 | 3.8 | 4.0-5.0 |
| Hemoglobin | 117 | 108 | 100 | 128 | 130-160 |
| Platelets | 170 | 170 | 150 | 120 | 180-320 |
| Leukocytes | 1.9 | 3.2 | 2.2 | 1.8 | 4.0-9.0 |
| Stab | 3 | 12 | 3 | 5 | 1-6 |
| neutrophils | | | | | |
| Segmented | 57 | 53 | 51 | 50 | 47-72 |
| neutrophils | | | | | |
| Lymphocytes | 30 | 26 | 36 | 39 | 19-37 |
| Monocytes | 2 | 4 | 2 | 5 | 3-11 |
| ESR⁶ | 50 | 17 | 63 | 31 | 2-10 |

TABLE 32
Viral load development at treatment with GSSG · inosine
(D-form) (Example 12)

| | | 1-month | 2-month | Treatment |
|---|---|---|---|---|
| Index | Initial value | treatment | treatment | completion |
| Viral load | 110000 | 89000 | 56000 | 10000 |
| (copies of HIV | | | | |
| RNA/ml) | | | | |

⁵circulated immune complexes
⁶erythrocyte sedimentation rate

TABLE 33
Blood and immune indices (Example 13)

| IMMUNE | | | | | |
|---|---|---|---|---|---|
| BLOOD TEST | 13.03.96 | 03.04.96 | 17.04.96 | 15.05.96 | Norm |
| Lymphocytes | 984 | 988 | 1276 | 2295 | 1200-3000 |
| (CD20+) | 256 | 217 | 306 | 390 | 200-400 |
| (CD4+) | 276 | 247 | 319 | 574 | 700-1100 |
| (CD8+) | 354 | 277 | 370 | 597 | 500-900 |
| (CD4/CD8) | 0.78 | 0.89 | 0.86 | 0.96 | 1.0-1.5 |
| (CD25+) | 226 | 207 | 332 | 482 | 208-576 |
| HLA-DR | 354 | 237 | 434 | 665 | 304-720 |
| (CD16) | 216 | 198 | 332 | 505 | 200-400 |
| IgA | 1.8 | 2.1 | 1.6 | 1.5 | 0.8-5.2 |
| IgM | 2.6 | 4.6 | 5.2 | 3.1 | 0.6-3.8 |
| IgG | 18.8 | 20 | 24 | 28 | 6.0-18.0 |
| CIC⁷ | 0.09 | 0.175 | 0.11 | 0.075 | 0.03-0.08 |

| GENERAL | 13.03. | 28.03. | 01.04. | 03.04. | 16.04. | 15.05. | |
|---|---|---|---|---|---|---|---|
| BLOOD TEST | 96 | 96 | 96 | 96 | 96 | 96 | Norm |
| Erythrocytes | 5 | 4.3 | 3.9 | 3.9 | 4 | 4.2 | 3.9-4.7 |
| Hemoglobin | 160 | 136 | 126 | 125 | 129 | 136 | 120.0-140.0 |
| Platelets | 200 | | 180 | 170 | 180 | 190 | 180.0-320.0 |
| Leukocytes | 2.4 | 2.9 | 3.5 | 2.6 | 2.9 | 4.5 | 4.0-9.0 |
| Stab | 5 | 4 | 3 | 8 | 8 | 7 | 1-6 |
| neutrophils | | | | | | | |
| Segmented | 48 | 35 | 41 | 47 | 45 | 49 | 47-72 |
| neutrophils | | | | | | | |
| Lymphocytes | 41 | 51 | 49 | 38 | 44 | 41 | 19-37 |
| Monocytes | 2 | 2 | 2 | 7 | 2 | 3 | 3-11 |
| ESR | 18 | 16 | | 21 | 20 | 12 | 2-15 |

TABLE 34
Viral load development at treatment with Zn₂-GSSG-TMP
(Example 13)

| | | | Treatment |
|---|---|---|---|
| Index | Initial value | 1-month treatment | completion |
| Viral load | 80000 | 66000 | 15000 |
| (copies of HIV | | | |
| RNA/ml) | | | |

TABLE 35
Major laboratory indices development (Example 14)

| | | | 1 | 3 | |
|---|---|---|---|---|---|
| | | | month | months | |
| | Before | After | after | after | |
| | treat- | treat- | treat- | treat- | |
| Indices | ment | ment | ment | ment | Norm |
| Chlamydia in ejaculate | +++ | − | − | − | − |
| (DIFA, cell culture) | | | | | |
| Chlamydia in prostate | +++ | − | − | − | − |
| secretion (DIFA, cell | | | | | |
| culture) | | | | | |
| Chlamydia in synovial | ++ | − | − | − | − |
| fluid (DIFA, cell culture) | | | | | |
| Chlamydia in blood | ++ | − | − | − | − |
| leukocytes (DIFA, cell | | | | | |
| culture) | | | | | |
| Infectious agent in | ++ | + | ± | − | − |
| prostate secretion (PCR) | | | | | |
| Infectious agent in | ++ | ± | − | − | − |
| synovial fluid (PCR) | | | | | |
| CD3 (T-lymphocytes), % | 52 | 59 | 68 | 72 | 60-85 |
| CD4 (T-lymphocytes), % | 26 | 29 | 32 | 40 | 31-46 |
| CD8 (T-lymphocytes), % | 22 | 24 | 30 | 32 | 26-40 |
| CD20 (B-lymphocytes), % | 18 | 16 | 15 | 16 | 11-16 |
| IL-1 (1β), pg/ml | 100 | 250 | 60 | 0 | 0 |
| IL-2, un/pg | 20 | 44 | 62 | 80 | 0-40 |
| IL-8, pg/ml | 340 | 120 | 64 | 0 | 0 |
| IFN-α, IU/ml | 52 | 102 | 94 | 108 | 0-8 |
| IFN-γ, IU/ml | 37 | 97 | 93 | 314 | 10-60 |

TABLE 36
Development of major laboratory indices (Example 15)

| | | After | After | |
|---|---|---|---|---|
| | | 1- | 3- | |
| | Value | month | month | |
| | before | treat- | treat- | |
| Indices | treatment | ment | ment | Norm |
| Chlamydia by smear (IFA) | + | − | − | − |
| Serum antibody titer - | 1:232 | − | − | Io 1:32 |
| IgG | | | | |
| PCR for Chlamydia | + | − | − | − |
| trachomatis | | | | |
| PCR for Herpes simplex | + | − | − | − |
| I + II | | | | |
| T-lymphocytes (CD3+), % | 50 | 65 | 73 | 60-85 |
| T-lymphocytes (CD3+), | 675 | 1623 | 1601 | 590-2200 |
| 10⁶/ml | | | | |
| B-lymphocytes (CD19+), % | 13 | 12 | 9 | 5-20 |
| B-lymphocytes (CD19+), | 176 | 300 | 212 | 95-532 |
| 10⁶/ml | | | | |
| T-helpers (CD4+), % | 29 | 62 | 61 | 30-60 |
| T-helpers (CD4+), 10⁶/ml | 392 | 1005 | 979 | 309-1571 |
| T-cytotoxic lymphocytes | 22 | 35 | 33 | 19-48 |
| (CD8+), % | | | | |
| T-cytotoxic lymphocytes | 297 | 562 | 532 | 282-999 |
| (CD8+), 10⁶/ml | | | | |
| IL-2 receptor carrying cells | 149 | 429 | 433 | 208-576 |
| (CD25+), 10⁶/ml | | | | |
| HLA (II) DR receptor, | 243 | 374 | 383 | 304-720 |
| 10⁶/ml | | | | |
| NK-K$$eTKH (CD16+), | 270 | 407 | 386 | 78-605 |
| 10⁶/ml | | | | |
| LMIT* - Spontaneous | 1.2 | 4.4 | 4.0 | 2.5-5.0 |
| leukocyte migration, un. | | | | |
| LMIT - | 33 | 52 | 48 | 29-56 |
| phytohemagglutinin, % | | | | |
| LMIT - concavalin A, % | −116 | 42 | 65 | 40-75 |
| Phagocyte number, % | 46 | 88 | 72 | 65-95 |
| Phagocyte index | 4 | 6 | 7 | 4-10 |
| Phagocytosis completion | 0.7 | 1.6 | 1.6 | >1 |
| index | | | | |
| NBT** - basal test, % | 1 | 8 | 8 | 6-10 |
| NBT - stimulated test, % | 37 | 76 | 74 | 40-80 |
| Total serum IFN, IU/ml | 26 | 42 | 8 | 0-8 |
| IFN - α/β | 66 | 564 | 432 | 250-520 |
| IFN - γ | 32 | 265 | 186 | 110-250 |

*LMIT - Leukocyte migration-inhibition test
**NBT - nitroblue tetrazolium

TABLE 37
Development of major laboratory indices (Example 16)

| | | | | |
|---|---|---|---|---|
| | | 19.12. | 21.01. | |
| 20.10.98 | 21.11.98 | 98 | 99 | Norm |

BLOOD IMMUNE TEST

| | | | | | |
|---|---|---|---|---|---|
| Lymphocytes | 832 | 1350 | 1400 | 1680 | 1200-3000 |
| CD4+ | 199 | 527 | 860 | 892 | 700-1100 |
| CD8+ | 357 | 422 | 563 | 594 | 500-900 |
| CD4/CD8 | 0.56 | 1.25 | 1.53 | 1.50 | 1.0-1.5 |
| IL-1 | 22 | 29 | 46 | 39 | 30-50 |
| IL-4 | 32 | 30 | 37 | 34 | 30-50 |
| IL-6 | 16 | 29 | 44 | 39 | 30-50 |
| IL-8 | 23 | 45 | 50 | 48 | 30-100 |
| IL-12 | 14 | 28 | 39 | 47 | 30-50 |

GENERAL BLOOD TEST

| | | | | | |
|---|---|---|---|---|---|
| Erythrocytes | 3.7 | 4.2 | 4.3 | 4.2 | 4.0-5.0 |
| Hemoglobin | 108 | 128 | 132 | 128 | 130-160 |
| Platelets | 260 | 210 | 230 | 210 | 180-320 |
| Leukocytes | 9.9 | 5.2 | 5.2 | 6.8 | 4.0-9.0 |
| Stab | 12 | 6 | 1 | 3 | 1-6 |
| neutrophils | | | | | |
| Segmented | 57 | 53 | 51 | 50 | 47-72 |
| neutrophils | | | | | |
| Monocytes | 12 | 4 | 2 | 5 | 3-11 |
| ESR | 50 | 27 | 13 | 11 | 2-10 |

TABLE 38
Morphometric indices characterizing liver state of the animals
introduced with Dichlorethane and Acetaminophen
(20^{th} Study Day), M ± m (Example 17)

| | Control | | Experiment |
|---|---|---|---|
| Indices and | (intact | Experiment (no | (treatment with |
| measurement units | animals) | treatment) | GSSG · inosine) |
| Weight, g | 190 ± 5 | 130 ± 10* | 180 ± 15** |
| Relative liver weight, | 27.7 ± 1.1 | 56.2 ± 1.2* | 31.3 ± 1.3** |
| mg/100 g of weight | | | |

*significant differences from the control (p < 0.05)
**significant differences from the untreated experimental group

TABLE 39
Biochemical indices characterizing liver state of the animals
introduced with Dichlorethane and Acetaminophen
(20^{th} Study Day), M ± m (Example 17)

| | Control | | Experiment |
|---|---|---|---|
| Indices and | (intact | Experiment (no | (treatment with |
| measurement units | animals) | treatment) | GSSG ∞ inosine) |
| 1 | 2 | 3 | 4 |

Protein-synthesizing function

| | | | |
|---|---|---|---|
| Total serum protein, | 64 ± 2 | 28 ± 4* | 52 ± 5** |
| g/l | | | |
| y-GTP, μcat/l | 0.86 ± 0.10 | 0.21 ± 0.07* | 0.72 ± 0.09** |
| Prothrombin time, | 33 ± 5 | 92 ± 17* | 43 ± 5** |
| serum, s | | | |

Lipid metabolism

| | | | |
|---|---|---|---|
| Total lipids, g/l | 3.7 ± 0.3 | 2.1 ± 0.1* | 3.2 ± 0.1** |
| Cholesterol, mmole/l | 1.72 ± 0.44 | 0.82 ± 0.24* | 1.58 ± 0.21** |

Cytolytic syndrome

| | | | |
|---|---|---|---|
| ALT, μcat/l | 0.20 ± 0.03 | 2.55 ± 0.25* | 0.54 ± 0.08** |
| AST, μcat/l | 0.60 ± 0.05 | 1.54 ± 0.17* | 0.82 ± 0.06** |
| Acid phosphatase, | 0.74 ± 0.12 | 3.07 ± 0.17* | 0.82 ± 0.03** |
| μcat/l | | | |

Carbohydrate metabolism

| | | | |
|---|---|---|---|
| Glycogen, liver, mg % | 2500 ± 100 | 400 ± 60* | 1600 ± 100** |
| Glucose, serum, | 5.0 ± 0.3 | 3.2 ± 0.4* | 4.2 ± 0.4 |
| mmole/l | | | |

Cholestasis

| | | | |
|---|---|---|---|
| Total bilirubin, serum, | 3.0 ± 0.3 | 8.2 ± 0.2* | 3.2 ± 0.1** |
| mmole/l | | | |
| Alkaline phosphatase, | 0.69 ± 0.10 | 3.15 ± 0.35* | 0.72 ± 0.09** |
| μcat/l | | | |

*significant differences from the control (p < 0.05)
**significant differences from the untreated experimental group

TABLE 40
Effect of GSSG · inosine on liver detoxicating function in the animals
exposed to Dichlorethane and Acetaminophen
(20^{th} Study Day), M ± m (Example 17)

| | | | Experiment |
|---|---|---|---|
| | Control | Experiment | (treatment with |
| Indices and | (intact | (no | GSSG · |
| measurement units | animals) | treatment) | inosine) |
| Reduced glutathione, | 160 ± 5 | 50 ± 10* | 165 ± 10** |
| liver, mg % | | | |
| —SH_{gr}, blood, μmole/ | 1650 ± 90 | 330 ± 30* | 1100 ± 50** |
| 100 ml | | | |
| Cytochrome P₄₅₀, | 1.24 ± 0.03 | 0.62 ± 0.06* | 1.08 ± 0.06** |
| liver, mmole/mg of | | | |
| protein × 10⁻⁴ | | | |
| Cytochrome B₅, liver, | 0.85 ± 0.04 | 0.34 ± 0.06* | 0.64 ± 0.07** |
| mmole/mg of | | | |
| protein × 10⁻⁴ | | | |
| Bromine—sulfaleine | 13.86 ± 1.2 | 54.2 ± 2.6* | 19.3 ± 1.1** |
| content, serum, in | | | |
| 10 minutes after | | | |
| introduction, mg % | | | |
| Hexenal sleep, min | 25.0 ± 1.5 | 52.5 ± 2.5* | 27.0 ± 1.5** |

*significant differences from the control (p < 0.05)
**significant differences from the untreated experimental group

TABLE 41
Rat weight development during the
Study (g) (Example 18)

| | | | 1 | 3 | 6 |
|---|---|---|---|---|---|
| | | After | treatment | treatment | treatment |
| Group | Baseline | poisoning | week | weeks | weeks |
| Intact | 150.5 | 182.0 | 237.5 | 251.25 | 262 |
| Group No. 1, | | 156.25 | 178.75 | 195 | 211 |
| DMNA | | | | | |
| Group No. 2, | | | 184 | 200 | 223 |
| normal | | | | | |
| saline | | | | | |
| Group No. 3, | | | 200.6 | 238 | 259 |
| GSSG• | | | | | |
| inosine | | | | | |
| Group No. 4, | | | 191.0 | 212 | 232 |
| Heptral | | | | | |

TABLE 42
Connective tissue amount in liver parenchyma of the white rats being
treated for experimental dimethylnitrosamine(DMNA)-induced
cirrhosis (Example 18)

| | | Relative square of |
|---|---|---|
| | | the connective tissue |
| Treatment duration | Treatment variant | in a liver section, % |
| Intact animals | | 1.744 ± 0.101 |
| 3 weeks after cirrhosis | No treatment (only DMNA) | 8.654 ± 0.702 |
| modeling completion | Normal saline | 9.292 ± 0.886 |
| | GSSG · inosine | 5.74 ± 0.421* |
| | Heptral | 8.215 ± 0.751 |
| 6 weeks after cirrhosis | No treatment (only DMNA) | 15.840 ± 1.476 |
| modeling completion | Normal saline | 13.288 ± 1.221 |
| | GSSG · inosine | 4.062 ± 0.320* |
| | Heptral | 7.781 ± 0.943* |

Note:
*significant difference with normal saline-treated animals, p < 0.05.

TABLE 43
ALT in Acute HBV Infection (normal range: 0-41 U/L),
treatment with the Tested Article

| | | Symptomatic Therapy |
|---|---|---|
| Months | GSSG · inosine n = 20 | n = 19 |
| Baseline (before | 651 ± 18.5 | 604 ± 20.0 |
| treatment) | | |
| 1 (after treatment) | 153.5 ± 9.5* | **350.8 ± 3.5* |
| 2 (1 month after | 56.4 ± 5.0* | **115.3 ± 2.2* |
| treatment)*** | | |
| 4 (3 months after | 31.0 ± 3.7* | **75.5 ± 6.3* |
| treatment)*** | | |

*statistically significant as per examination terms;
**statistically significant comparing to the experimental group.
***no additional treatment was applied.

TABLE 44
PCR (%) in Acute HBV Infection, treatment with the Tested Article

| | GSSG · inosine | Symptomatic |
|---|---|---|
| Months | n = 20 | Therapy n = 19 |
| Baseline (before treatment) | 100 | 100 |
| 1 (after treatment) | 20* | **57.9* |
| 2 (1 month after treatment)*** | 0* | **42.0* |
| 4 (3 months after treatment)*** | 0 | **57.9 |

*statistically significant as per examination terms;
**statistically significant comparing to the experimental group.
***no additional treatment was applied.

TABLE 45
HbsAg (ng/mL) in Acute HBV Infection, treatment with the
Tested Article

| | GSSG · inosine | Symptomatic Therapy |
|---|---|---|
| Months | n = 20 | n = 19 |
| Baseline (before treatment) | 159.8 ± 12.9 | 172.0 ± 4.6 |
| 1 (after treatment) | 49.5 ± 24.8* | **136.7 ± 2.3* |
| 2 (1 month after treatment)*** | 11.2 ± 3.5* | **87.3 ± 2.7* |
| 4 (3 months after treatment)*** | 0* | 68.8 ± 3.8 |

*statistically significant as per examination terms;
**statistically significant comparing to the experimental group.
***no additional treatment was applied.

TABLE 46
Bilirubin in Acute HBV Infection (normal range: 8-20.5 μmole/L),
treatment with the Tested Article

| | GSSG · inosine | Symptomatic Therapy |
|---|---|---|
| Months | n = 20 | n = 19 |
| Baseline (before treatment) | 79.21 ± 28.8 | 82.7 ± 1.5 |
| 1 (after treatment) | 26.2 ± 8.0* | **46.7 ± 2.7* |
| 2 (1 month after treatment)*** | 21.8 ± 7.6 | 27.6 ± 3.0* |
| 4 (3 months after treatment)*** | 16.5 ± 6.6* | **22.0 ± 3.2* |

*statistically significant as per examination terms;
**statistically significant comparing to the experimental group.
***no additional treatment was applied

TABLE 47
ALT in Chronic HBV Infection (normal range: 0-41 U/L),
treatment with the Tested Article

| | | Famvir | Invirasa |
|---|---|---|---|
| | | (Famciclovir) | (Sacvinavir) |
| | GSSG · inosine* | Glaxo | Hoffman La |
| Months | n = 22 | Wellcome | Roche |
| Baseline (before | 196.9 ± 9.8 | 165.7 ± 13.8 | 165.7 ± 17.0 |
| treatment) | | | |
| 1 (after treatment) | 55.1 ± 6.7* | **70.2 ± 9.1* | **90.8 ± 5.5 |
| 2 (1 month after | 31.6 ± 1.9* | **75.9 ± 9.6 | **71.4 ± 6.5 |
| treatment)*** | | | |
| 4 (3 months after | 37.5 ± 3.5 | **77.9 ± 10.2 | **71.8 ± 7.4 |
| treatment)*** | | | |

*statistically significant as per examination terms;
**statistically significant comparing to the experimental group.
***no additional treatment was applied.

TABLE 48
HbsAg (ng/mL) in Chronic HBV Infection,
treatment with the Tested Article

| | | | Invirasa |
|---|---|---|---|
| | | Famvir | (Sacvinavir) |
| | | (Famciclovir) | Hoffman La |
| | GSSG · inosine* | Glaxo Wellcome | Roche |
| Months | n = 22 | n = 20 | n = 20 |
| Baseline | 123.21 ± 32.55 | 157.8 ± 8.84 | 137.0 ± 20.6 |
| (before | | | |
| treatment) | | | |
| 1 (after | 90.26 ± 33.65* | **150.5 ± 10.43 | **109.0 ± 54.5* |
| treatment) | | | |
| 2 (1 month | 73.56 ± 35.39* | **153.5 ± 10.14 | **112.2 ± 22.2 |
| after | | | |
| treatment)*** | | | |
| 4 (3 months | 67.87 ± 35.41 | **132.5 ± 43.76 | **172.0 ± 14.5* |
| after | | | |
| treatment)*** | | | |

*statistically significant as per examination terms;
**statistically significant comparing to the experimental group.
***no additional treatment was applied.

TABLE 49
PCR (%) in Chronic HBV Infection

| | | | Invirasa |
|---|---|---|---|
| | | Famvir | (Sacvinavir) |
| | | (Famciclovir) | Hoffman La |
| | GSSG · inosine* | Glaxo Wellcome | Roche |
| Months | n = 22 | n = 20 | n = 20 |
| Baseline (before | 100 | 100 | 100 |
| treatment) | | | |
| 1 (after treatment) | 13.64 | **50.0* | **70.0* |
| 2 (1 month after | 13.64 | **60.0 | **60.0* |
| treatment)*** | | | |
| 4 (3 months after | 9.09 | **20.0 | **60.0 |
| treatment)*** | | | |

*statistically significant as per examination terms;
**statistically significant comparing to the experimental group.
***no additional treatment was applied.

TABLE 50
ALT in Severe HBV Infection (normal range: 0-41 U/L),
treatment with the Tested Article

| | | Symptomatic Therapy and |
|---|---|---|
| | | Prednisolone (500- |
| | GSSG · inosine | 1000 mg per treatment |
| Months | n = 22 | course) n = 15 |
| Baseline (before | 1593.71 ± 217.8 | 1657.9 ± 96.5 |
| treatment) | | |
| 1 (after treatment) | 113.6 ± 26.3* | **293.8 ± 18.8* |
| 2 (1 month after | 40.5 ± 5.86* | **145.4 ± 15.5* |
| treatment)*** | | |
| 4 (3 months after | 29.6 ± 4.7* | **65.3 ± 15.7* |
| treatment)*** | | |

*statistically significant as per examination terms;
**statistically significant comparing to the experimental group.
***no additional treatment was applied.

TABLE 51
HbsAg (ng/mL) in Severe HBV Infection,
treatment with the Tested Article

| | | Symptomatic Therapy and |
|---|---|---|
| | GSSG · inosine* | Prednisolone (500-1000 mg per |
| Months | n = 20 | treatment course) n = 15 |
| Baseline (before | 184.57 ± 18.90 | 189.7 ± 20.4 |
| treatment) | | |
| 1 (after treatment) | 21.28 ± 3.7* | **97.8 ± 18.6* |
| 2 (1 month after | 7.54 ± 1.24* | **87.7 ± 12.7 |
| treatment)*** | | |
| 4 (3 months after | 0 | **76.6 ± 15.4 |
| treatment) | | |

*statistically significant as per examination terms;
**statistically significant comparing to the experimental group.
***no additional treatment was applied.

TABLE 52
PCR (%) in Severe HBV Infection, treatment with the Tested Article

| | | Symptomatic Therapy and |
|---|---|---|
| | | Prednisolone (500- |
| | GSSG · inosine* | 1000 mg per treatment |
| Months | n = 20 | course) n = 15 |
| Baseline (before | 100 | 100 |
| treatment) | | |
| 1 (after treatment) | 10* | **66.7 |
| 2 (1 month after | 5 | **66.7 |
| treatment)*** | | |
| 4 (3 months after | 0 | **53.3 |
| treatment)*** | | |

*statistically significant as per examination terms;
**statistically significant comparing to the experimental group.
***no additional treatment was applied.

TABLE 53
Bilirubin in Severe HBV Infection (normal range: 8-20.5 μmole/L),
treatment with the Tested Article

| | | |
|---|---|---|
| | | Symptomatic Therapy and |
| | GSSG · inosine* | Prednisolone (500-1000 mg |
| Months | n = 20 | per treatment course) n = 15 |

| | | |
|---|---|---|
| Baseline (before | 306.5 ± 82.4 | 313.9 ± 62.6 |
| treatment) | | |
| 1 (after treatment) | 43.8 ± 10.7* | **86.9 ± 21.9* |
| 2 (1 month after | 18.95 ± 2.4* | **31.4 ± 14.6* |
| treatment)*** | | |
| 4 (3 months after | 14.92 ± 2.73 | **26.84 ± 13.99 |
| treatment)*** | | |

*statistically significant as per examination terms.
**statistically significant comparing to the experimental group.
***no additional treatment was applied.

TABLE 54
ALT in Chronic HCV Infection (normal range: 0-41 U/L),
treatment with the Tested Article

| | | | Hivid |
|---|---|---|---|
| | | Retrovir | (Zalzitabin) |
| | | (Zidovudine) | Hoffman La |
| | GSSG · inosine | Glaxo Wellcome | Roche |
| Months | n = 29 | n = 22 | n = 23 |
| Baseline (before | 239.5 ± 42.5 | 180 ± 20.0 | 187.5 ± 48.4 |
| treatment) | | | |
| 1 (after treatment) | 60 ± 11.8 | **132.0 ± 51.5* | **190 ± 71.1* |
| 2 (1 month after | 54.0 ± 9.6 | **108 ± 40.3* | **153 ± 25.3* |
| treatment)*** | | | |
| 4 (3 months after | 59.5 ± 10.3 | **90.0 ± 25.2* | **151 ± 25.2* |
| treatment)*** | | | |

*statistically significant as per examination terms.
**statistically significant comparing to the experimental group.
***no additional treatment was applied.

TABLE 55
PCR (%) in Chronic HCV Infection, treatment with the Tested Article

| | GSSG · inosine | Zidovudine | Zalzitabin |
|---|---|---|---|
| Months | n = 29 | N = 22 | N = 23 |
| Baseline (before | 100 | 100 | 100 |
| treatment) | | | |
| 1 (after treatment) | 37.93 | **54.5* | **73.9* |
| 2 (1 month after | 41.4 | **63.6* | **56.5* |
| treatment)*** | | | |
| 4 (3 months after | 41.4 | **59.1* | **60.9* |
| treatment)*** | | | |

*statistically significant as per examination terms;
**statistically significant comparing to the experimental group.
***no additional treatment was applied.

What is claimed is:

1. A composition comprising an oxidized glutathione salt comprising oxidized glutathione and at least one counterion of the oxidized glutathione comprising a nitrogenous base selected from DNA bases, nucleosides of DNA bases, nucleotides of DNA bases, RNA bases, nucleosides of RNA bases, nucleotides of RNA bases, inosine, nucleotides of inosine, and homologues, analogues and derivatives thereof.

2. The composition of claim 1, wherein the oxidized glutathione comprises amino acids in the L-form.

3. The composition of claim 1, wherein the oxidized glutathione comprises two chemically equivalent aminoacids in the D-form and the remaining amino acid in the L-form.

4. The composition of claim 1, wherein the nitrogenous base comprises a purine.

5. The composition of claim 1, wherein the nitrogenous base comprises pyrimidine.

6. The composition of claim 1, wherein the nitrogenous base comprises inosine.

7. The composition of claim 6, wherein the oxidized glutathione and inosine are present in the composition in a molar ratio of about 1:1.

8. The composition of claim 1, wherein the homologues comprise a compound selected from 5-methyl-cytosine and dihydrouracil.

9. The composition of claim 1, wherein the analogue comprises 4-thiouracil.

10. The composition of claim 1, wherein the nucleotide comprises a phosphate selected from monophosphate, diphosphate and triphosphate.

11. The composition of claim 1, wherein the oxidized glutathione is an anion and the nitrogenous base is a cation.

12. The composition of claim 11, wherein the oxidized glutathione comprises a glycine residue which exists as a carboxylate, and the nitrogenous base includes a protonated nitrogen atom.

13. The composition of claim 1, wherein the at least one counterion is a first type of counterion, the composition further comprising at least one second type of counterion comprising an inorganic counterion selected from ammonium cations, alkaline metals, alkaline earth metals and transition metals.

14. The composition of claim 1, wherein the composition comprises at least two counterions, each counterion comprising a nitrogenous base.

15. The composition of claim 14, wherein each nitrogenous base comprises a different structure.

16. The composition of claim 15, wherein at least one nitrogenous base is a purine and least one nitrogenous base is a pyrimidine.

17. The composition of claim 15, wherein at least one nitrogenous base is a nucleoside of purine and at least one nitrogenous base is a nucleoside of pyrimidine.

18. The composition of claim 15, wherein at least one nitrogenous base is a nucleotide of purine and at least one nitrogenous base is a nucleotide of pyrimidine.

19. A composition comprising an oxidized glutathione covalently bonded to inosine.

20. The composition of claim 19, wherein the oxidized glutathione is covalently bonded to inosine via a phosphoramide linkage.

21. The composition of claim 19, wherein a ratio of the oxidized glutathione to inosine is 1:1.

22. The composition of claim 19, wherein the inosine is provided as a nucleoside or nucleotide.

23. A method of making an organic salt, comprising:
providing a solution comprising glutathione in anionic form;
oxidizing the glutathione; and
adding at least one nitrogenous base to the solution, the at least one nitrogenous base being selected from DNA bases, nucleosides of DNA bases, nucleotides of DNA bases, RNA bases, nucleosides of RNA bases, nucleotides of RNA bases, inosine, nucleotides of inosine, and homologues, analogues and derivatives thereof.

24. The method of claim 23, wherein the at least one nitrogenous base comprises inosine.

25. The method of claim 24, wherein a molar ratio of inosine to oxidized glutathione is about 1:1.

26. The method of claim 23, wherein the step of providing the solution comprising glutathione in anionic form comprises providing glutathione in a basic solution.

27. The method of claim 23, wherein the step of oxidizing the glutathione comprises adding an oxidant to the solution.

28. The method of claim 27, wherein the oxidant comprises at least one compound selected from hydrogen peroxide, air, oxygen, and a catalyst including iodine, nitrogen oxides and sulfur compounds having an active oxygen.

29. A method of making an organic salt, comprising:
providing a solution comprising oxidized glutathione; and
adding at least one nitrogenous base to the solution, the at least one nitrogenous base being selected from DNA bases, nucleosides of DNA bases, nucleotides of DNA bases, RNA bases, nucleosides of RNA bases, nucleotides of RNA bases, inosine, nucleotides of inosine, and homologues, analogues and derivatives thereof.

30. The method of claim 29, wherein the at least one nitrogenous base is inosine.

31. The method of claim 30, wherein a molar ratio of oxidized glutathione to inosine is about 1:1.

32. The method of claim 29, wherein the step of providing the solution comprises providing the oxidized glutathione in a basic solution.

33. A method of making an oxidized glutathione derivative, comprising:
reacting a glutamic acid or glutamate of oxidized glutathione or oxidized glutathione salt with phosphoric acid or a phosphoric acid derivative.

34. The method of claim 33, wherein the phosphoric acid or phosphoric acid derivative is further bonded to a nucleoside selected from the group consisting of a purine nucleoside, a pyrimidine nucleoside and an inosine nucleoside.

35. The method of claim 33, wherein the phosphoric acid derivative is further bonded to an inosine nucleotide.

36. A method of treating an infectious disease, comprising:
introducing into a subject an effective amount of a composition to achieve a therapeutic effect, the composition comprising an oxidized glutathione salt and at least one counterion of the oxidized glutathione comprising a nitrogenous base.

37. The method of claim 36, wherein the disease is a bacterial infectious disease.

38. The method of claim 36, wherein the nitrogenous base comprises a compound selected from DNA bases, nucleosides of DNA bases, nucleotides of DNA bases, RNA bases, nucleosides of RNA bases, nucleotides of RNA bases, inosine, nucleotides of inosine, and homologues, analogues and derivatives thereof.

39. The method of claim 36, wherein the subject has a disease selected from the group consisting of viral infectious diseases, bacterial infectious diseases, anaerobic infections, chlamydia infections, mycoplasma infections, mycoses and protozoa infections.

40. The method of claim 36, wherein the subject has a condition selected from virus-infected cells, macrophages containing tuberculosis mycobacteria, cells infected with mycoplasma, chlamydia and malaria plasmodium, and
the composition comprises at least one compound selected from GSSG·inosine, GSSG·uracil, GSSG·thymine, GSSG·adenosine, GSSG·guanine, GSSG·inosine-monophosphate, GSSG-uracil-monophosphate, GSSG-thymidine-monophosphate and GSSG-cytosine-monophosphate.

41. The method of claim 36, wherein the subject is infected with hepatitis C virus, and
the composition comprises at least one compound selected from GSSG·inosine, GSSG-inosine-monophosphate and GSSG-uracil-monophosphate.

42. The method of claim 36, wherein the composition is GSSG·inosine.

43. The method of claim 36, wherein the subject has a disease selected from the group consisting of hepatitis B, hepatitis C, and a combination thereof, and
the composition is GSSG·inosine.

44. The method of claim 36, wherein the subject has a disease selected from the group consisting of hepatitis B, hepatitis C, and a combination thereof, and
the composition is GSSG-inosine-monophosphate.

45. The method of claim 36, wherein the subject has acute viral hepatitis B, and
the composition comprises at least one compound selected from GSSG·inosine and GSSG-inosine-monophosphate.

46. The method of claim 36, wherein the subject has a disease selected from the group consisting of chronic hepatitis B, chronic hepatitis C, toxic hepatitis, post-alcoholic liver disease, liver cirrhosis, hepatocellular carcinoma and combinations thereof, and
the composition is GSSG·inosine.

47. The method of claim 36, wherein the subject has acute viral hepatitis C, and
the composition comprises at least one compound selected from GSSG·inosine, GSSG·uracil and GSSG-inosine-monophosphate.

48. The method of claim 36, wherein the subject has chronic viral hepatitis B, and
the composition comprises at least one compound selected from GSSG·inosine, GSSG·adenosine, GSSG·guanosine, GSSG-inosine-monophosphate and GSSG-thymidine-monophosphate.

49. The method of claim 36, wherein the subject has chronic viral hepatitis C, and
the composition comprises at least one compound selected from GSSG·inosine, GSSG·uracil, GSSG·cytosine, GSSG·dihydrouracil, GSSG-uracil-monophosphate, GSSG-cytosine-monophosphate and uracil-GSSG-inosine.

50. The method of claim 36, wherein the subjet has chronic viral hepatitis in cirrhotic stage, and
the composition comprises at least one compound selected from GSSG·inosine, GSSG·uridine, GSSG·thymidine, Li₂-GSSG-inosine-monophosphate and Na₂-GSSG-thymidine-monophosphate.

51. The method of claim 36, wherein the subject has lung tuberculosis, and
the composition comprises at least one compound selected from GSSG·inosine, GSSG·cytosine, GSSG-5-methylcytosine and Li₂-GSSG-inosine-monophosphate.

52. The method of claim 36, wherein the subject has urogenital tuberculosis, and
the composition comprises at least one compound selected from GSSG·thymine, Na₂-GSSG-guanosine-monophosphate and uracil-Li₂-GSSG-guanosine-monophosphate.

53. The method of claim 36, wherein the subject has a disease selected from the group consisting of AIDS, cytomegalovirus infection, infection caused by Epstein-Barr virus and infection caused by pneumocysts, and
the composition comprises at least one compound selected from GSSG·inosine, GSSG·dihydrouracil, GSSG-4-thiouracil, Zn₂-GSSG-thymidine-monophosphate, Ag₂-GSSG-uracil-monophosphate and uridine∞GSSG·inosine.

54. The method of claim 36, wherein the subject has herpetic infection, and
the composition comprises at least one compound selected from GSSG·inosine, Li₂-GSSG-guanosine-monophosphate, the D-form of Na₂-GSSG-cytosine-monophosphate and the D-form of GSSG·uracil.

55. The method of claim 36, wherein the subject has candidiasis, and
the composition comprises at least one compound selected from GSSG·uridine, GSSG-4-thiouracil and Ag₂-GSSG-uracil-monophosphate.

56. The method of claim 36, wherein the subject has mycoplasma infection, and
the composition comprises at least one compound selected from GSSG·inosine, GSSG·adenosine and Na₂-GSSG-adenosine-monophosphate.

57. The method of claim 36, wherein the subject has chlamydia infection, and
the composition comprises at least one compound selected from GSSG·inosine, GSSG·thymine, GSSG·uridine, GSSG·guanosine and Na₂-GSSG-guanosine-monophosphate.

58. The method of claim 36, wherein the subject has a disease selected from the group consisting of malaria and leishmaniasis, and
the composition comprises at least one compound selected from GSSG·inosine, GSSG·cytosine and GSSG-5-methylcytosine.

59. The method of claim 36, wherein the subject has an anaerobic infection, and
the composition comprises at least one compound selected from the D-form of GSSG·inosine (D-cysteine) and the D-form of GSSG·uracil (D-glutamic acid).

60. The method of claim 36, wherein the subject has a disease selected from the group consisting of viral hepatitis A, dysentery and cholera, and
the composition comprises at least one compound selected from GSSG·inosine, GSSG-inosine-monophosphate and the D-form of GSSG·uracil (D-glutamic acid).

61. The method of claim 36, wherein the subject has infectious meningitis, and
the composition comprises at least one compound selected from GSSG·inosine, Li₂-GSSG-inosine-monophosphate, the D-form of GSSG·uracil (D-glutamic acid), GSSG-5-methylcytosine and Ag₂-GSSG-uracil-monophosphate.

62. The method of claim 36, wherein the subject has a disease selected from the group consisting of plague, tularemia and anthrax, and
the composition comprises at least one compound selected from GSSG·inosine, GSSG·adenine, GSSG·thymine, GSSG-5-methylcytosine, GSSG-4-thiouracil, the D-form of GSSG·uracil (D-glutamic acid), the D-form of GSSG·inosine (D-cysteine) and adenine·GSSG·thymine.

63. The method of claim 36, wherein the subject has an infection caused by prions, and
the composition comprises at least one compound selected from GSSG·inosine, GSSG·uridine, GSSG·dihydrouracil, Ag₂-GSSG-uracil-monophosphate, Ag₂-GSSG-thymidine-monophosphate and uracil-monophosphate-Li₂-GSSG-inosine-monophosphate.

64. The method of claim 36, wherein the subject has a disease selected from the group consisting of flu and acute respiratory infections, and
the composition comprises at least one compound selected from GSSG·inosine, GSSG·adenosine, GSSG·uracil and GSSG·thymine.

65. The method of claim 36, wherein the composition is introduced into the subject via a pharmaceutically acceptable solvents or vehicles through parenteral and oral routes, inhalation solutions, solutions for local instillations, eye or intranasal drops, ointments, creams or gels, and suppositories.

66. A method for treating a subject in need treatment of a condition caused by replicative activity of viruses, bacteria or other infecting agents, comprising:
introducing into the subject in need of such treatment a composition to obtain a therapeutic effect, the composition comprising an oxidized glutathione salt, and at least one counterion of the oxidized glutathione comprising a nitrogenous base.

67. A method for treating a subject, comprising:
introducing into the subject in need of such treatment a composition to obtain a cell-protective effect, the composition comprising an oxidized glutathione salt, and at least one counterion of the oxidized glutathione comprising a nitrogenous base.
